SEQUENCE LISTING (10 sequences)

SEQ ID NO: 1 - DNA, 835 bp, Homo sapiens
  human caspase-3 gene
aatggagaacactgaaaactcagtggattcaaaatccattaaaaatttggaaccaaagatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcactaa

SEQ ID NO: 2 - protein, 277 aa, Homo sapiens
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 3 - DNA, 1910 bp, Homo sapiens
  human MST1 gene
ccggctgctggcatcggccatggagacggtacagctgaggaacccgccgcgccggcagctgaaaaagttggatgaagatagtttaaccaaacaaccagaagaagtatttgatgtcttagagaaacttggagaagggtcctatggcagcgtatacaaagctattcataaagagaccggccagattgttgctattaagcaagttcctgtggaatcagacctccaggagataatcaaagaaatctctataatgcagcaatgtgacagccctcatgtagtcaaatattatggcagttattttaagaacacagacttatggatcgttatggagtactgtggggctggttctgtatctgatatcattcgattacgaaataaaacgttaacagaagatgaaatagctacaatattacaatcaactcttaagggacttgaataccttcattttatgagaaaaatacaccgagatatcaaggcaggaaatattttgctaaatacagaaggacatgcaaaacttgcagattttggggtagcaggtcaacttacagataccatggccaagcggaatacagtgataggaacaccattttggatggctccagaagtgattcaggaaattggatacaactgtgtagcagacatctggtccctgggaataactgccatagaaatggctgaaggaaagcgcccttatgctgatatccatccaatgagggcaatcttcatgattcctacaaatcctcctcccacattccgaaaaccagagctatggtcagataactttacagattttgtgaaacagtgtcttgtaaagagccctgagcagagggccacagccactcagctcctgcagcacccatttgtcaggagtgccaaaggagtgtcaatactgcgagacttaattaatgaagccatggatgtgaaactgaaacgccaggaatcccagcagcgggaaatggaccaggacgatgaagaaaactcagaagaggatgaaatggattctggcacgatggttcgagcagtgggtgatgagatgggcactgtccgagtagccagcaccatgactgatggagccaatactatgattgagcacgatgacacgttgccatcacaactgggcaccatggtgatcaatgcagaggatgaggaagaggaaggaactatgaaaagaagggatgagaccatgcagcctgcgaaaccatcctttcttgaatattttgaacaaaaagaaaaggaaaaccagatcaacagctttggcaagagtgtacctggtccactgaaaaattcttcagattggaaaataccacaggatggagactacgagtttcttaagagttggacagtggaggaccttcagaagaggctcttggccctggaccccatgatggagcaggagattgaagagatccggcagaagtaccagtccaagcggcagcccatcctggatgccatagaggctaagaagagacggcaacaaaacttctgagcaaggccaggctgtgagggccccagctccacccaggctttgggtgaattctggatggcttgctcatgtttgttagccagcaccttctgctctgtcgtctctccacagcacctttgtgaactcaggaatgtgcgccagtgggaagggctctcttgacagtcagcgtgccatcttgatgtgtgtatgtacattggtcaggtatattatctcaaaggatttatattgggcgacttttaactcagagttttaaaccccaggaacagagactcctagttgagtgatagctgggaaagttttacattgtctgtttttcttctcccaatagctttcaattgttctttctggaagacttttaaaaaaatataaatatgcatatatatatataaattataaatagattccccacgcaggttggtggcatctctgt

SEQ ID NO: 4 - protein, 487 aa, Homo sapiens [sequence omitted]

SEQ ID NO: 5 - DNA, 30 bp, Artificial Sequence
  primer
atgaccatgattacgaattcatggagacac

SEQ ID NO: 6 - DNA, 30 bp, Artificial Sequence
  primer
cactctagattaattaaaaaaatagagttc

SEQ ID NO: 7 - protein, 268 aa, Homo sapiens
SKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 8 - protein, 249 aa, Homo sapiens
SGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 9 - protein, 147 aa, Homo sapiens
SGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETD

SEQ ID NO: 10 - protein, 102 aa, Homo sapiens
SGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

(19) United States
(12) Patent Application Publication
(10) Pub. No.: US 2006/0013804 A1
(43) Pub. Date: Jan. 19, 2006

(54) Modulation of stem cell differentiation by modulation of caspase-3 activity

(75) Inventors: Lynn Megeney, Ottawa (CA); Pasan Fernando, Ottawa (CA)

Correspondence Address: KING & SPALDING LLP, 191 PEACHTREE STREET, N.E., 45TH FLOOR, ATLANTA, GA, 30303-1763, US

(73) Assignee: Ottawa Health Research Institute, Ottawa (CA)

(21) Appl. No.: 11/187,410

(22) Filed: Jul. 22, 2005

Related U.S. Application Data
(63) Continuation-in-part of application No. 10/538,767.
(60) Provisional application No. 60/431,991, filed on Dec. 10, 2002.
(60) Provisional application No. 60/431,990, filed on Dec. 10, 2002.

(51) Int. Cl. A61K 45/00; C12N 5/08

(52) U.S. Cl. 424/93.7; 435/368

(57) ABSTRACT

Methods of directing stem cell fate for therapeutic purposes through the deliberate manipulation of caspase-3 activity are provided. The use of modulators of caspase-3 activity to modulate stem cell differentiation is described including activators and/or effectors of caspase-3, which can be used to induce stem cell differentiation, and inhibitors of caspase-3, which can be used to inhibit differentiation and thereby promote or maintain proliferation of stem cells. Methods of screening for modulators of caspase-3 and the use of such compounds to modulate stem cell differentiation in vitro or in vivo are also provided as are therapeutic applications of the compounds.

### FIELD OF INVENTION

The present invention pertains to the field of stem cell therapeutics and in particular to methods of modulating stem cell differentiation through the activation or inhibition of caspase-3 protein.

### BACKGROUND OF THE INVENTION

Stem cells are undifferentiated, or immature, cells that are capable of giving rise to multiple, specialised cell types and ultimately to terminally differentiated cells. Unlike any other cells, they are able to renew themselves such that essentially an endless supply of mature cell types can be generated when needed. Due to this capacity for self-renewal, stem cells are therapeutically useful for the regeneration and repair of tissues.

In vertebrate organisms, development of tissue and organ systems is mediated by expansion of previously committed stem cell lineages, followed by terminal differentiation. The therapeutic utility of stem cells (i.e. the ability to regenerate damaged or defective tissue) is dependent upon obtaining a sufficient number of target cells and being able to manipulate the transition to differentiated mature tissue specific cells. Recent investigations, therefore, have centred on exploring methods to culture stem cells in vitro in order to increase the numbers of these cells. Over the last few years some progress has been made in understanding stem cell differentiation, discovery of cytokines, isolation and identification of cellular subtypes and in the development of a variety of bioreactor concepts (see, for example, the review by Noll et al., (2002) Adv Biochem Eng Biotechnol 74:111-28) and studies attempting to expand stem or progenitor cells in vitro or ex vivo have become possible due to the availability of recombinant growth factors and cell selection technologies. However, the controlled expansion and differentiation of stem cells remains one of the most challenging fields in cell culture.

Although methods of stimulating stem cell proliferation have been described, they are mainly limited to specific stem cell types, for example, haematopoietic stem cells (see, U.S. Pat. Nos. 5,981,708 and 5,728,581) or neural stem cells (see U.S. Pat. No. 5,750,376). Furthermore, the transition to differentiation is not well understood, but is known to be dependent on a co-ordinated response involving inhibition of growth promoting gene products and up-regulation of tissue-specific transcription factors.

Skeletal myogenesis has served as an ideal model system in which to explore the basic precepts of regulatory control governing cellular differentiation. Recent evidence has demonstrated that select signal transduction pathways may provide the impetus to initiate muscle differentiation and a promyogenic role has been assigned to the mitogen-activated protein kinase (MAPK) pathways. For example, activation of p38α, a member of the p38 subfamily of MAPKs, is concurrent with the induction of differentiation, and use of pharmacologic inhibitors of p38α can effectively block this process (Cuenda, A., & Cohen, P., (1999) J. Biol. Chem. 274, 4341-4346; Wu, Z., et al., (2000) Mol. Cell. Biol. 20, 3951-3964; Zetser, A., et al., (1999) J. Biol. Chem. 274, 5193-5200; Ornatsky, O. I., et al., (1999) Nucleic Acids Res. 27, 2646-2654). A promyogenic effect has also been attributed to p38γ, although the mechanism by which this kinase facilitates myogenesis remains to be identified (Graves, J. D., et al., (1998) EMBO J 17, 2224-2234).

The p38 MAPK family has also been implicated in the initiation and progression of apoptosis (Juo, P., et al., (1997) Mol. Cell. Biol. 17, 24-35; Hall, A., & Nobes, C. D. (2000) Philos Trans R Soc Lond B Biol Sci 355, 965-970). A number of observations have indicated that apoptosis and differentiation may utilise the same signal cascade to engage MAPK activity in muscle cells. For example, actin fibre disassembly/reorganisation is a conserved feature of both apoptosis (Sabourin, L. A., & Rudnicki, M. A. (2000) Clin. Genet. 57, 16-25; Gallo, R., et al., (1999) Mol. Biol. Cell 10, 3137-3150) and differentiating myoblasts (Qu, G., et al., (1997) J. Cell. Biochem. 67, 514-527; Mills, J. C., et al., (1998) J. Cell Biol. 140, 627-636). Secondly, the conserved muscle contractile protein, myosin light chain kinase, is required for the apoptotic feature of membrane blebbing (Powell, W. C., et al., (1999) Curr. Biol. 9, 1441-1447). Finally, increased activity of matrix metalloproteinases appears to be an indispensable requirement for orchestrating membrane fusion in both myoblast differentiation and apoptosis (Yagami-Hiromasa, T., et al., (1995) Nature 377, 652-656; Utz, P. J., & Anderson, P. (2000) Cell Death Differ. 7, 589-602).

Successful completion of the apoptotic program is dependent on the activity of a unique class of proteolytic enzymes referred to as caspases (Utz, P. J., & Anderson, P. (2000) Cell Death Differ. 7, 589-602). Although caspase proteins primarily target and inactivate proteins through serine directed cleavage events, various caspase proteins also engage apoptosis through cleavage activation of signalling molecules including MEKK1 (Cardone, M. H., et al., (1997) Cell 90, 315-323) and SLK (Hall, A., & Nobes, C. D. (2000) Philos Trans R Soc Lond B Biol Sci 355, 965-970). In addition, caspase-3 activity has been linked with activation of the MAPKs JNK and p38, albeit through activation of intervening kinases (Utz, P. J., & Anderson, P. (2000) Cell Death Differ. 7, 589-602; Cardone, M. H., et al., (1997) Cell 90, 315-323; Chaudhary, P. M., et al., (1999) J. Biol. Chem. 274, 19211-19219). A recent report has also indicated that early initiation of the skeletal myogenic program relies on the activity of caspase-3, a key apoptotic serine protease (Fernando et al. (2002) PNAS 99:11025-30). This report indicated that primary myoblasts transfected with recombinant active caspase-3 underwent differentiation and that the caspase 3-activated kinase, Mammalian Sterile Twenty-like kinase (MST1), was able to rescue myogenesis in caspase 3^{-/-} myoblasts.

Although methods of stimulating proliferation of stem cells have been described and advances in the understanding of differentiation induction in stem cells have been made, these are limited to specific, usually pre-committed, stem cell types. A need remains, therefore, for a method of modulating stem cell differentiation that is more widely applicable.

### SUMMARY OF THE INVENTION

An object of the present invention is to provide methods of modulating stem cell differentiation using modulators of caspase-3 activity. In accordance with one aspect of the present invention, there is provided a method of screening for compounds that modulate stem cell differentiation comprising:
(a) identifying a compound that modulates the activity of caspase-3 by contacting a caspase-3 protein with a candidate compound, measuring the activity of the caspase-3 protein and comparing the measured activity with the activity of caspase-3 protein in the absence of the candidate compound, wherein a difference in the activities indicates that the candidate compound is a modulator of caspase-3 activity;
(b) contacting a population of stem cells with said modulator of caspase-3 activity to provide a treated population of stem cells;
(c) measuring the level of at least one marker of differentiation in said population of stem cells, and
(d) comparing the level of said marker in the treated population of stem cells with a control population of stem cells that have not been contacted with said modulator,
wherein a difference in the levels of said marker indicates that the modulator is a compound capable of modulating stem cell differentiation.

In accordance with another aspect of the present invention, there is provided a use of a caspase-3 protein, or a polynucleotide encoding a caspase-3 protein, to screen for compounds that modulate stem cell differentiation.

In accordance with another aspect of the present invention, there is provided a use of one or more compound that modulates caspase-3 activity to modulate differentiation of stem cells.

In accordance with another aspect of the present invention, there is provided a method of modulating stem cell differentiation comprising contacting a stem cell, or a population of stem cells, with one or more modulators of caspase-3 activity.

In accordance with another aspect of the present invention, there is provided a method as described above, wherein the stem cell, or population of stem cells, are contacted sequentially with a modulator that attenuates the activity of caspase-3 and inhibits stem cell differentiation and a modulator that increases the activity of caspase-3 and induces stem cell differentiation.

In accordance with another aspect of the present invention, there is provided a method for producing a pharmaceutical composition for modulating differentiation of stem cells comprising: identifying a compound by the screening method of the invention and formulating said compound into a pharmaceutically acceptable form.

### BRIEF DESCRIPTION OF THE FIGURES

FIG. 1 depicts deficient myotube formation in caspase-3 null myoblasts;

FIG. 2 demonstrates that caspase-3 activity is required for skeletal muscle differentiation;

FIG. 3 depicts induction of differentiation in growing myoblasts by activated caspase-3;

FIG. 4 depicts the activation of Mammalian Sterile Twenty-like kinase (MST1) by caspase-3 during skeletal muscle differentiation;

FIG. 5 depicts the rescue of the caspase-3_{-/-} myoblast phenotype by activated MST1;

FIG. 6 depicts the nucleotide sequence for the human caspase-3 gene [Genbank Accession No. gi|857568; SEQ ID NO:1];

FIG. 7 depicts (A) the amino-acid sequence for the human caspase-3 protein [Genbank Accession No. gi|857569; SEQ ID NO:2], (B) the amino-acid sequence for one form of active caspase-3 protein (cleavage between amino acids 9 and 10) [SEQ ID NO:7], (C) the amino-acid sequence for another form of active caspase-3 protein (cleavage between amino acids 28 and 29) [SEQ ID NO:8], (D) and (E) the amino-acid sequences for two possible sub-units of active caspase-3 protein (cleavage of SEQ ID NO:8 between amino acids 175 and 176)[SEQ ID NO:9 (D) and SEQ ID NO:10 (E)];

FIG. 8 depicts the nucleotide sequence for the human MST1 gene [Genbank Accession No. gi|1117790; SEQ ID NO:3];

FIG. 9 depicts the amino-acid sequence for the human MST1 protein [Genbank Accession No. gi|1117791; SEQ ID NO:4];

FIG. 10 depicts the distribution of pro-caspase 3 and active caspase 3 in myoblasts grown under (A) growth conditions, (B) differentiation conditions for 12 h, (C) differentiation conditions for 24 h, and (D) differentiation conditions for 48 h;

FIG. 11 depicts the distribution of (A) pro-caspase 3 and (B) active caspase 3 in primary striatal stem cells cultured under growth conditions and differentiation conditions (for 12 h, 24 h, and 48 h);

FIG. 12 depicts striatal stem cells during proliferation in the presence or absence of a caspase-3 inhibitor (A) stained for nestin, (B) stained for myelin basic protein and (C) stained for glial fibrillary acid protein; and

FIG. 13 depicts striatal stem cells after 48 h differentiation in the presence or absence of a caspase-3 inhibitor (A) stained for nestin, (B) stained for myelin basic protein and (C) stained for glial fibrillary acid protein.

FIG. 14 shows Caspase 3 activity is detectable in differentiating neurospheres. (A) Fluorometric detection of caspase 3 activity during normal differentiation of CD1 neurospheres. (B) Immunocytochemical staining of active caspase 3 in clonally-derived CD1 neurospheres after incubation in differentiation media. Active caspase 3 was visualized by counterstaining with a anti-FITC conjugated secondary antibody. Nuclei were visualized with DAPI. Solid arrows demonstrate the localization of active caspase 3 and open arrows illustrate condensed cell bodies. (C) Immunoblot analysis of active caspase 3 from CD1 neurospheres showing an increase in active caspase 3 (17 kDa caspase 3 fragment) after 24 h of differentiation. (D) Immunoblot analysis of PARP during neurosphere differentiation. The 118 kDa full length protein is shown (top arrow). The 85 kDa cleaved fragment was not present in differentiating neurospheres (bottom arrow) indicating that the elevated caspase 3 activity did not lead to apoptosis. Immunofluorescence staining in panel b included an unstained and secondary (FITC-conjugated) antibody as controls (not shown).

FIG. 15 shows results of culturing neurosphere cells in differentiation media under control (DMSO) or caspase 3 inhibited (DEVD) conditions for 12 or 24 h. (A) Annexin-V-PI analysis of neurosphere cells after 12 h of culture in differentiation media. (b) Annexin-V-PI analysis of neurosphere cells after 24 h of culture in differentiation media. R1 represents the total heterogeneity/granularity index of the cells as a strategy to exclude cellular debris. The results are representative of 4 independent trials.

FIG. 16 shows results indicating Caspase 3 activity promotes neurosphere differentiation. Clonally-derived neurospheres were incubated with (DEVD) or without (DMSO) a caspase 3 peptide inhibitor (z-DEVD.fmk). (A) Both DEVD and DMSO treated neurospheres maintained under growth conditions express nestin (Cy3-red). (B) DEVD treated neurospheres incubated in differentiation media for 48 h remain in an early progenitor state and express nestin whereas control treated neurospheres lose nestin expression. (C) DEVD treated neurospheres fail to express the astrocyte marker protein GFAP (FITC-green). (D) DEVD treated neurospheres fail to express the oligodendrocyte marker protein MBP (FITC-green). In all panels, cells were counterstained with DAPI (blue) to visualize nuclei. Cells were photographed at 60× magnification. Cells were stained with secondary antibody to control, for non-specific background (not shown).

FIG. 17 shows results indicating reduced accumulation of neuro-specific proteins in caspase 3 inhibited and caspase 3 null neurospheres during differentiation. Clonally derived CD1 neurospheres were subject to immunoblot analyses after cell culture treatment. (A) Accumulation of the cleaved (active) 21 kDa caspase 3 fragment. The corresponding graph represents densiometric quantitation of active caspase 3 immunoblots from control treated cells (n=4). Values are expressed relative to a N1E-115 neuroblastoma control treated with actinomycin D. Control cells demonstrated changes in active caspase 3 that were similar to those observed by immunofluoresence (see FIG. 14). (B) PARP was not cleaved during normal neurosphere differentiation suggesting that these neurospheres were not undergoing apoptosis. (C) The accumulation of neuronal class III β-tubulin (Tuj-1) was lower in caspase 3 inhibited samples relative to control (DMSO) neurospheres. Immunoblots were quantified to compare relative levels of Tuj-1. Values were expressed relative to a sample from differentiated neuroblastoma cells (N1E-115) (n=3). (d) The expression of nestin remained elevated in DEVD treated neurospheres indicating that these neurosphere retain their early progenitor phenotype. The level of nestin within each condition was expressed relative to the amount of nestin at growth (G) (n=4). In panels a-d, differentiation was assessed at growth (G) and at the indicated time point (days) thereafter. p38α is shown as a loading control. (e) Immunoblot analysis of pro-caspase 3 from E14.5 day caspase 3 wildtype (+/+), heterozygous (+/−) and null (−/−) embryos. p38α is shown as a loading control. (f) Neurospheres generated from caspase 3 mutant mice were analyzed during growth (G) or after a 48 h incubation in differentiation media. (D). Nestin remained elevated in caspase 3 null neurospheres under growth and differentiation conditions. The oligodendrocyte marker 2′3′ cyclic nucleotide 3′ phosphohydrolase (CNPase) accumulated in neurospheres from both caspase 3 wildtype and heterozygous embryos following incubation in differentiation media. CNPase was not detected in neurospheres from caspase 3 null embryos. p38α is shown as a loading control.

FIG. 18 shows results indicating altered cellular morphology in caspase 3 inhibited neurospheres. Clonally-derived CD1 neurospheres were treated with either a caspase 3 inhibitor (DEVD), a caspase 8 inhibitor (IETD) or kept as a control (DMSO) and then incubated with differentiation media for the indicated periods (days). (A) Cells were visualized under transmission light microscopy. Images at 24 h were taken with a 10× objective, all other images were taken at 20× to visualize neurite outgrowth. A representative enlargement demonstrating neurite outgrowth with each treatment is shown at day 4 (inset). The extensive neurite networking present in control and caspase 8 inhibited samples was absent in caspase 3 inhibited cells. (B) Quantitation of neurite outgrowth in neurospheres after 2 and 4 days of differentiation in DMSO, DEVD, or IETD. The photomicrographs from panel (A) are representative of 3 independent trials.

FIG. 19 shows results indicating that inhibition of caspase 3 activity leads to a reduction in pro-neurogenic kinases. Neurospheres were incubated with or without a caspase 3 peptide inhibitor and maintained in differentiation media for up to five days. The autoradiographs demonstrate the phosphorylation of myelin basic protein after immunoprecipitation/kinase analysis and Western transfer of (A) PAK1, (C) ASK1 and (E) p38α. Immuno-assays of ASK1, PAK1 and p38α (lower panels of a, c and e respectively) were performed to verify equal amounts of protein following immunoprecipitation. Liquid scintillation analysis was used to determine the relative amount of 32P incorporation for (B) PAK1, (D) ASK1 and (F) p38α. A recombinant active MAPKAP kinase-2 protein was used as an internal control for each trial. The graphs are representative of 3-4 independent trials per kinase.

### DETAILED DESCRIPTION OF THE INVENTION

The present invention is based on the elucidation of an unanticipated role of a caspase-3 mediated signal cascade in the modulation of stem cell differentiation. The invention thus provides for a means of directing stem cell fate for therapeutic purposes through the deliberate manipulation of caspase-3 activity.

Accordingly, the present invention provides for the use of modulators of caspase-3 activity to modulate stem cell differentiation, wherein the modulators exert their effect by inhibiting or activating one or more component of the caspase-3 signalling pathway. The modulators of the present invention can be activators and/or effectors of caspase-3, which can be used to induce stem cell differentiation, or inhibitors of caspase-3, which can be used to inhibit differentiation and thereby promote or maintain proliferation of stem cells.

Thus, in one embodiment of the invention, there are provided methods of inducing stem cell differentiation by inducing the caspase-3 signalling pathway and activators and effectors of the caspase-3 protein for use in the induction of stem cell differentiation.

In another embodiment of the present invention, there are provided methods of inhibiting stem cell differentiation, and thereby promoting proliferation, and inhibitors of the caspase-3 protein for use in promoting stem cell proliferation.

The present invention further contemplates stepwise manipulation of stem cell fate, for example, by sequentially contacting a stem cell population first with one or more caspase-3 inhibitor in order to promote expansion of the stem cells, and subsequently with one or more activator/effector of caspase-3 in order to induce differentiation of the expanded stem cell population.

The methods and caspase-3 modulators (activators, effectors and inhibitors) described herein can be used, for example, to stimulate stem cell differentiation in vitro in order to provide replacement tissue, to stimulate stem cell differentiation in vivo in order to replace or repair damaged tissue, to promote stem cell proliferation in vivo in order to aid in the replacement or repair of damaged tissue in a subject in situ, or to promote the ex vivo proliferation of stem cells and thereby provide a population of cells suitable for transplantation. Therapeutic applications of the invention pertain to diseases and disorders in which there is a need to promote stem cell differentiation and/or replace damaged or defective tissue such as muscular dystrophy, cardiovascular disease, stroke, heart failure, myocardial infarction, neurodegenerative diseases, and the like, as well as diseases and disorders in which there is a need to replace damaged or malfunctioning tissue, such as degenerative liver diseases, including cirrhosis and hepatitis, diabetes, neurodegenerative disorders, such as Parkinson's disease and Alzheimer's disease, and degenerative or ischemic cardiac disease.

Definitions

Unless defined otherwise, all technical and scientific terms used herein have the same meaning as commonly understood by one of ordinary skill in the art to which this invention pertains.

As employed throughout the disclosure, the following terms, unless otherwise indicated, shall be understood to have the following meanings:

The term “caspase-3,” as used herein, refers to mammalian caspase-3 protein and biologically active fragments thereof. Caspase-3 is a serine protease which is capable of cleaving other polypeptides and/or proteins and is also known as CPP32, Yama and apopain. Caspase-3 cleaves most efficiently at the cleavage motif DEVD but is also capable of cleaving at other sites (including those with a DXXD motif), albeit with lower efficiency. As is known in the art, caspase-3 is synthesized as a proenzyme comprising an N-terminal peptide (PRO-domain) and two sub-units (see review by Cohen, G. M., Biochem. J., (1997) 326:1-16). The PRO-domain is cleaved off the proenzyme to provide active caspase-3. Both the pro-form and various active form(s) of the protein are, therefore, encompassed by the term “caspase-3.” In the human enzyme, the proposed cleavage site for the PRO-domain is either Asp-9 or Asp-28, or both. Formation of the two sub-units of active caspase-3 is proposed to take place by an additional cleavage at amino acid residue Asp-175. Further processing may take place in one or both of the subunits. Thus, the active form of caspase-3 may comprise a single polypeptide, two or more sub-unit polypeptides, or a mixture of these components. For example, many caspases are active as a heterotetramer comprising two of each subunit.

The term “caspase-3 signalling pathway,” as used herein, refers to the cellular signalling pathway that is mediated by the caspase-3 protein and which ultimately affects cell differentiation.

The term “stem cell,” as used herein, encompasses stem cells, pre-committed stem cells and progenitor cells. Thus, the term applies to totipotent, pluripotent and unipotent cells. Totipotent stem cells typically have the capacity to generate a lineage of fully differentiated, functional progeny by differentiation and proliferation. Totipotent stem cells are typically embryonic in origin, although totipotent cells have also been isolated from certain adult tissues. Unipotent and pluripotent cells are typically cells in a stem cell line capable of differentiating into one, or several different, final differentiated cell types. Unipotent and pluripotent stem cells can originate from various tissue or organ systems, including, but not limited to, blood, nerve, muscle, skin, gut, bone, kidney, liver, pancreas, thymus, and the like. In the context of the present invention, the term “stem cell” encompasses all cells in a lineage of differentiation and proliferation prior to the most differentiated or the fully mature cell. For example, a skin progenitor cell in the mature individual, which is capable of differentiation to only one type of cell, but which is itself not fully mature or fully differentiated would be included in the definition of stem cell.

The term “differentiation,” as used herein, refers to a developmental process whereby cells become specialised for a particular function, for example, where cells acquire one or more morphological characteristics and/or functions different from that of the initial cell type. Differentiation may be assessed, for example, by monitoring the presence or absence of lineage markers, using immunohistochemistry or other procedures known to a worker skilled in the art. Differentiated progeny cells derived from stem cells according to the methods of the invention may be but are not necessarily related to the same germ layer or tissue as the source tissue of the stem cells. For example, neural progenitor cells and muscle progenitor cells can differentiate into haematopoietic cell lineages.

The terms “proliferation” and “expansion,” as used interchangeably herein with reference to cells, mean an increase in the number of cells of the same type by division. In the context of the present invention, a compound is considered to “promote proliferation” if the compound increases the proliferation rate of a population of stem cells when compared to stem cells not treated with the compound, and when the compound maintains the proliferation rate of population of stem cells compared to stem cells not treated with the compound, which undergo differentiation.

As used herein, an “activator” of caspase-3 is a compound or molecule that promotes caspase-3 activity either by causing caspase-3 to become active or by increasing the activity of caspase-3 and includes caspase-3 protein itself (both pro- and active forms) as well as biologically active fragments thereof. An “effector” of caspase-3 as used herein refers to a compound or molecule that works downstream of caspase-3 in the pathway to enhance the effects of caspase-3 activity, i.e. is a part of, or activates a part of, the cascade of molecular and cellular events that occurs due to caspase-3 activation. The terms “activator” and “effector,” thus cover compounds and molecules that are capable of stimulating the caspase-3 signalling pathway in cells in which the pathway is not currently active as well as compounds or molecules that act as “enhancers” and increase the natural activity of the pathway.

The term “inhibitor” of caspase-3, as used herein, refers to a compound or molecule that attenuates the activity of caspase-3 either directly or indirectly. The term thus includes compounds/molecules that act n caspase-3 itself as well as compounds/molecules that act upstream of caspase-3 in the signalling pathway and prevent caspase-3 becoming activated. The term “inhibitor” thus covers compounds and molecules that are capable of inhibiting the caspase-3 signalling pathway in cells in which the pathway is currently active as well as compounds or molecules that prevent the activation of the pathway.

As used herein, the terms “other cell” or “second cell” or “educator cell” are used interchangeably and refer to a cell that is not the same as the stem cell in use. Typically the other cell will be a different type of stem cell, or a partially or fully differentiated non-stem cell, and may originate from the same germ layer or a different germ layer as the stem cell in use. Typically the other cell will be developmentally related to the target differentiated stem cell and will influence (educate) the developmental path of the stem cell in use. The other cells may be, for example, myoblasts, hepatocytes, pancreatic islet cells, or Sertoli cells, and may be, for example, primary or established cell lines. One skilled in the art will be able to assess the suitability of cells from various tissue sources and at various stages of differentiation to act as educator cells in the methods of the instant invention, using methods known in the art, including by exposure to stem cells in co-culture (see, for example, Seale, P., et al., (2000) Cell. 102, 777-786; Minasi, M. G., et al., (2002) Development. 129, 2773-2783; Hierlihy, A. M., et al., (2002) FEBS Lett. 530, 239-243). Exposure of stem cells to other cells may be direct exposure, for example via contact between stem cells and other cells, or indirect exposure, for example through conditioned medium, lysate or lysate factors derived from the other cells.

The term “corresponds to” as used herein with reference to nucleic acid sequences means a polynucleotide sequence that is identical to all or a portion of a reference polynucleotide sequence. In contradistinction, the term “complementary to” is used herein to mean that the polynucleotide sequence is identical to all or a portion of the complement of a reference polynucleotide sequence. For illustration, the nucleotide sequence “TATAC” corresponds to a reference sequence “TATAC” and is complementary to a reference sequence “GTATA”.

The following terms are used herein to describe the sequence relationships between two or more polynucleotides or two or more polypeptides: “reference sequence,” “window of comparison,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA, gene or protein sequence, or may comprise a complete cDNA, gene or protein sequence. Generally, a reference polynucleotide sequence is at least 20 nucleotides in length, and often at least 50 nucleotides in length. A reference polypeptide sequences is generally at least 7 amino acids in length and often at least 17 amino acids in length.

A “window of comparison”, as used herein, refers to a conceptual segment of the reference sequence of at least 15 contiguous nucleotide positions or at least 5 contiguous amino acid positions over which a candidate sequence may be compared to the reference sequence and wherein the portion of the candidate sequence in the window of comparison may comprise additions or deletions (i.e. gaps) of 20 percent or less as compared to the reference sequence (which does not comprise additions or deletions) for optimal alignment of the two sequences. The present invention contemplates various lengths for the window of comparison, up to and including the full length of either the reference or candidate sequence. Optimal alignment of sequences for aligning a comparison window may be conducted using the local homology algorithm of Smith and Waterman (Adv. Appl. Math. (1981) 2:482), the homology alignment algorithm of Needleman and Wunsch (J. Mol. Biol. (1970) 48:443), the search for similarity method of Pearson and Lipman (Proc. Natl. Acad. Sci. (U.S.A.) (1988) 85:2444), using computerised implementations of these algorithms (such as GAP, BESTFIT, FASTA, and TFASTA in the Wisconsin Genetics Software Package Release 7.0, Genetics Computer Group, 573 Science Dr., Madison, Wis.), using publicly available computer software such as ALIGN or Megalign (DNASTAR), or by inspection. The best alignment (i.e. resulting in the highest percentage of identity over the comparison window) is then selected.

The term “sequence identity” means that two polynucleotide or polypeptide sequences are identical (i.e. on a nucleotide-by-nucleotide or amino acid-by-amino acid basis) over the window of comparison.

The term “percent (%) sequence identity,” as used herein with respect to a reference sequence is defined as the percentage of nucleotide or amino acid residues in a candidate sequence that are identical with the residues in the reference polypeptide sequence over the window of comparison after optimal alignment of the sequences and introducing gaps, if necessary, to achieve the maximum percent sequence identity, without considering any conservative substitutions as part of the sequence identity.

The term “substantial identity” as used herein denotes a characteristic of a polynucleotide or polypeptide sequence, wherein the polynucleotide or polypeptide comprises a sequence that has at least 50% sequence identity as compared to a reference sequence over the window of comparison. Polynucleotide and polypeptide sequences at least 60% sequence identity, at least 70% sequence identity, at least 80% sequence identity, and at least 90% sequence identity as compared to a reference sequence over the window of comparison are also considered to have substantial identity with the reference sequence.

“Naturally occurring” as used herein, as applied to an object, refers to the fact that an object can be found in nature. For example, a naturally occurring polypeptide or polynucleotide sequence would be one that is present in an organism (including viruses), which can be isolated from the organism and which has not been intentionally modified by man in the laboratory.

As used herein, the term “about” refers to a +/−10% variation from the nominal value. It is to be understood that such a variation is always included in any given value provided herein, whether or not it is specifically referred to.

The term “subject” or “patient” as used herein refers to an animal in need of treatment.

The term “animal,” as used herein, refers to both human and non-human animals, including, but not limited to, mammals, birds and fish.

Other chemistry terms employed herein are used according to conventional usage in the art, as exemplified by The McGraw-Hill Dictionary of Chemical Terms (ed. Parker, S., 1985), McGraw-Hill, San Francisco.

Modulation of the Stem Cell Differentiation

The present invention provides for methods of directing the fate of stem cells by modulating differentiation. This is achieved by the deliberate manipulation of caspase-3 activity within the caspase-3 signalling pathway through the use of compounds that modulate caspase-3 activity. The methods of the present invention thus comprise contacting a stem cell, or a population of stem cells, with one or more modulator of caspase-3 activity. In the context of the present invention, a “modulator” can be an activator, an effector or an inhibitor.

In accordance with one embodiment of the invention, therefore, there is provided the use of activators and effectors of caspase-3 to induce stem cell differentiation through the induction or enhancement of caspase-3 activity. In accordance with another embodiment of the invention, there is provided the use of inhibitors of caspase-3 to inhibit stem cell differentiation through the attenuation of caspase-3 activity.

In the context of the present invention, a modulator of caspase-3 may be a direct modulator of caspase-3 activity which acts on the caspase-3 protein itself (or the pro-form of caspase-3), or it may be an indirect modulator which modulates the activity of one or more protein that acts upstream or downstream of caspase-3 in the signalling pathway.

1. Activators and Effectors of Caspase-3

In the context of the present invention, an activator is a compound that promotes caspase-3 activity either by causing caspase-3 to become active or by increasing the activity of caspase-3. An effector is a compound that works downstream of caspase-3 in the caspase-3 signalling pathway to enhance the effects of caspase-3 activity, i.e. is a part of, or activates a part of, the cascade of molecular and cellular events that occurs due to caspase-3 activation and which affects stem cell differentiation. Examples of such activators and effectors include, but are not limited to, the caspase-3 protein (both the pro-form and the active form), biologically active fragments of caspase-3 protein, proteins or biologically active fragments of proteins that are activated by caspase-3 in the signalling pathway, proteins or biologically active fragments of proteins that activate caspase-3 in the signalling pathway, polynucleotides encoding such proteins and fragments, expression vectors containing the polynucleotides, and other compounds that activate caspase-3, increase the activity of caspase-3 or enhance expression of caspase-3.

1.1 Proteins and Polypeptides

The proteins and polypeptides for use as activators and effectors of the present invention include the caspase-3 protein, biologically active fragments thereof, proteins or biologically active fragments of proteins that are activated by caspase-3 in the signalling pathway and proteins or biologically active fragments of proteins that activate caspase-3 in the signalling pathway. Proteins that are activated by caspase-3 in the signalling pathway include those that are directly activated through the proteolytic activity of caspase-3, such as mammalian sterile twenty-like protein (MST1), MEKK1, ASK1 and SLK, and those which are indirectly activated as a result of the kinase activity of a protein that has been directly activated by caspase-3, for example MKK6, MKK3, p38α and p38γ.

As is known in the art, caspase activation of proteins typically involves a cleavage step that results in an active form of the protein. For example, caspase-3 activation of MST1 is proposed to take place by cleavage at the motif present between amino acid residues 323-327 (see, Juo, et al., Mol. Cell. Biol. (1997) 17:24-35). Thus, the present invention also contemplates the use of active forms of the above proteins, such as the active form of MST1 comprising amino acids 1 to 327.

Biologically active fragments are fragments of the naturally occurring (or wild-type) protein that retain substantially the same activity as the wild-type protein and include activated forms of proteins as described above as well as smaller or larger fragments that retain substantially the same activity. An example of an active fragment of a protein is provided by the fragment of MST1 comprising amino acids 1 to 330 (see Kolodziejczyk, et al., Curr. Biol. (1999) 9:1203-1206, and Example 1 herein).

Candidate fragments can be selected from random fragments generated from the wild-type protein or can be specifically designed. The activity of the fragments is tested and compared to that of the wild-type protein and those fragments with substantially the same activity as the wild-type protein are selected. Methods for generating polypeptide fragments are well known in the art and include enzymatic, chemical or mechanical cleavage of the wild-type protein or a recombinant version thereof, expression of polynucleotides encoding such fragments, and the like.

In accordance with the present invention, the proteins and polypeptides are prepared in such a manner that their intrinsic activity is retained. One skilled in the art will understand that amino acid residues may be deleted, added or substituted for those that appear in the amino acid sequences of the wild-type protein to produce a variant protein or fragment which retains substantially the same biological activity as the wild-type protein. Such variant proteins and fragments are considered to be within the scope of the present invention.

In the context of the present invention, a variant protein or a protein fragment is considered to have substantially the same activity as the wild-type protein when it exhibits 50% of the activity of the wild-type protein. In one embodiment, the variant protein or fragment exhibits 60% of the activity of the wild-type protein. In another embodiment, the variant protein or fragment exhibits 75% of the activity of the wild-type protein. In still another embodiment, the variant protein or fragment exhibits 90% of the activity of the wild-type protein.

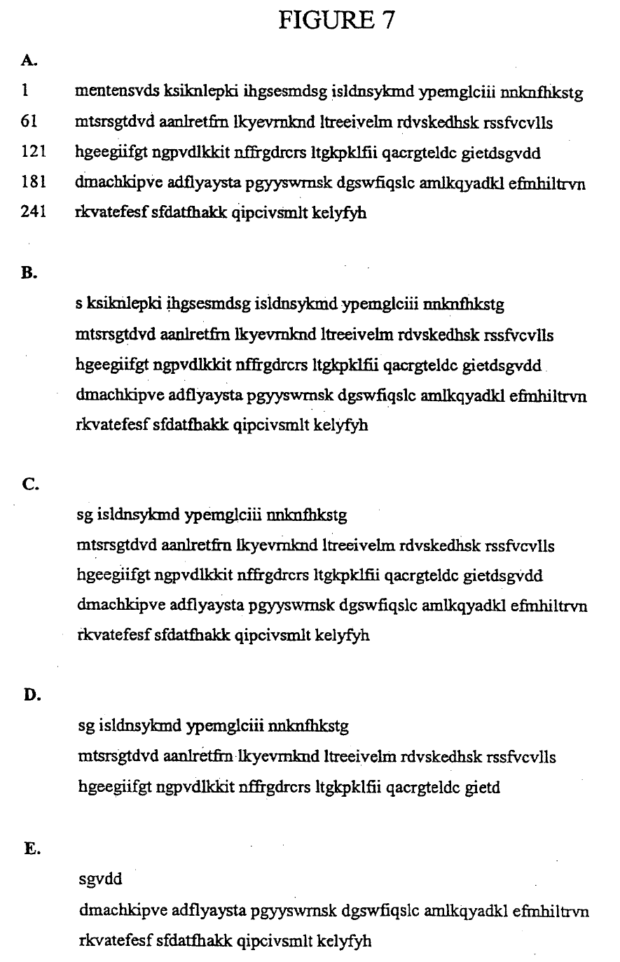

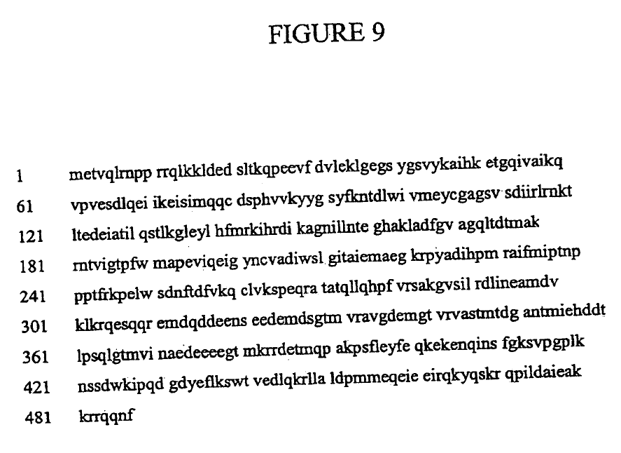

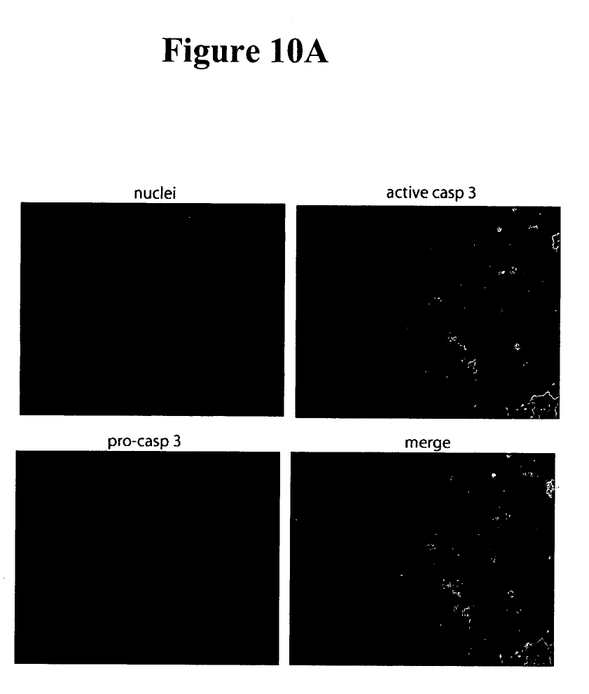

The proteins and polypeptides of the present invention can be prepared by methods known in the art, such as purification from cell extracts or the use of recombinant techniques (see, for example, Colligan et al. Current Protocols in Protein Science, John Wiley & Sons, New York; Ausubel et al. (1994 & updates) Current Protocols in Molecular Biology, John Wiley & Sons, New York). The amino-acid sequences of many of the proteins involved in the caspase-3 signalling pathway are known in the art, for example, caspase-3 [Genbank Accession Nos. gi|857569, NP_{—}033940 (mouse) and AAA74929 (human)] (see also FIG. 7), MST1 [Genbank Accession No. gi|1117791] (see FIG. 9), MEKK1 [Genbank Accession No. gi|2815888], ASK1 [Genbank Accession No. gi|1805500], SLK [Genbank Accession Nos. AAD28717 (mouse) and gi|7661994 (human, putative)], MKK6 [Genbank Accession Nos. NP_{—}036073 (mouse), NP_{—}114365 and NP_{—}002749 (human)], MKK3 [Genbank Accession Nos. NP_{—}032954 (mouse) and NP_{—}659732 (human)], p38α [Genbank Accession Nos. NP_{—}036081 (mouse) and NP_{—}001306 (human)] and p38γ [Genbank Accession Nos. NP_{—}002960 (human) and NP_{—}038899 (mouse)].

Polypeptides derived from one of these sequences, or fragments thereof, can also be chemically synthesised by methods known in the art including, but not limited to, exclusive solid phase synthesis, partial solid phase synthesis, fragment condensation or classical solution synthesis (Merrifield (1963) J. Am. Chem. Soc. 85:2149; Merrifield (1986) Science 232:341). The proteins and polypeptides of the present invention can be purified using standard techniques such as chromatography (e.g. ion exchange, affinity, and sizing column chromatography or high performance liquid chromatography), centrifugation, differential solubility, or by other techniques familiar to a worker skilled in the art.

1.2 Polynucleotides

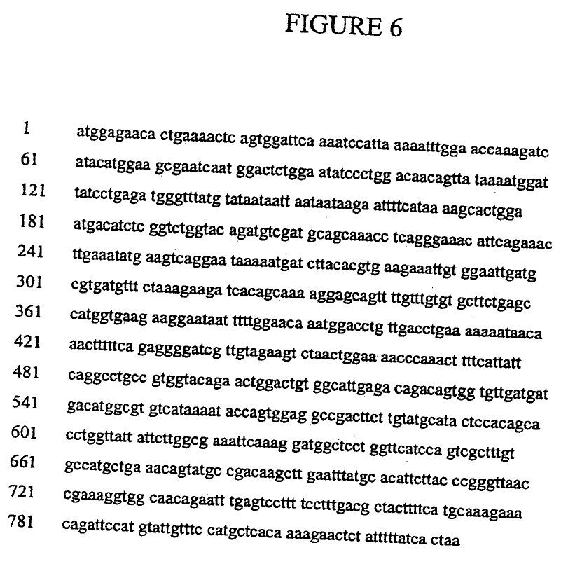

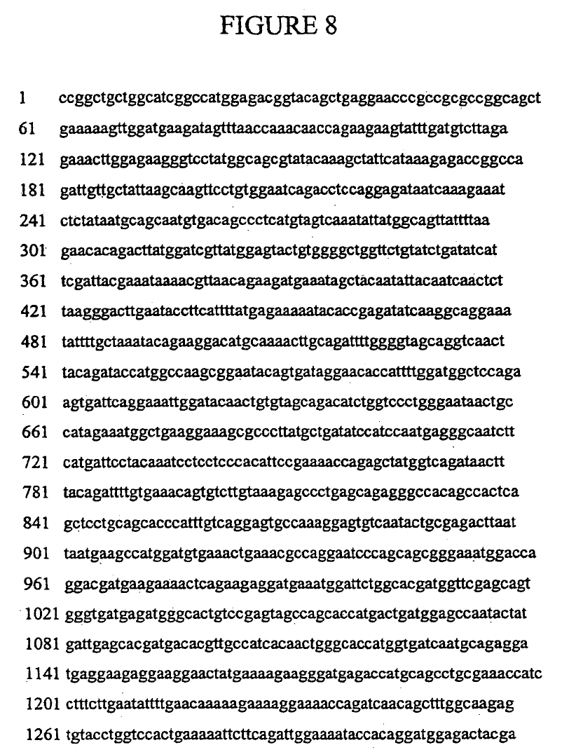

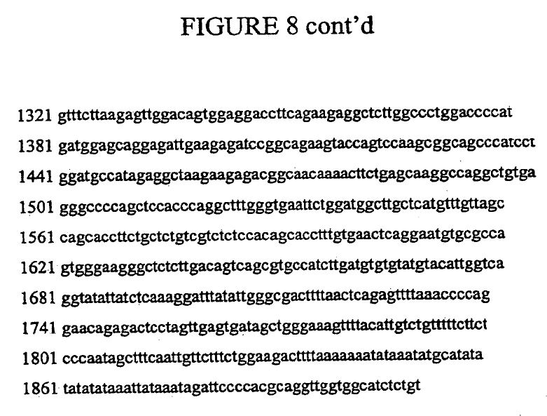

In one embodiment of the present invention, the proteins and polypeptides are produced by recombinant techniques. Such techniques are well known in the art (see, for example, Sambrook et al., (2000) Molecular Cloning: A Laboratory Manual, Cold Spring Harbor Laboratory Press, NY; Ausubel et al. (1994 & updates) Current Protocols in Molecular Biology, John Wiley & Sons, New York) and typically involve transformation (including transfection, transduction, or infection) of a suitable host cell with an expression vector comprising all or part of a DNA encoding the protein or polypeptide. The nucleotide sequences for caspase-3 gene isolated from human and mouse are known in the art (Tewari et al. (1995) Cell 81:801-809; GenBank Accession Nos. gi|857568 and U26943 (human) and NM_{—}009810 (mouse); see also FIG. 6), as is the nucleotide sequence for MST1 (Creasey and Chernoff (1995) J. Biol. Chem. 260:21695-21700; GenBank Accession No. gi|1117790; see FIG. 8). The gene sequences for a number of other proteins involved in the caspase-3 signalling pathway are known, for example, MEKK1 [Genbank Accession Nos. gi|2815887, NM_{—}011943 (mouse), NM_{—}031988 and NM_{—}002758 (human)], ASK1 [Genbank Accession No. gi|1805499], SLK [Genbank Accession Nos. gi|4741822 and AF112855 (mouse) and gi|7661994 (human, putative)], MKK6 [Genbank Accession No. gi|1209670], MKK3 [Genbank Accession Nos. gi|685173, NM_{—}008928 (mouse) and NM_{—}145110 (human)], p38α [Genbank Accession Nos. gi|529039, NM_{—}011951 (mouse) and NM_{—}001315 (human)] and p38γ [Genbank Accession Nos. gi|1772645, NM_{—}002969 (human) and NM_{—}013871 (mouse)]. The above Accession Nos are provided by way of representative example only and are not intended to be limiting in any way. As will be readily appreciated by one skilled in the art, transcript variants of many of the above listed genes occur and are known in the art, for example, MKK6, MKK3, p38α and p38γ are all known to have transcript variants. Such transcript variants are also encompassed within the scope of the present invention.

Polynucleotides encoding a protein or polypeptide according to the present invention can be readily purified from a suitable source by standard techniques. The polynucleotides can be genomic DNA or RNA or they can be cDNA prepared from isolated mRNA by standard techniques. Suitable sources for obtaining the polynucleotides are those cells which are known to express caspase-3 and other proteins in the caspase-3 signalling cascade, such as myocytes, hepatocytes, thymocytes, cardiomyocytes, neural cells and the like. In addition, polynucleotides encoding proteins in the caspase-3 signalling cascade including caspase-3 and MST-1 are commercially available (for example, from clone bank collections available from Stratagene (La Jolla, Calif.).

Suitable expression vectors for use with the polynucleotides of the present invention include, but are not limited to, plasmids, phagemids, viral particles and vectors, phage and the like. For insect cells, baculovirus expression vectors are suitable. The entire expression vector, or a part thereof, can be integrated into the host cell genome. In some circumstances, it is desirable to employ an inducible expression vector, e.g., the LACSWITCH™ Inducible Expression System (Stratagene, LaJolla, Calif.). In one embodiment of the present invention, the expression vector pcDNA3.1 Myc/His (Invitrogen, Carlsbad, Calif.) is used.

Those skilled in the field of molecular biology will understand that a wide variety of expression systems can be used to provide the recombinant protein or polypeptide. The precise host cell used is not critical to the invention. The protein or polypeptide can be produced in a prokaryotic host (e.g., E. coli or B. subtilis) or in a eukaryotic host (e.g., Saccharomyces or Pichia; mammalian cells, such as COS, NIH 3T3, CHO, BHK, 293, or HeLa cells; or insect cells). Proteins and polypeptides can also be produced using plant cells. For plant cells viral expression vectors (such as cauliflower mosaic virus and tobacco mosaic virus) and plasmid expression vectors (such as the Ti plasmid) are suitable. The methods of transformation or transfection and the choice of expression vector will depend on the host system selected and can be readily determined by one skilled in the art. Transformation and transfection methods are described, for example, in Ausubel et al. (1994) Current Protocols in Molecular Biology, John Wiley & Sons, New York; and various expression vectors may be chosen from those provided, e.g., in Cloning Vectors: A Laboratory Manual (Pouwels et al., 1985, Supp. 1987).

The host cells harbouring the expression vehicle can be cultured in conventional nutrient media adapted as needed for activation of a chosen gene, repression of a chosen gene, selection of transformants, or amplification of a chosen gene.

It will be apparent to one skilled in the art that the polynucleotides encoding the wild-type proteins can be altered by standard techniques, such as site-directed mutagenesis, such that nucleic acids are deleted from, added to, and/or substituted within, the coding sequence and an active protein or polypeptide is still expressed. Nucleic acid variants that encode a protein or polypeptide with substantially the same activity as the wild-type protein are considered to be within the scope of the present invention.

Furthermore, the proteins and polypeptides of the present invention can be produced as fusion proteins. One use of such fusion proteins is to improve the purification or detection of the protein or polypeptide. For example, a protein or polypeptide can be fused to an immunoglobulin Fc domain and the resultant fusion protein can be readily purified using a protein A column. Other examples of fusion proteins include proteins or polypeptides fused to histidine tags (allowing for purification on Ni²⁺ resin columns), to glutathione-S-transferase (allowing purification on glutathione columns) or to biotin (allowing purification with streptavidin labelled magnetic beads).

Specific initiation signals may be required for efficient translation of cloned polynucleotides. These signals include the ATG initiation codon and adjacent sequences. In cases where an entire wild-type gene or cDNA, including its own initiation codon and adjacent sequences, is inserted into the appropriate expression vector, no additional translational control signals may be needed. In other cases, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators (Bittner et al. (1987) Methods in Enzymol. 153, 516).

In addition, as would be readily appreciated by a worker skilled in the art, a host cell may be chosen which modulates the expression of the inserted sequences, or modifies and processes the gene product in a specific, desired fashion. Such modifications (e.g., glycosylation) and processing (e.g., cleavage) of protein products may be important for the activity of the protein. Different host cells have characteristic and specific mechanisms for the post-translational processing and modification of proteins and gene products. Appropriate cell lines or host systems can be chosen by one skilled in the art to ensure the correct modification and processing of the foreign protein expressed.

In one embodiment of the present invention, the polynucleotide encoding the protein/polypeptide is introduced directly into the stem cell. One skilled in the art will appreciate that for this particular application, the choice of expression vector is more important. Suitable vectors for this purpose are known in the art and are typically viral-based vectors. Representative examples are also described herein (see, Gene Therapy section and Examples).

1.3 Other Compounds

Candidate compounds that can be screened for their ability to act as activators or effectors of caspase-3 can be randomly selected or rationally selected or designed. As used herein, a candidate compound is said to be randomly selected when the compound is chosen randomly without considering the specific interactions involved in its potential association with molecular components of the stem cells, or other cells if co-culture is used. An example of random selection of candidate compounds is the use a chemical library or a peptide combinatorial library, or a growth broth of an organism. As used herein, a candidate compound is said to be rationally selected or designed when the compound is chosen on a non-random basis which takes into account the sequence and or conformation of a target site or a process in connection with the compound's action. Candidate compounds can be rationally selected or rationally designed, for example, by using the nucleotide or peptide sequences that make up the target sites. For example, a rationally selected peptide can be a peptide whose amino acid sequence is identical to or a derivative of a functional consensus site.

It is contemplated that the candidate compounds of the present invention can be, for example, polynucleotides, small molecules (including polypeptides), antibodies (and/or fragments thereof), synthesised organic molecules, naturally occurring organic molecules, vitamin derivatives, carbohydrates, and components or derivatives thereof.

The candidate compound may be isolated or unisolated, pure, partially purified, or in the form of a crude mixture, for example, it may be in the form of a cell, a lysate or extract derived from a cell, or a molecule derived from a cell. Where the candidate compound is present in a composition that comprises more than one molecular entity, it is contemplated that the composition may be tested as is and/or may optionally be fractionated by a suitable procedure and the fractionated sample tested using the method of the invention or another method to identify a particular fraction or component of the composition that acts as an activator or effector of caspase-3. It is further contemplated that sub-fractions of test compositions may be re-fractionated and assayed repeatedly using the methods of the invention with the ultimate goal of excluding inactive components from the sub-combination identified as an activator or effector of caspase-3. Intervening steps of compound isolation, purification and/or characterisation may be included as needed or appropriate before, during and/or after a method of the invention is employed.

Candidate compounds can be obtained in the form of large libraries of synthetic or natural compounds. Numerous means are currently used for random and directed synthesis of saccharide, peptide, and nucleic acid based compounds and are well-known in the art. Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries are also available or can be prepared according to standard procedures. Alternatively, libraries of natural compounds in the form of bacterial, fungal, plant, and animal extracts are available from, for example, Pan Laboratories (Bothell, Wash.) or MycoSearch (North Carolina), or can be readily produced. Additionally, natural and synthetically produced libraries and compounds are readily modified through conventional chemical, physical, and biochemical means.

2. Inhibitors of Caspase-3

In the context of the present invention, an inhibitor is a compound that directly decreases the activity of caspase-3 by acting on the caspase-3 protein or gene, or that indirectly decreases the activity or effect of caspase-3 by acting upstream or downstream of caspase-3 in the caspase-3 signalling pathway. That is, an inhibitor attenuates or inactivates a part of the cascade of molecular and cellular events that leads to or results from caspase-3 activation.

Examples of inhibitors encompassed by the present invention include, but are not limited to, oligonucleotide inhibitors (e.g. antisense molecules), antibodies, biologically inactive fragments or variants of the caspase-3 protein; biologically inactive fragments or variants of proteins that are activated by caspase-3 in the signalling pathway; proteins that are inactivated by caspase-3 in the signalling pathway or biologically active fragments or variants thereof; biologically inactive fragments or variants of proteins that activate caspase-3 in the signalling pathway; proteins that inhibit caspase-3 in the signalling pathway or biologically active fragments or variants thereof; nucleic acid sequences encoding such proteins, fragments or variants, and other compounds that decrease the activity of caspase-3 or reduce expression of caspase-3.

Proteins that are activated by caspase-3 in the signalling pathway include those described above. Proteins that inhibit caspase-3 activity include those that directly inhibit caspase-3, such as XIAP, c-IAP2, c-IAP1 and survivin, and those which limit caspase-3 activity indirectly by inhibition of caspase-3 activating proteins (such as caspase-1, caspase-8, caspase-9, caspase-10 and granzyme B) including, but not limited to, I-FLICE, CrmA, XIAP, c-IAP2, c-IAP1 and survivin.

2.1 Oligonucleotide Inhibitors

The oligonucleotide inhibitors according to the present invention are targeted to a mammalian caspase-3 gene or other a gene encoding a protein activated by caspase-3, such as MST1, or a gene encoding a protein that activates caspase-3. The nucleotide sequences for genes encoding proteins that act downstream or upstream of caspase-3 in the signalling pathway are known in the art and a number are provided supra (section 1.2). Other known sequences include, for example, p38 (GenBank Accession No. gi|1772645), p38 (GenBank Accession No. gi|529039), XIAP (GenBank Accession No. gi|8923794), c-IAP2 (GenBank Accession No. gi|3978243), c-IAP1 (GenBank Accession No. gi|14770186), survivin (GenBank Accession No. gi|4502144), caspase-1 (GenBank Accession No. gi|537291), caspase-8 (GenBank Accession No. gi|2429161), caspase-9 (GenBank Accession No. gi|3056726), caspase-10 (GenBank Accession No. gi|3386522), granzyme B (GenBank Accession No. gi|1247450), I-FLICE (GenBank Accession No. gi|2827289) and CrmA (GenBank Accession No. gi|323401).

In one embodiment of the present invention, the oligonucleotide inhibitors are targeted to a mammalian caspase-3 gene. In another embodiment, they are targeted to the human caspase-3 gene. In another embodiment, they are targeted to a mammalian MST1 gene. In another embodiment, they are targeted to the human MST1 gene.

In the context of the present invention, the term “oligonucleotide inhibitor” encompasses antisense oligonucleotides, short interfering RNA (siRNA) molecules, ribozymes and triple helix-forming oligonucleotides.

The term “oligonucleotide” as used herein refers to an oligomer or polymer of ribonucleic acid (RNA), deoxyribonucleic acid (DNA), or modified versions thereof, or RNA or DNA mimetics. This term, therefore, includes oligonucleotides composed of naturally occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for the nucleic acid target and increased stability in the presence of nucleases. The term also includes chimeric oligonucleotides. Chimeric oligonucleotides are oligonucleotides that contain two or more chemically distinct regions, each region comprising at least one monomer unit. The oligonucleotides according to the present invention can be single-stranded or they can be double-stranded.

As is known in the art, a nucleoside is a base-sugar combination and a nucleotide is a nucleoside that further includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Specific examples of oligonucleotides useful in this invention include those containing modified backbones or non-natural internucleoside linkages. As defined in this specification, oligonucleotides having modified backbones include both those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary modified oligonucleotide backbones that can be incorporated into the oligonucleotides according to the present invention include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogues of these, and analogues having inverted polarity wherein the adjacent pairs of nucleoside units are linked 3′-5′ to 5′-3′ or 2′-5′ to 5′-2′. Various salts, mixed salts and free acid forms are also included.

Exemplary modified oligonucleotide backbones that do not include a phosphorus atom include those formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include morpholino linkages (formed in part from the sugar portion of a nucleoside); siloxane backbones; sulphide, sulphoxide and sulphone backbones; formacetyl and thioformacetyl backbones; methylene formacetyl and thioformacetyl backbones; alkene containing backbones; sulphamate backbones; methyleneimino and methylenehydrazino backbones; sulphonate and sulphonamide backbones; amide backbones; and others having mixed N, O, S and CH₂ component parts.

The term “alkyl” as used herein refers to monovalent alkyl groups having from 1 to 20 carbon atoms. In one embodiment of the present invention the alkyl group has between 1 and 6 carbon atoms. Examples of suitable alkyl groups include, but are not limited to, methyl, ethyl, n-propyl, iso-propyl, n-butyl, iso-butyl, n-hexyl, and the like.

The term “cycloalkyl” refers to cyclic alkyl groups of from 3 to 20 carbon atoms having a single cyclic ring or multiple condensed rings. Examples of suitable cycloalkyl groups include, but are not limited to, single ring structures such as cyclopropyl, cyclobutyl, cyclopentyl, cyclooctyl, and the like, or multiple ring structures such as adamantanyl, and the like.

The present invention also contemplates oligonucleotide mimetics in which both the sugar and the internucleoside linkage of the nucleotide units are replaced with novel groups. The base units are maintained for hybridization with an appropriate nucleic acid target. An example of such an oligonucleotide mimetic; which has been shown to have excellent hybridization properties, is a peptide nucleic acid (PNA) [Nielsen et al., Science, 254:1497-1500 (1991)]. In PNA compounds, the sugar-backbone of an oligonucleotide is replaced with an amide-containing backbone, in particular an aminoethylglycine backbone. The nucleobases are retained and are bound directly or indirectly to aza-nitrogen atoms of the amide portion of the backbone.

Modified oligonucleotides according to the present invention may also contain one or more substituted sugar moieties. For example, oligonucleotides may comprise sugars with one of the following substituents at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Examples of such groups are: O[(CH₂)ₙ O]ₘ CH₃, O(CH₂)ₙ OCH₃, O(CH₂)ₙ NH₂, O(CH₂)ₙ CH₃, O(CH₂)ₙ ONH₂, and O(CH₂)ₙ ON[(CH₂)ₙ CH₃)]₂, where n and m are from 1 to about 10. Alternatively, the oligonucleotides may comprise one of the following substituents at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Specific examples include 2′-O-methyl (2′-O—CH₃), 2′-methoxyethoxy (2′-O—CH₂ CH₂ OCH₃, also known as 2′-O-(2-methoxyethyl) or 2′-MOE) [Martin et al., Helv. Chim. Acta, 78:486-504(1995)], 2′-dimethylaminooxyethoxy (2′-O(CH₂)₂ ON(CH₃)₂ group, also known as 2′-DMAOE), 2′-aminopropoxy (2′-OCH₂ CH₂ CH₂ NH₂) and 2′-fluoro (2′-F).

Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also comprise sugar mimetics such as cyclobutyl moieties in place of the pentofuranosyl sugar.

Oligonucleotides according to the present invention may also include modifications or substitutions to the nucleobase. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C); inosine; 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioalkyl, 8-hydroxyl and other 8-substituted adenines and guanines; 5-halo particularly 5-bromo, 5-trifluoromethyl and other 5-substituted uracils and cytosines; 7-methylguanine and 7-methyladenine; 8-azaguanine and 8-azaadenine; 7-deazaguanine and 7-deazaadenine; 3-deazaguanine and 3-deazaadenine. Further nucleobases include those disclosed in U.S. Pat. No. 3,687,808; The Concise Encyclopaedia Of Polymer Science And Engineering, (1990) pp 858-859, Kroschwitz, J. I., ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi, Y. S., (1993) Antisense Research and Applications, pp 276-278, Crooke, S. T. and Lebleu, B., ed., CRC Press, Boca Raton].

Another oligonucleotide modification included in the present invention is the chemical linkage to the oligonucleotide of one or more moieties or conjugates which enhance the activity, cellular distribution or cellular uptake of the oligonucleotide. Such moieties include, but are not limited to, lipid moieties such as a cholesterol moiety [Letsinger et al., Proc. Natl. Acad. Sci. USA, 86:6553-6556 (1989)], cholic acid [Manoharan et al., Bioorg. Med. Chem. Let., 4:1053-1060 (1994)], a thioether, e.g. hexyl-S-tritylthiol [Manoharan et al., Ann. N.Y. Acad. Sci., 660:306-309 (1992); Manoharan et al., Bioorg. Med. Chem. Lett., 3:2765-2770 (1993)], a thiocholesterol [Oberhauser et al., Nucl. Acids Res., 20:533-538 (1992)], an aliphatic chain, e.g. dodecandiol or undecyl residues [Saison-Behmoaras et al., EMBO J., 10:1111-1118 (1991); Kabanov et al., FEBS Lett., 259:327-330 (1990); Svinarchuk et al., Biochimie, 75:49-54 (1993)], a phospholipid, e.g. di-hexadecyl-rac-glycerol or triethylammonium 1,2-di-O-hexadecyl-rac-glycero-3-H-phosphonate [Manoharan et al., Tetrahedron Lett., 36:3651-3654 (1995); Shea et al., Nucl. Acids Res., 18:3777-3783 (1990)], a polyamine or a polyethylene glycol chain [Manoharan et al., Nucleosides & Nucleotides, 14:969-973 (1995)], or adamantane acetic acid [Manoharan et al., Tetrahedron Lett., 36:3651-3654 (1995)], a palmityl moiety [Mishra et al., Biochim. Biophys. Acta, 1264:229-237 (1995)], or an octadecylamine or hexylamino-carbonyl-oxycholesterol moiety [Crooke et al., J. Pharmacol. Exp. Ther., 277:923-937 (1996)].

One skilled in the art will recognise that it is not necessary for all positions in a given oligonucleotide to be uniformly modified. The present invention, therefore, contemplates the incorporation of more than one of the aforementioned modifications into a single oligonucleotide or even at a single nucleoside within the oligonucleotide.

As indicated above, oligonucleotides that are chimeric compounds are included within the scope of the present invention. Chimeric oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids.

In the context of the present invention, an oligonucleotide is “nuclease resistant” when it has either been modified such that it is not susceptible to degradation by DNA and RNA nucleases or, alternatively, has been placed in a delivery vehicle which itself protects the oligonucleotide from DNA or RNA nucleases. Nuclease resistant oligonucleotides include, for example, methyl phosphonates, phosphorothioates, phosphorodithioates, phosphotriesters, and morpholino oligomers. Suitable delivery vehicles for conferring nuclease resistance include, for example, liposomes.

The present invention further contemplates oligonucleotides that contain groups for improving the pharmacokinetic properties of the oligonucleotide, or groups for improving the pharmacodynamic properties of the oligonucleotide.

2.1.1 Antisense Oligonucleotides

The term “antisense oligonucleotide” as used herein indicates an oligonucleotide having a nucleotide sequence that is complementary to a portion of the mRNA transcribed from a gene of interest (i.e. a gene that encodes a protein of interest such as a protein in the caspase-3 signalling pathway).

The antisense oligonucleotides according to the present invention are targeted to a gene of interest. “Targeting” an antisense oligonucleotide to a particular nucleic acid, in the context of the present invention, is a multistep process that usually begins with the identification of a nucleic acid sequence whose function is to be modulated. In the context of the present invention the target is a gene encoding a protein in the caspase-3 signalling pathway or the mRNA transcribed therefrom. The targeting process also includes determination of a site, or sites, within this nucleic acid sequence for the antisense interaction to occur such that modulation of expression of the protein encoded by the gene will result. Once the target site, or sites, has been identified, oligonucleotides are chosen that are sufficiently complementary (i.e. that hybridise with sufficient strength and specificity) to the target to give the desired result.

Generally, there are five regions of a gene, or mRNA transcribed therefrom, that may be targeted for antisense modulation: the 5′ untranslated region (5′-UTR), the translation initiation (or start) codon region, the open reading frame (ORF), the translation termination (or stop) codon region and the 3′ untranslated region (3′-UTR).

The terms “translation initiation codon” and “start codon” can encompass many codon sequences, even though the initiator amino acid in each instance is typically methionine (in eukaryotes). It is also known in the art that eukaryotic genes may have two or more alternative start codons, any one of which may be preferentially utilised for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding the protein of interest regardless of the sequence(s) of such codons.

As is known in the art, some eukaryotic transcripts are directly translated, however, most mammalian genes, or open reading frames (ORFs), contain one or more sequences, known as “introns,” which are excised from a transcript before it is translated. The expressed (unexcised) portions of the ORF are referred to as “exons” and are spliced together to form an mRNA transcript (Alberts et al., (1983) Molecular Biology of the Cell, Garland Publishing Inc., New York, pp. 411-415). In the context of the present invention, both introns and exons may serve as targets for antisense. In some instances, an ORF may also contain one or more sites that may be targeted for antisense due to some functional significance in vivo. Examples of the latter types of sites include intragenic stem-loop structures (see, for example, U.S. Pat. No. 5,512,438) and, in unprocessed mRNA molecules (primary transcripts), intron/exon splice sites. As is known in the art, primary RNA transcripts can be alternatively processed in vivo depending on the splicing of the exons and can, therefore, give rise to alternatively spliced mRNA molecules which correspond to the same gene but differ in structure. In addition, mRNA molecules possess a 5′ cap region that may also serve as a target for antisense. The 5′ cap of a mRNA comprises an N⁷-methylated guanosine residue joined to the 5′-most residue of the mRNA via a 5′-5′ triphosphate linkage. The 5′ cap region of a mRNA is considered to include the 5′ cap structure itself as well as the first 50 nucleotides adjacent to the cap.

Thus, the antisense oligonucleotides according to the present invention can be complementary to regions of the complete gene including the introns, to the primary mRNA transcript of the gene or to one or more of the final, spliced versions of the mRNA transcribed from the gene of interest.

The antisense oligonucleotides in accordance with the present invention are selected from a sequence complementary to a gene of interest such that the sequence exhibits the least likelihood of forming duplexes, hair-pins, or of containing homooligomer/sequence repeats. The oligonucleotide may further contain a GC clamp. One skilled in the art will appreciate that these properties can be determined qualitatively using various computer modelling programs, for example, the program OLIGO® Primer Analysis Software, Version 5.0 (distributed by National Biosciences, Inc., Plymouth, Minn.).

Alternatively, antisense oligonucleotides can be selected that are complementary to a nucleic acid sequence which constitutes a region of a target gene that is highly conserved between the genes of two or more species. These properties can be determined, for example, using the BLASTN program (Altschul, et al., (1990) J. Mol. Biol., 215:403-10) of the University of Wisconsin Computer group (GCG) software (Devereux. et al., (1984) Nucleic Acids Res., 12:387-395) with the National Center for Biotechnology Information (NCBI) databases.

It is understood in the art that an antisense oligonucleotide need not have 100% identity with the complement of its target sequence in order to be effective. The antisense oligonucleotides in accordance with the present invention, therefore, have a sequence that is at least about 70% identical to the complement of the target sequence. In one embodiment of the present invention, the antisense oligonucleotides have a sequence that is at least about 80% identical to the complement of the target sequence. In other embodiments, they have a sequence that is at least about 90% identical or at least about 95% identical to the complement of the target sequence, allowing for gaps or mismatches of several bases. Identity can be determined, for example, by using the BLASTN program of the University of Wisconsin Computer Group (GCG) software.

In order for the antisense oligonucleotides of the present invention to function in inhibiting expression of a gene of interest, it is necessary that they demonstrate adequate specificity for the target sequence and do not bind to other nucleic acid sequences in the cell. Therefore, in addition to possessing an appropriate level of sequence identity to the complement of the target sequence, the antisense oligonucleotides of the present invention should not closely resemble other known sequences. The antisense oligonucleotides of the present invention, therefore, should be less than 50% identical to any other mammalian nucleic acid sequence. The identity of the antisense oligonucleotides of the present invention to other sequences can be determined, for example, through the use of the BLASTN program and the NCBI databases as indicated above.

The antisense oligonucleotides according to the present invention are typically between 7 and 100 nucleotides in length. In one embodiment, the antisense oligonucleotides comprise from about 7 to about 50 nucleotides, or nucleotide analogues. In related embodiments the antisense oligonucleotides comprise from about 7 to about 35 nucleotides, or nucleotide analogues, and from about 15 to about 25 nucleotides, or nucleotide analogues.

In another embodiment of the present invention, the antisense oligonucleotides comprise one or more phosphorothioate backbone linkages. In a related embodiment, all backbone linkages in the antisense oligonucleotide are phosphorothioate linkages.

In yet another embodiment of the present invention, the antisense oligonucleotides are chimeric molecules with a mixed phosphorothioate and 2′-O-methyl backbone. In a related embodiment, the antisense oligonucleotides further comprise a TCCC motif. The presence of such a motif in an antisense oligonucleotide has been shown increase the likelihood that the mRNA:DNA duplex will undergo RNase H-mediated degradation. RNase H is a cellular endonuclease that cleaves the RNA strand of an RNA:DNA duplex. In antisense therapy, therefore, activation of RNase H results in cleavage of the mRNA target, thereby greatly enhancing the efficiency of oligonucleotide inhibition of gene expression.

2.1.2 Short Interfering RNA (siRNA) Molecules

RNA interference mediated by short interfering double-stranded RNA molecules (siRNA) is known in the art to play an important role in post-transcriptional gene silencing [Zamore, Nature Struc. Biol., 8:746-750 (2001)]. In nature, siRNA molecules are typically 21-22 base pairs in length and are generated when long double-stranded RNA molecules are cleaved by the action of an endogenous ribonuclease. Recently, it has been demonstrated that transfection of mammalian cells with synthetic siRNA molecules having a sequence identical to a target gene leads to a reduction in the mRNA levels of the target gene [Elbashir, et al., Nature, 411:494-498 (2001)].

The oligonucleotide inhibitors according to the present invention can be siRNA molecules that are targeted to a gene of interest such that the sequence of the siRNA corresponds to a portion of said gene. As is known in the art, effective siRNA molecules are typically less than 30 base pairs in length to help prevent them triggering non-specific RNA interference pathways in the cell via the interferon response. Thus, in one embodiment of the present invention, the siRNA molecules are between about 15 and about 25 base pairs in length. In a related embodiment, they are between about 19 and about 22 base pairs in length.

The double-stranded siRNA molecules can further comprise poly-T or poly-U overhangs at the 3′ and 5′ ends to minimise RNase-mediated degradation of the molecules. Thus, in another embodiment of the present invention, the siRNA molecules comprise overhangs at the 3′ and 5′ ends which comprise two thymidine or two uridine residues. Design and construction of siRNA molecules is known in the art [see, for example, Elbashir, et al., Nature, 411:494-498 (2001); Bitko and Barik, BMC Microbiol., 1:34 (2001)]. In addition, kits that provide a rapid and efficient means of constructing siRNA molecules by in vitro transcription are also commercially available (Ambion, Austin, Tex.; New England Biolabs, Beverly, Mass.) and may be used to construct the siRNA molecules according to the present invention.

2.1.3 Ribozymes

The oligonucleotide inhibitors according to the present invention can be ribozymes that specifically target mRNA encoding a protein of interest. As is known in the art, ribozymes are RNA molecules having an enzymatic activity that enables the ribozyme to repeatedly cleave other separate RNA molecules in a nucleotide-sequence specific manner. Such enzymatic RNA molecules can be targeted to virtually any mRNA transcript, and efficient cleavage can be achieved in vitro [Kim et al., Proc. Natl. Acad. Sci. USA, 84:8788, (1987); Haseloff and Gerlach, Nature, 334:585, (1988); Cech, JAMA, 260:3030, (1988); Jefferies et al., Nucleic Acids Res., 17:1371, (1989)].

Typically, a ribozyme comprises two portions held in close proximity: a mRNA binding portion having a sequence complementary to the target mRNA sequence, and a catalytic portion which acts to cleave the target mRNA. A ribozyme acts by first recognising and binding a target mRNA by complementary base-pairing through the target mRNA binding portion of the ribozyme. Once it is specifically bound to its target, the ribozyme catalyses cleavage of the target mRNA. Such strategic cleavage destroys the ability of a target mRNA to direct synthesis of an encoded protein. Having bound and cleaved its mRNA target, the ribozyme is released and can repeatedly bind and cleave new target mRNA molecules.

One of the best characterised ribozyme molecules is the “hammerhead ribozyme.” Hammerhead ribozymes comprise a hybridising region which is complementary in nucleotide sequence to at least part of the target mRNA, and a catalytic region which is adapted to cleave the target mRNA. In general, the hybridising region contains at least 9 nucleotides. The present invention therefore contemplates the use of the oligonucleotide inhibitors as part of the hybridising region of a hammerhead ribozyme, wherein the hybridising region comprises at least 9 nucleotides that are complementary to a gene encoding protein of interest and is joined to an appropriate catalytic domain. The construction and production of such ribozymes is well known in the art [see, for example, Haseloff and Gerlach, Nature, 334:585-591 (1988)].

Ribozymes in accordance with the present invention also include RNA endoribonucleases (hereinafter “Cech-type ribozymes”) such as the one which occurs naturally in Tetrahymena thermophila (known as the IVS, or L-19 IVS RNA), which has been extensively described in the literature [see, Zaug, et al., Science, 224:574-578 (1984); Zaug and Cech, Science, 231:470-475 (1986); Zaug, et al., Nature, 324:429433 (1986); U.S. Pat. No. 4,987,071; Been and Cech, Cell, 47:207-216 (1986)]. Cech-type ribozymes comprise an 8 nucleotide active site which hybridises to a target mRNA sequence with subsequent cleavage of the target mRNA by the ribozyme.

One skilled in the art will understand that there is a narrow range of binding free energies between a ribozyme and its substrate that will produce maximal ribozyme activity. Such binding energy can be optimized by making ribozymes with G to I (inosine) and U to BrU (bromouracil) substitutions (or equivalent substitutions as known in the art) in the mRNA binding portion. Such substitutions allow manipulation of the binding free energy without altering the target recognition sequence, the length of the mRNA binding portion, or the enzymatic portion of the ribozyme. The shape of the free-energy vs. ribozyme activity curve can be readily determined using data from standard experiments known in the art in which each base (or several bases) is modified or unmodified, and without the complication of changing the size of the ribozyme/substrate interaction.

If necessary, such experiments can be used to indicate the most active ribozyme structure. The use of modified bases thus permits “fine tuning” of the binding free energy to assure maximal ribozyme activity and is considered to be within the scope of the present invention. In addition, replacement of such bases, e.g. I for G, may permit a higher level of substrate specificity when cleavage of non-target RNA is a problem.

2.1.4 Triple Helix-Forming Oligonucleotides

In another embodiment of the present invention the inhibitor is an oligonucleotide that hybridises to and forms triple helix structures at the 5′ terminus of the target gene and can thus be used to block transcription. The triple helix forming oligonucleotides can be prepared as described above in relation to antisense oligonucleotides.

2.1.5 Efficacy of the Oligonucleotide Inhibitors

The efficacy of the oligonucleotide inhibitors according to the present invention in inhibiting the expression of a gene of interest can be determined using a number of different methods known in the art. Exemplary methods that can be used to determine the efficacy of the oligonucleotides of the present invention are provided below.

Initial determinations of the efficacy of the oligonucleotides of the present invention can be made using in vitro techniques. For example, the oligonucleotide can be introduced into a cell line that normally expresses or overexpresses the targeted gene and the amount of mRNA transcribed from the gene can be measured by standard techniques, such as Northern blot analysis or quantitative RT-PCR. Alternatively, the amount of targeted protein produced by the cell can be measured, for example by Western blot analysis. The amount of mRNA or protein produced in a cell treated with the oligonucleotide can then be compared against the amount produced in a control, untreated cell or a cell treated with a control oligonucleotide and provides an indication of how successfully the oligonucleotide has inhibited gene expression. The specificity of the oligonucleotides for their mRNA target can be determined by conducting appropriate control experiments in parallel. Appropriate controls will be dependent upon the type of oligonucleotide inhibitor being investigated and can be readily selected by one skilled in the art. Examples of appropriate controls include untreated cells and cells treated with randomised or “scrambled” oligonucleotides, oligonucleotides containing a defined number of mismatches, long non-specific double-stranded RNA molecules or ribozymes incorporating a randomised oligonucleotide in its mRNA binding domain. Methods of conducting these methods are well-known to workers skilled in art (see, for example, Ausubel et al., (2000) Current Protocols in Molecular Biology, Wiley & Sons, New York: Coligan, et al., (2001) Current Protocols in Protein Science, Wiley & Sons, New York).

Alternatively, the ability of the oligonucleotide inhibitors of the invention to inhibit the expression of a target protein can be determined in vitro by assaying the total cellular activity of the protein in the presence and absence of the oligonucleotide. When the protein is an enzyme, the level of the protein is determined by measuring a level of enzymatic activity associated with that protein, for example by measuring the inhibitor-induced change in cellular levels of substrates or products of reactions catalysed by the enzyme. One skilled in the art will recognise that a variety of cell lines, reporter genes and detection methods known in the art can be used in this type of assay.

The ability of the oligonucleotide inhibitors to reduce target protein expression or activity or otherwise promote stem cell proliferation or decrease differentiation can also be determined explant cultures or in an appropriate animal models. For example, appropriate concentrations of oligonucleotide inhibitors can be incubated with neurosphere cultures (see Hitoshi et al. (2002) Genes & Dev. 16, 846-858) or intact skeletal muscle fibre cultures (Asakura et al. (2002) J. Cell. Biol 159, 123-134) and examined for the ability to inhibit stem cell growth from these explants. Similarly, the oligonucleotide inhibitors can be incubated with cardiac stem cell co-cultures using primary cardiomyocytes as educator cells (see, for example, Hierlihy et al. (2002) FEBS Lett. 530, 239-243) and assessed for their ability to inhibit proliferation.

Toxicity of the oligonucleotide inhibitors can also be initially assessed in vitro using standard techniques. For example, human primary fibroblasts can be treated in vitro with the oligonucleotide in the presence of a commercial lipid carrier such as lipofectamine. Cells are then tested at different time points following treatment for their viability using a standard viability assay, such as the trypan-blue exclusion assay. Cells are also assayed for their ability to synthesise DNA, for example, using a thymidine incorporation assay, and for changes in cell cycle dynamics, for example, using a standard cell sorting assay in conjunction with a fluorocytometer cell sorter (FACS).

In vivo toxic effects of the oligonucleotides can be evaluated by methods known in the art, for example, by measuring their effect on animal body weight during treatment and by performing haematological profiles and liver enzyme analysis after the animal has been sacrificed.

2.1.6 Preparation of the Oligonucleotide Inhibitors

The oligonucleotide inhibitors of the present invention can be prepared by conventional techniques well-known to those skilled in the art. For example, the oligonucleotides can be prepared using solid-phase synthesis using commercially available equipment, such as the equipment available from Applied Biosystems Canada Inc. (Mississauga, Canada). As is well-known in the art, modified oligonucleotides, such as phosphorothioates and alkylated derivatives, can also be readily prepared by similar methods.

Alternatively, the oligonucleotide inhibitors of the present invention can be prepared by enzymatic digestion and/or amplification of the naturally occurring target gene or mRNA, or of cDNA synthesized from the mRNA, using standard techniques known in the art. When the oligonucleotide inhibitors comprise RNA, they can be prepared by in vitro transcription methods also known in the art. As indicated above, siRNA molecules can also be conveniently prepared using commercially available in vitro transcription kits.

Oligonucleotides can also be prepared using recombinant DNA techniques. The present invention, therefore, encompasses expression vectors comprising nucleic acid sequences that encode the oligonucleotide inhibitors and subsequent expression of the encoded oligonucleotides in a suitable host cell. Such expression vectors can be readily constructed using procedures known in the art [see, for example, Ausubel, et al., Current Protocols in Molecular Biology, John Wiley & Sons, Inc, NY. (1989 and updates)], such as those described supra (see Section 1.2).

Ribozyme inhibitors according to the present invention can be readily constructed by techniques known in the art. In these molecules the oligonucleotide sequence is included in the hybridising or mRNA binding portion of the ribozyme and is joined to an appropriate catalytic portion. Selection of an appropriate catalytic portion is dependent on the type of ribozyme to be constructed and can be readily determined by one skilled in the art [see, for example, Haseloff and Gerlach, Nature, 334:585-591 (1988); U.S. Pat. No. 4,987,071].

2.2 Protein and Polypeptide Inhibitors

The proteins and polypeptide inhibitors of the present invention include, but are not limited to, biologically inactive fragments or variants of the caspase-3 protein; biologically inactive fragments or variants of proteins that are activated by caspase-3 in the signalling pathway; proteins that are inactivated by caspase-3 in the signalling pathway or biologically active fragments or variants thereof; biologically inactive fragments or variants of proteins that activate caspase-3 in the signalling pathway and proteins that inhibit caspase-3 in the signalling pathway or biologically active fragments or variants thereof.

Biologically active fragments are fragments of the wild-type protein that retain substantially the same activity as the wild-type protein. Exemplary methods of producing such fragments are described supra (see Section 1.1). As indicated previously, a variant protein or a protein fragment is considered to have substantially the same activity as the wild-type protein when it exhibits about 50% of the activity of the wild-type protein.

The present invention also contemplates the use of a biologically inactive proteins or fragments of proteins that interfere with the action of the wild-type protein and thus, act as inhibitors of protein activity. Biologically inactive proteins or fragments contemplated by the present invention are those that have substantially less activity than the wild-type protein.

Candidate inhibitory fragments can be generated from the wild-type protein using standard methods. In the context of the present invention, a biologically inactive protein, fragment or variant is considered to have substantially less activity than the wild-type protein when it exhibits 75% or less of the activity of the wild-type protein. In another embodiment, the variant protein or fragment exhibits 60% or less of the activity of the wild-type protein. In still another embodiment, the biologically inactive variant protein or fragment exhibits about 50% or less of the activity of the wild-type protein, for example, between about 1% and about 40% of the activity of the wild-type protein.

The present invention also provides for peptides that bind to and inhibit the activity of caspase-3 or another protein in the caspase-3 signalling pathway. One exemplary method of identifying such peptides is by phage display techniques. Phage display libraries of random short peptides are commercially available, e.g. from New England Biolabs, Inc., and are utilised through an in vitro selection process known as “panning”. In its simplest form, panning involves first incubating the library of phage-displayed peptides with a plate, or bead, coated with the target molecule, then washing away unbound phage particles, and finally eluting the specifically bound phage. An example of a target molecule would be the caspase-3 protein, or a fragment thereof.

The peptide(s) displayed by the specifically-binding phage are then isolated and sequenced by standard techniques known to those skilled in the art. In some instances the binding strength of the isolated peptide is then tested using standard techniques.

Once a peptide has been identified, it may be used to prepare various peptide analogues, peptide derivatives and peptidomimetic compounds that share the same activity. Such compounds are well known in the art and may have advantages over naturally occurring peptides, including, for example, greater chemical stability, increased resistance to proteolytic degradation, enhanced pharmacological properties (such as, half-life, absorption, potency and efficacy), altered specificity (for example, a broad-spectrum of biological activities) and/or reduced antigenicity.

The protein and polypeptide inhibitors of the present invention can be prepared by methods known in the art as indicated above. Exemplary methods include purification from cell extracts, the use of recombinant techniques or by chemical synthesis.

2.3 Polynucleotides Encoding Protein and Polypeptide Inhibitors

In one embodiment of the present invention, the protein and polypeptide inhibitors are produced by recombinant techniques. Such techniques are well known in the art (see, for example, Sambrook et al., (2000) Molecular Cloning: A Laboratory Manual, Cold Spring Harbor Laboratory Press, NY; Ausubel et al. (1994 & updates) Current Protocols in Molecular Biology, John Wiley & Sons, New York) and are described generally supra (see Section 1.2).

2.4 Antibodies

The present invention also contemplates the use of antibodies, and antibody fragments, raised against a target protein in the caspase-3 signalling pathway and which can bind to and inhibit the protein. In the context of the present invention, a target protein would be a caspase-3 protein, a protein that activates caspase-3 or a protein that is activated by caspase-3 in the signalling pathway.

For the production of antibodies, various hosts including, for example, goats, rabbits, rats, mice and humans, can be immunised with the target protein, or with a fragment or peptide thereof that has immunogenic properties. Depending on the host species, various adjuvants may be used to increase the immunological response. Such adjuvants include, but are not limited to, Freund's adjuvant, mineral gels such as aluminium hydroxide, and surface active substances such as lysolecithin, pluronic polyols, polyanions, peptides, oil emulsions, Keyhole limpet hemolysin (KLH), and dinitrophenol. Examples of adjuvants used in humans include, for example, BCG (bacilli Calmette-Guerin) and Corynebacterium parvum.

The peptides or protein fragments used to induce antibodies can have an amino acid sequence consisting of as little as about 5 amino acids. In one embodiment of the present invention, amino acid sequences of at least about 10 amino acids are used. These peptides or protein fragments can be identical to a portion of the amino acid sequence of the wild-type protein or can contain the entire amino acid sequence of a small, naturally occurring molecule. If required, short stretches of amino acids of the target protein can be fused with those of another protein, such as KLH, and antibodies to the chimeric molecule can be produced.

Monoclonal antibodies to a target protein can be prepared using techniques that provide for the production of antibody molecules by continuous cell lines in culture. These include, but are not limited to, the hybridoma technique, the human B-cell hybridoma technique and the EBV-hybridoma technique (see, for example, Kohler, G. et al. (1975) Nature 256:495-497; Kozbor, D. et al. (1985) J. Immunol. Methods 81:31-42; Cote, R. J. et al. (1983) Proc. Natl. Acad. Sci. USA, 80:2026-2030; and Cole, S. P. et al. (1984) Mol. Cell Biol. 62:109-120). For example, the monoclonal antibodies according to the present invention can be obtained by immunising animals, such as mice or rats, with purified protein. Spleen cells isolated from the immunised animals are then immortalised using standard techniques.

Immortalization of the spleen cells from immunised animals can be carried out, for example, by fusing these cells with a myeloma cell line, such as P3X63-Ag 8.653 (ATCC CRL 1580), according to the method described in (1980) J. Imm. Meth. 39:285-308. Other methods known to a person skilled in the art can also be used to immortalise spleen cells. In order to detect immortalised cells that produce the desired antibody against the target protein, a sample of the culture supernatant is tested for reactivity using, for example, an enzyme linked immunosorbent assay (ELISA). In order to obtain those antibodies that inhibit the activity of the target protein, the culture supernatant of clones that produce antibodies that bind to the protein is additionally examined for inhibition of protein activity using an appropriate assay. Isolated immortalised cells whose culture supernatant contains an antibody that inhibits of the activity of the target protein and has an IC₅₀ of less than 100 ng/ml are then selected and cloned using techniques known to one skilled in the art. The monoclonal antibodies produced by these clones are then isolated according to standard protocols.

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. 81:6851-6855; Neuberger, M. S. et al. (1984) Nature 312:604-608; and Takeda, S. et al. (1985) Nature 314:452-454). Alternatively, techniques described for the production of single chain antibodies can be adapted, using methods known in the art, to produce single chain antibodies specific to the target protein. Antibodies with related specificity, but of distinct idiotypic composition, can be generated by chain shuffling from random combinatorial immunoglobulin libraries (see, for example, Burton D. R. (1991) Proc. Natl. Acad. Sci. USA, 88:10134-10137).

Antibodies can also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. 86: 3833-3837; Winter, G. et al. (1991) Nature 349:293-299).

Antibody fragments which contain specific binding sites for the target protein can also be generated, for example, F(ab′)2 fragments can be produced by pepsin digestion of the antibody molecule and Fab fragments can subsequently be generated by reducing the disulphide bridges of the F(ab′)2 fragments. Alternatively, Fab expression libraries can be constructed to allow rapid and easy identification of monoclonal Fab fragments with the desired specificity (see, for example, Huse, W. D. et al. (1989) Science 246:1275-1281).

Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between the target protein and its specific antibody. Examples of such techniques include ELISAs, radioimmunoassays (RIAs), and fluorescence activated cell sorting (FACS). Alternatively, a two-site, monoclonal-based immunoassay utilising monoclonal antibodies reactive to two non-interfering epitopes, or a competitive binding assay can be used (see, Maddox, D. E. et al. (1983) J. Exp. Med. 158:1211-1216). These and other assays are well known in the art (see, for example, Hampton, R. et al. (1990) Serological Methods: A Laboratory Manual, APS Press, St Paul, Minn., Section IV; Coligan, J. E. et al. (1997, and periodic supplements) Current Protocols in Immunology, Wiley & Sons, New York, N.Y.; Maddox, D. E. et al. (1983) J. Exp. Med. 158:1211-1216).

2.5 Other Compounds

The present invention also provides small molecule inhibitors of the caspase-3 signalling pathway, including peptides, polynucleotides, synthesised organic molecules, naturally occurring organic molecules, vitamin derivatives, carbohydrates, and components or derivatives thereof. The small molecule compounds may bind to proteins of the pathway thereby interfering with the normal activity of the caspase-3 signalling cascade, or they may bind to a nucleic acid encoding wild-type caspase-3, or encoding another protein in the signalling pathway, and thereby interfere with expression of the protein. An example of a small molecule inhibitor of caspase-3 is z-DEVD.fmk (Garcia-Calvo et al. (1998) J. Biol. Chem. 273, 32608-32755). Other effective small molecule inhibitors of caspase-3 include, but are not limited to, nonpeptidic pharmacophores numbered as compounds 3, 4, 47c, 59, 62b, 64b, 66a, 66b, 69b as described by Choong et al. (2002) J. Med. Chem. 45, 5005-5022.

Candidate small molecules to be screened for their ability to act as inhibitors of the caspase-3 signalling pathway can be randomly selected or rationally selected or designed using standard methods known in the art and outlined supra (see Section 1.3).

Selection of Modulators of Caspase-3 Activity

The present invention provides methods for identifying compounds that act as modulators of caspase-3 and affect the cellular fate of stem cells. Screening methods of the invention can use a variety of techniques known in the art to determine the ability of a candidate compound to act as a modulator of caspase-3. For example, the candidate compound may be tested for its effect on the expression of specific proteins in the caspase-3 signalling pathway using the exemplary methods indicated above (see Section 2.1.5). The candidate compound may also be tested for its ability to promote or inhibit the proteolytic activity of caspase-3 or other proteins in the caspase-3 signalling pathway. Alternatively, or in addition, its effect on the caspase-3 signalling pathway can be tested indirectly by determining its ability to affect stem cell differentiation.

Methods of measuring the activity of proteases and kinases are well known in the art and are suitable for use to determine the ability of candidate compounds to activate or inhibit caspase-3, MST1 and other kinases of the caspase-3 signalling pathway (Kameshita, I., & Fujisawa, H. (1989) Anal. Biochem. 183, 139-143; Belizario, J. E., et al., (2001) Br J Cancer 84, 1135-1140; Yaoita, Y., & Nakajima, K. (1997) J. Biol. Chem. 272, 5122-5127; Fernando et al., (2002) Proc. Natl. Acad. Sci. USA 99, 11025-11030). For example, activity may be measured using protein isolation, immunoblot analysis and immunocytochemistry, co-immunoprecipitation and kinase analysis. In addition, kits for measurement of the activity of caspase-3 and other proteins of the signalling pathway are commercially available, for example, the Caspase-3 Fluorometric Assay Kit (BD Pharmingen, San Diego, Calif.), and can also be used to determine the ability of candidate compounds to modulate the activity of caspase-3.

The ability of candidate compounds to affect stem cell differentiation can be determined by culturing stem cells in the presence and absence of the candidate compound and monitoring one or more indicators of differentiation and/or proliferation in the cells. Stem or progenitor cells derived from a variety of tissues can be used to screen the candidate compounds. Examples include, but are not limited to, stem cells from embryonic, cardiac, muscle, pancreatic, neural and liver tissue, stem cells from bone marrow, haematopoietic cells, myoblasts, hepatocytes, thymocytes, cardiomyocytes, and the like.

Methods of maintaining stem cells in culture are known in the art (see, for example, Madlambayan, G. J., et al., (2001) J. Hematother. Stem Cell Res. 10, 481-492; Hierlihy, A. M., et al., (2002) FEBS Lett. 530, 239-243; Asakura, A., et al., (2002) J. Cell Biol. 159, 123-134). The stem cells can be cultured alone (monoculture) or they can be co-cultured with other (educator) cells. As an example, a co-culture could include a population of muscle-derived stem cells (or other stem cells) and myoblasts (educator cells) that are combined after isolation with or without a maintenance phase in separate culture. Alternatively, the two cell populations could be co-cultured as explants (e.g. mouse hindlimb muscle explant) without ever being isolated from their source tissue. It is understood and expected that more than one (monoculture) or two (co-culture) cell populations can be present initially if the stem cell and/or educator cell population is not completely pure, and subsequently when the stem cell population differentiates.

Additional steps may be included in the screening methods before, during, or after the culture period, such as steps to identify or isolate cell populations or otherwise contribute to the success of the assay. For example, growth factors or other compounds may be employed to isolate and expand the stem cell population. EGF and FGF have been used for this purpose with neural stem cells as described by Gritti et al (J. Neurosci. (1999) 19:3287-3297), and Bcl-2 has been used in the isolation of “muscle stem cell” populations (see U.S. Pat. No. 6,337,184).

The stem cells used in the screening assays can be primary cells or cultured stem cell lines isolated or derived from a normal mammal. Alternatively, the stem cells can be isolated or derived from a mammal carrying a mutation in one or more genes encoding a protein in the caspase-3 signaling pathway, for example, from a mammal carrying a caspase-3 mutation that results in reduced caspase-3 activity, such as a null mutation (caspase-3−/−).

Generally, a candidate compound added to the culture is tested over a range of concentrations, typically a 1000-fold range, and a suitable exposure protocol can be readily established by one skilled in the art. When a co-culture is used, stem cell exposure to a candidate modulator can occur before and/or during and/or after the initial exposure of the stem cells to the educator cells. Alternatively, when the candidate modulator is a polynucleotide or a compound encoded by a polynucleotide, such as a protein or polypeptide, the stem cells can be transfected with the polynucleotide, or an expression vector comprising the polynucleotide, using standard methods described herein and elsewhere, such that the candidate modulator is produced endogenously. Additionally, cell lines that stably express a candidate activator or effector can be used in screening assays to determine the effectiveness of the activator/effector in protecting against multiple proliferative stimuli.

Indicators of differentiation and/or proliferation in the stem cells can be monitored qualitatively or quantitatively and include, for example, changes in gross morphology, total cell number, histology, histochemistry or immunohistochemistry, or the presence, absence or relative levels of specific cellular markers. The indicators can be assessed in whole cells or in cellular lysates prepared by standard techniques and can be monitored either with or without the cells or lysates first being subjected to other procedures that would facilitate analysis, such as size fractionation.

The presence, absence or relative levels of various cellular markers can be analyzed using a number of standard techniques. For example, by histochemical techniques, immunological techniques, electrophoresis, Western blot analysis, FACS analysis, flow cytometry and the like. Alternatively, the presence of mRNA expressed from the gene encoding the cellular marker protein can be detected, for example, using PCR techniques, Northern blot analysis, the use of suitable oligonucleotide probes and the like.

Cellular markers that can be assessed as indicators of differentiation are typically lineage-specific proteins. By way of example, cellular markers that may be monitored to assess differentiation of cardiac muscle stem cells include, for example, connexin-43, MEF2C and/or myosin heavy chain. For muscle-derived stem cells, expression of one or more myocyte marker proteins, such as myosin heavy chain, hypophosphorylated MyoD, myogenin, Myf5 and/or troponin T can be measured and for neural stem cells, such as neurospheres or SP cells, the markers GFAP, MAP2 and/or β-III tubulin can be measured (see, for example, Hitoshi, S., et al., (2002) Genes & Dev. 16, 846-858). Examples of cellular markers that may be assessed as indicators of proliferation include, for example, cyclin D1, phospho-histone H1 and H3, E2F and PCNA.

In one embodiment of the present invention, the stem cells employed in the screening method are myoblasts. Induction of differentiation in myoblasts can be measured for example, by measuring myoblast fusion or myotube formation, or by examining the cells for expression of marker proteins such as myosin heavy chain, hypophosphorylated MyoD, myogenin and troponin T. A lack or downregulation of one or more of these markers would indicate that the cells had failed to differentiate. Any increase in proliferation can be assessed by determining total cell numbers and comparing this to a control that was not treated with the candidate compound.

In another embodiment of the present invention, the stem cells used in the screening method are cardiac side-population (SP) cells. Cardiac SP cells have been shown to possess stem cell like activity and a resident population of cardiac SP cells, which can be manipulated experimentally, has been demonstrated to be present in the adult heart [Hierlihy, et al., A. M., et al., (2002) FEBS Lett. 530, 239-243]. Cardiac SP cells can be co-cultured with cardiomyocytes and induction of differentiation can be determined by monitoring the appearance of cardiomyocyte specific markers, such as connexin-43, MEF2C and myosin heavy chain, in the cardiac SP cells. A lack or downregulation of one or more of these markers would indicate that the cells had failed to differentiate.

In another embodiment of the present invention, the stem cells used in the screening method are neural stem cells. Neural stem cells, derived as neurospheres or as side-population (SP) cell fractions can be examined for their innate ability to differentiate into mature neurons when co-cultured with neural cortical progenitor cells. Induction of differentiation can be determined by monitoring the expression of, for example, GFAP, MAP2, β-III tubulin [see, for example, Hitoshi, S., et al., (2002) Genes & Dev. 16, 846-858], myelin basic protein and glial fibrillary acid protein. A lack or downregulation of one or more of these markers, or the presence of the marker nestin, would indicate that the cells had failed to differentiate.

Pharmaceutical Compositions

The present invention provides for pharmaceutical compositions comprising the modulators of caspase-3 protein and a pharmaceutically acceptable diluent or excipient. The pharmaceutical compositions may additionally comprise one or more therapeutic compound, such as an antibiotic, anti-inflammatory, anti-depressant, and the like.

The pharmaceutical compositions of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

When formulated for oral use, the pharmaceutical compositions can be in the form of, for example, tablets, troches, lozenges, aqueous or oily suspensions, dispersible powders or granules, emulsion hard or soft capsules, or syrups or elixirs.

Compositions intended for oral use may be prepared according to methods known to the art for the manufacture of pharmaceutical compositions and such compositions may contain one or more additional agents such as sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents for example, corn starch, or alginic acid; binding agents, for example starch, gelatine or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium, for example peanut oil, liquid paraffin or olive oil.

Aqueous suspensions contain active ingredients in admixture with excipients suitable for the manufacture of aqueous suspensions. Such excipients are suspending agents, for example sodium carboxymethylcellulose, methyl cellulose, hydropropylmethylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethyene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Examples of suitable dispersing or wetting agents and suspending agents include those mentioned above. Additional excipients, for example sweetening, flavouring and colouring agents, may also be present.

Pharmaceutical compositions of the present invention may also be in the form of oil-in-water emulsions. The oil phase may be a vegetable oil, for example olive oil or arachis oil, or a mineral oil, for example liquid paraffin or mixtures of these. Suitable emulsifying agents may be naturally-occurring gums, for example, gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleaginous suspension. This suspension may be formulation according to known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution, lactated Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Administration

The modulators of the present invention and pharmaceutical compositions comprising same may be administered in a number of ways depending upon whether local or systemic treatment is desired and upon the area to be treated. Administration may be topical (including ophthalmic and to mucous membranes including vaginal and rectal delivery), pulmonary, e.g. by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g. intrathecal or intraventricular, administration.

The modulators of the present invention may be delivered in combination with a pharmaceutically acceptable vehicle. Ideally, such a vehicle would enhance the stability and/or delivery properties of the modulator(s). The present invention also provides for administration of the modulators or pharmaceutical compositions using a suitable vehicle, such as a liposome, microparticle or microcapsule. In various embodiments of the invention, the use of such vehicles may be beneficial in achieving sustained release of the active component.

When formulated for parenteral injection, the modulators or pharmaceutical compositions are used in the form of a sterile solution containing other solutes, for example, enough saline or glucose to make the solution isotonic.

For administration by inhalation or insufflation, the modulators or pharmaceutical compositions can be formulated into an aqueous or partially aqueous solution, which can then be utilised in the form of an aerosol. The present invention also contemplates topical use of the modulators or pharmaceutical compositions. For this purpose they can be formulated as dusting powders, creams or lotions in pharmaceutically acceptable vehicles, which are applied to affected portions of the skin.

The dosage requirements for the modulators of the present invention vary with the particular compounds or compositions employed, the route of administration, the severity of the symptoms presented and the particular subject being treated. Dosage requirements can be determined by standard clinical techniques known to a worker skilled in the art. Treatment will generally be initiated with small dosages less than the optimum dose of the compound. Thereafter the dosage is increased until the optimum effect under the circumstances is reached. In general, the modulators are administered at a concentration that will generally afford effective results without causing any harmful or deleterious side effects. Administration can be either as a single unit dose or, if desired, the dosage can be divided into convenient subunits that are administered at suitable times throughout the day.

1. Gene Therapy

The present invention also provides for administration of oligonucleotide, protein, polypeptide and peptide modulators in the form of a genetic vector construct that is designed to direct the in vivo synthesis of the molecule. Suitable vectors include viral vectors, such as an adenoviral, adeno-associated viral, retroviral, lentiviral, baculovirus, or herpes viral vectors. Within the vector construct, the polynucleotide sequence encoding the inhibitor is under the control of a suitable promoter. As described herein, the vector construct may additionally contain other regulatory control elements to provide efficient transcription and/or translation of the polynucleotide encoding the modulator. Such methods of administration are often referred to as “gene therapy.”

Methods of constructing and administering such genetic vector constructs for the in vivo synthesis of oligonucleotides, proteins, polypeptides or peptides are well-known in the art (for example, see Ausubel, et al., (2000) Current Protocols in Molecular Biology, Wiley & Sons, New York, N.Y.; Cid-Arregui (eds.), Viral Vectors: Basic Science and Gene Therapy, (2000) Eaton Publishing Co.).

Thus, for example, cells from a patient may be engineered ex vivo with a polynucleotide (DNA or RNA) encoding one or more modulator (oligonucleotide, protein, polypeptide or peptide), with the engineered cells then being provided to a subject to be treated. Similarly, cells may be engineered in vivo for expression of a polypeptide in vivo by administering to a subject the polynucleotide encoding the modulator. Such methods are well known in the art. The cells can be engineered with the polynucleotide alone or in the form of an expression vector. Typical expression vectors suitable for this use include, for example, expression vectors derived from virus, replication deficient retroviruses, adenovirus, adeno-associated (AAV) virus, herpes virus, or vaccinia virus (see, for example, Cid-Arregui (eds.), Ibid.). Plasmids can also be used, for example, the pVAX1 plasmid from Invitrogen (Carlsbad, Calif.).

As known in the art, a producer cell for producing a viral particle containing RNA encoding a modulator of the invention may be used for administration to a subject in order to engineer cells and express the modulator in vivo. In addition, it is also contemplated that the polynucleotide alone can be administered with or without additional excipients, carrier or delivery molecules. Injection of “naked” DNA is known in the art, for example, see Felgner et al., U.S. Pat. No. 5,580,589.

The present invention also contemplates the use of recombinant DNA constructs to increase expression of an endogenous gene encoding a protein of the caspase-3 signalling pathway. Such constructs and their use to increase expression of a targeted gene are described in U.S. Pat. No. 5,641,670. In general such constructs comprise a regulatory sequence, an exon and a splice donor site, more typically they comprise a targeting sequence, a regulatory sequence, an exon and an unpaired splice-donor site. The construct can be introduced by homologous recombination into the genome of a host cell at a pre-selected site and results in increased expression of a targeted gene. The construct is introduced into the genome in such a manner that a new transcription unit is produced in which the regulatory sequence, the exon and the splice donor site present in the DNA construct are operatively linked to the endogenous gene and the expression of the gene is altered.

2. Transplantation

The present invention also contemplates that the stem cells may be manipulated ex vivo and re-introduced to the patient by transplantation procedures. Such procedures are known in the art.

Uses

The modulators and methods of manipulating stem cell differentiation provided by the present invention have a number of therapeutic applications. The modulators can be used to manipulate the fate of endogenous stem cells in vivo or they can be used in vitro or ex vivo to provide expanded or differentiated stem cell populations for transplantation. Modulators can be selected according to the desired end result for a stem cell population. Thus, for example, if a differentiated stem cell population is required then a modulator that activates caspase-3 can be selected and applied to the stem cells. On the other hand, if an expanded stem cell population is required, a modulator that inhibits caspase-3 can be selected. The present invention also contemplates the use of combinations of the modulators, for example, use of one or more inhibitor of caspase-3 to first expand a stem cell population followed by the use of one or more activator to stimulate differentiation once the desired number of cells has been achieved.

For in vivo applications, for example, the modulators can be administered to a subject in order to stimulate resident stem cells to proliferate and/or differentiate and thereby replace or repair damaged or defective tissue. Thus, the modulators and methods of the present invention would be useful to replace damaged tissue, for example, after chemotherapy or radiation therapy, in the treatment or management of diseases and disorders which result in degeneration or damage of tissue, such as muscular dystrophy, cardiovascular disease, stroke, heart failure, myocardial infarction, neurodegenerative diseases, such as Parkinson's disease and Alzheimer's disease, and the like, or in the treatment or management of diseases and disorders in which there is a need to replace lost, damaged or malfunctioning tissue, such as cancers (including leukemias), degenerative liver diseases, including cirrhosis and hepatitis, diabetes, and degenerative or ischemic cardiac disease.

The modulators and methods can also be used to stimulate stem or progenitor cell proliferation and/or differentiation in vitro in order to provide replacement tissue for transplantation. Ex vivo expansion of stem cells has obvious therapeutic indications for treating numerous disease conditions. For example, bone marrow transplantation is a well documented therapy for treating blood borne cancers such as leukemias, and ex vivo expansion of haematopoietic stem cells (HSCs) and umbilical cord blood (CB) cells are a recognised extension of traditional haematotherapy (Douay (2001) J. Hematother Stem Cell Res. 10, 341-346).

The present invention also contemplates the use of the modulators and methods of the present invention to promote proliferation and/or differentiation of stem cells in vitro where the population of cells is destined for further in vitro use, for example for research purposes. The modulators and methods also have potential applications in tissue engineering, including the development of new in vitro models for drug testing.

Kits

The present invention additionally provides for therapeutic kits containing one or more modulator of caspase-3 for the modulation of stem cell differentiation. The modulator(s) may be provided in the form of one or more pharmaceutical composition. The contents of the kit can be lyophilised and the kit can additionally contain a suitable solvent for reconstitution of the lyophilised components. Individual components of the kit can be packaged in separate containers and, associated with such containers, can be a notice in the form prescribed by a governmental agency regulating the manufacture, use or sale of pharmaceuticals or biological products, which notice reflects approval by the agency of manufacture, use or sale for animal administration. The kit can further comprise other therapeutic compounds which can be employed in conjunction with the modulators of the present invention.

To gain a better understanding of the invention described herein, the following examples are set forth. It should be understood that these examples are for illustrative purposes only. Therefore, they should not limit the scope of this invention in any way.

### EXAMPLES

### Example 1

### Modulating Differentiation in Muscle Stem Cells (Myoblasts)

General: Materials and Methods

Myoblast culture. C2C12 cell (mouse skeletal muscle myoblast) cultures were grown and maintained in DMEM containing 10% FBS with 50 units/ml penicillin and 50 μg/ml streptomycin (Gibco). Differentiation was induced by incubating cultures in DMEM containing only a low serum concentration (2% horse serum). Caspase activity was measured using a Caspase-3 and Caspase-8 Fluorometric Assay Kit (Pharmingen). Cells in which caspase activity was perturbed were treated with 20 μM of inhibitors specific for either caspase-3 (z-DEVD.fmk), or caspase-8 (z-LETD.fmk) (Enzyme Systems) (based on concentration curve tests of 0 to 100 μM). Control cells were treated with an equivalent amount of vehicle (DMSO). Primary myoblast cells were derived from skeletal muscles of the hind- and fore-limbs from 1-2 day old C57BL/6 wild-type, caspase-3 heterozygous and caspase-3 null mice (Frasch, S. C., et al., (1998) J. Biol. Chem. 273, 8389-8397; Kuida, K., et al., (1996) Nature 384, 368-372) (n=5 for each genotype) as described (Cregan, S. P. et al., (1999) J. Neuro. 19, 7860-7869.). To induce differentiation, primary myoblast cultures were incubated in DMEM containing 5% horse serum.

Transfection Assays. The complete open reading frame of the human MST1, [Genbank Accession No. gi|1117790] obtained from a clone bank collection (Stratagene) was inserted into the expression vector pcDNA3.1 Myc/His (Invitrogen). A constitutively active MST1 (MST1-act) was prepared as previously described (Megeney, L. A., et al., (1996) Genes Dev 10, 1173-1183). The integrity of both MST1 and MST1-act were confirmed by sequencing. The kinase activity of these protein products were verified by transient transfection into COS1 cells followed by immunoprecipitation and kinase analysis using Histone-H1 as the target substrate (see below). Transfections with MST1 and MST1-act into both COS1 and primary myoblasts were performed using Lipofectamine Plus (Gibco) as directed in the manufacturer's instructions.

Protein isolation, immunoblot analyses and immunocytochemistry. Cells from C2C12 and primary myoblast cultures were washed with ice-cold PBS and lysed on ice for 45 minutes in modified-RIPA buffer containing 20 mM NaF, 5 mM Na₃VO₄ and 10 μg/ml each of aprotinin, leupeptin, pepstatin and PMSF. Total protein was measured using the BCA protein assay (Pierce). Immunoblot analyses were performed as described (Creasy, C. L., & Chernoff, J. (1995) J. Biol. Chem. 270, 21695-21700) using antibodies for MST1, MKK6, p38γ, and p38α (Upstate), p38 and MEF2C (Cell Signaling Technologies), myogenin F5D and myosin heavy chain MF20 mAb (Developmental Hybridoma Bank), and active caspase-3 (Biovision).

C2C12 myoblasts were fixed and stained with anti-MF20 as previously described (Cregan, et al., (1999) J. Neuro. 19, 7860-7869). Primary myoblast cultures were fixed in 4% paraformaldehyde, rinsed three times in PBS containing 0.01% Triton X-100 and stained as above. For both C2C12 and primary myoblast cultures, nuclei were detected by incubating cells for 10 min with 4,6-diamidino-2-phenylindole diluted 1:10000 in PBS.

Co-immunoprecipitation and kinase analyses. Immunoprecipitation was performed using 150-200 μg of total protein, incubated in modified-RIPA buffer at 4° C. for 16 h containing the respective antibody. Samples were mixed for 1 h with 25 μl of a 50% Protein G (Pharmacia). For co-immunoprecipitation, immunoprecipitates were washed in modified-RIPA buffer, boiled in 1× Lamelli buffer, separated by SDS-PAGE and Western blotted for immunoblot analyses. Immunoprecipitates used for kinase analyses were washed in modified-RIPA buffer and incubated in kinase buffer containing 20 mM MOPS (pH 7.2), 25 mM β-glycerophosphate, 5 mM EGTA, 1 mM Na₃VO₄ and 1 mM DTT supplemented with [γ-³²P]ATP and either recombinant MBP or recombinant Histone H1 (Upstate) as the target substrate. Samples were incubated for 30 min at 37° C. and then boiled in 1× Lamelli buffer. Following SDS-PAGE, gels were dried and phospho-incorporation was analyzed by autoradiography.

Samples were subjected to in-gel kinase analyses as previously described (Kolodziejczyk, S. M., et al., (1999) Curr Biol 9, 1203-1206) using MBP or Histone H1 as the target substrate. For two-dimension in-gel kinase analyses, samples were isoelectric focused in the first dimension using pH 4.0-7.0 IPG strips (BioRad). Second dimension analyses was performed using 10% SDS-PAGE containing Histone H1 as the in-gel substrate. Additional in-gel kinase analyses was performed as above.

In vitro translation and MST1 proteolysis. MST1-pcDNA3.1 Myc/His was in vitro transcribed and translated using a TnT T7 Coupled Wheat Germ Extract System (Promega). The labelling efficiency was verified by SDS-PAGE, Coomassie-staining and autoradiography. MST1 protein labelled with [³⁵S]-methionine was incubated for 30 min at 37° C. with samples of C2C12 cells following z-DEVD.fmk (caspase-3 inhibitor) and low-serum media treatment. The proteolytic cleavage of MST1 was assessed by SDS-PAGE and autoradiography.

1.1 Caspase-3_{-/-} Myoblasts Display a Differentiation Deficit

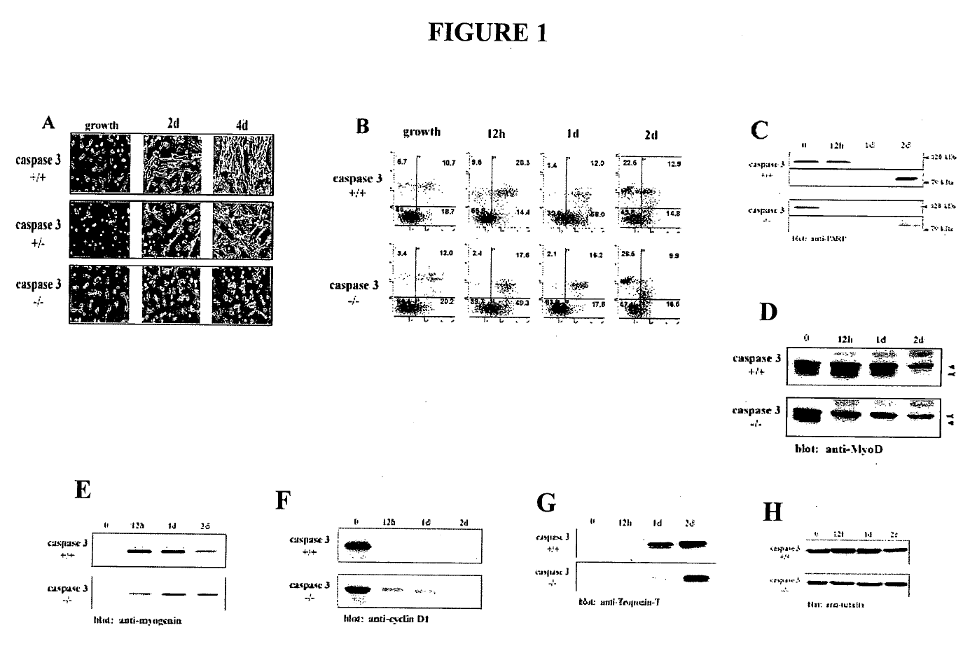

Mice carrying a targeted null mutation in caspase-3 were raised and, as previously reported, those which survived to early perinatal life were strikingly smaller relative to their wildtype and heterozygote littermates (Frasch, S. C., et al., (1998) J. Biol. Chem. 273, 8389-8397; Kuida, K., et al., (1996) Nature 384, 368-372). Furthermore, a visible reduction in total skeletal muscle mass was observed indicating that myogenesis may be compromised in these animals. To examine the effect of caspase-3 ablation on myogenesis, primary myoblast cultures were generated from 1-2 day old mice carrying the caspase-3 null mutation (caspase-3 −/−). Visual examination of cell cultures showed that the proliferation of wildtype, caspase-3_{+/-} and caspase-3_{-/-} myoblasts were similar. Following induction of differentiation on day 0, a severe lack of myotube formation was evident by post-induction day 4 in caspase-3_{-/-} cultures relative to wildtype cells (FIG. 1A; compare a fusion index of 10% vs. 80%, null to wildtype, respectively). In addition, the expression of a number of differentiation specific proteins was substantially reduced in caspase-3_{-/-} myoblasts relative to wildtype myoblasts, including hypophosphorylated MyoD, myogenin and troponin T (FIG. 1D, E, H respectively). Furthermore, in caspase −/− myoblasts, the levels of cyclin D1, a marker of cellular proliferation remained elevated compared to wildtype myoblasts (FIG. 1F). These observations suggest that elevated caspase-3 activity is necessary for effective differentiation during the early stages of skeletal myogenesis.

Conceivably, caspase-3 activity may be required to remove a population of myoblasts (through apoptosis) which would normally inhibit the differentiation process, i.e., a non-cell autonomous effect. Alternatively, elevated caspase-3 activity may lead to intracellular alterations, which in turn activate a differentiation program i.e., a cell autonomous effect. Therefore, the degree of apoptosis in caspase null and wildtype myoblasts was compared during low serum induction of differentiation. Surprisingly, there was no measurable difference in early stage or late stage markers of apoptosis as demonstrated by flow cytometry using Annexin V and propidium iodide staining and analysis of poly(ADP-ribose) polymerase (PARP) cleavage (FIG. 1B, C—Both the 118 kDa PARP protein and the 85 kDa cleavage fragment were detected). Furthermore, myocytes from caspase-3 heterozygote mice displayed a reduced level of myogenesis relative to wildtype controls but differentiated nonetheless (FIG. 1A). These results suggest the elevated caspase-3 activity normally associated with differentiation is not strictly apoptotic.

In more detail, FIG. 1 shows: (A) Primary myoblast cultures derived from wildtype and caspase-3_{-/-} mice (n=5 for each genotype) fixed and stained for myosin heavy chain after incubation in low serum media for 2 or 4 days. Cells were counterstained with the nuclear marker hematoxillin (dark staining pattern). A severe lack of myogenesis is noted in caspase-3 −/− cells. (B) Flow cytometry analysis of wildtype and caspase-3 −/− myoblasts after incubation in low serum medium for the indicated period. Cells were either left unstained (lower left quadrant), stained with Annexin V-FITC (lower right quadrant), propidium iodide (upper left quadrant) or both (upper right quadrant). A comparable proportion of apoptotic cells was observed for both wildtype and caspase-3 null myoblasts after 2 days of low serum treatment. (C) Poly(ADP-ribose) polymerase (PARP) is equivalent in for both wildtype and caspase-3 null myoblasts. Western blot analysis of PARP using an anti-PARP antibody in wildtype and caspase-3 null myoblasts incubated in low serum (differentiation) media. Both the 118 kDa PARP protein and the 85 kDa cleavage fragment were detected. A reduction of muscle differentiation specific proteins in caspase-3 null myoblasts was observed using Western blot analyses for hypophosphorylated MyoD (D) (top and bottom arrows on each panel indicate the position of phosphorylated and hypo-phosphorylated MyoD respectively), myogenin (E) and troponin T (H). (F) Caspase-3 null myobasts displayed a prolonged accumulation of cyclin D1 in the presence of low serum (differentiation) media. (G) Equal loading for the Western blot analyses was assessed using an anti-tubulin antibody.

1.2 Caspase-3 Activity has a Positive Role During Skeletal Muscle Differentiation

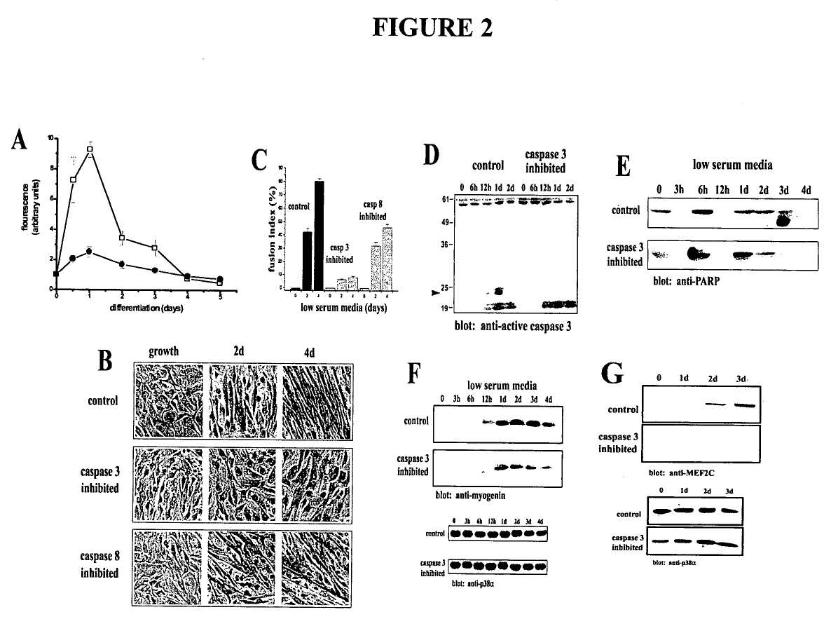

The phenomena observed in the myoblasts derived from caspase-3 null animals was further examined in an established myoblast cell population. As previous studies have reported changes in the activities of caspases-3 and -8 in skeletal muscle (Kameshita, I., & Fujisawa, H. (1989) Anal. Biochem. 183, 139-143; Belizario, J. E., et al., (2001) Br J Cancer 84, 1135-1140; Yaoita, Y., & Nakajima, K. (1997) J. Biol. Chem. 272, 5122-5127), the activities of caspases-3 and -8 in the C2C12 skeletal muscle cell line were measured during onset and progression of differentiation. Proliferating C2C12 cells were placed in low serum media and allowed to differentiate for five days. At various time points over the 5 days, subsets of cells were washed extensively to ensure that lysates were free of non-adherent apoptotic cells. Fluorometric analyses revealed a sharp, nine-fold increase in caspase-3 activity within 24 h, which then declined as differentiation progressed (FIG. 2A). Interestingly, although a similar trend in activity was noted for caspase-8, the increase in activity relative to normal values was not as large as that observed with caspase-3 (mean±SEM, n=5). Immunocytochemical detection of active caspase-3 confirmed that the measured increase in caspase-3 activity was a feature of differentiating myoblasts and not a result of cells undergoing apoptosis.

As a compliment to the analyses of the caspase-3 null model, caspase activity in C2C12 myoblasts was directly targeted using pharmacologic inhibitors specific for caspase-3 (z-DEVD.fmk) and 8 (z-LETD.fmk). Cells were fixed after 0, 2 and 4 days of differentiation and stained with the myosin heavy chain antibody MF20. A minor attenuation of myotube formation is observed with caspase-8 inhibition. Caspase-3 inhibited myoblasts displayed an impaired formation of myotubes (FIG. 2B middle panel, C). Specifically, these cells remained mononucleated and unfused after 4 days of low serum conditions relative to vehicle (DMSO) treated C2C12 myoblasts. Western analyses with a caspase-3 specific antibody on peptide treated and control cell lysates confirmed that z-DEVD.fmk treatment was effective in inhibiting caspase-3 activity (FIG. 2D—The arrow indicates a 21 kDa caspase-3 fragment, indicative of caspase-3 activation, which is abundant in control cells and at very low levels in caspase-3 inhibited samples). Of note, the lack of myoblast differentiation with z-DEVD.fmk treated C2C12 cells was remarkably similar to caspase-3^{-/-} myoblasts. In contrast, differentiation was more evident in cells that were incubated with caspase-8 inhibitor, z-LETD.fmk (FIG. 2B bottom panel, C). However, the degree of differentiation remained below that observed in vehicle treated cells. Although a proportion of caspase-3 inhibited cells were able to express the muscle transcription factor myogenin (FIG. 2F), a delayed and attenuated accumulation was evident when compared to control myoblasts. More strikingly, the expression of the late myogenic marker MEF2C was virtually absent in cells inhibited for caspase-3 activity (FIG. 2G). Clearly, the deficit in myogenesis brought about by caspase-3 inhibition suggests that caspase-3 is a pro-myogenic protease and that its effects are greater than that mediated by caspase-8. In addition, PARP cleavage products accumulated to a similar degree in z-DEVD treated and untreated C2C12 myoblasts (FIG. 2E), suggesting that caspase-3 activity is not responsible for the ambient rate of apoptosis for differentiating myoblast populations.

In more detail, FIG. 2 shows: (A) fluorometric analysis: a sharp increase in caspase-3 activity (squares) was found in C2C12 myoblasts after 1 day of low serum treatment. A much smaller increase in caspase-8 activity (circles) was noted although a similar trend in activation was found (mean±SEM, n=5). (B) Cellular morphology of C2C12 cells after inhibition of caspase-3 or caspase-8 activity. C2C12 cells were incubated in low serum media in the presence of either a caspase-3 specific inhibitor (Z-DEVD.fmk), a caspase-8 specific inhibitor (Z-LETD.fmk) or control (DMSO). Cells were fixed after 0, 2 and 4 days of differentiation and stained with myosin heavy chain antibody MF20. Myotube formation is drastically attenuated in caspase-3 inhibited cells after 4 d of differentiation. A minor attenuation of myotube formation is observed with caspase-8 inhibition. (C) Caspase-3 inhibited cells lack fusion capacity. Calculation of myoblast fusion indices as a percentage of cells containing two or more nuclei within a differentiated myotube (MF20 positive cell). Values were determined as mean±standard error from 34 independently derived primary cultures. (D) Western blot analysis of active caspase-3 from control and caspase-3 inhibited cells. The arrow indicates a 21 kDa caspase-3 fragment, indicative of caspase-3 activation abundant in control cells and at very low levels in caspase-3 inhibited samples. (E) Comparable levels of PARP cleavage in both control and caspase-3 inhibited cells. PARP cleavage in control and caspase-3 inhibited cells assessed by Western blot. Shown is the 118 kDa PARP species. (F) Accumulation of myogenin is delayed and is in lower abundance in caspase-3 inhibited C2C12 myoblasts and control cells. Equal loading was assessed by Western blot using anti-p38α. (G) Caspase-3 inhibited C2C12 myoblasts express low levels of MEF2C relative to control cells. Expression of MEF2C in caspase-3 inhibited and control cells. Equal loading was assessed as in (F).

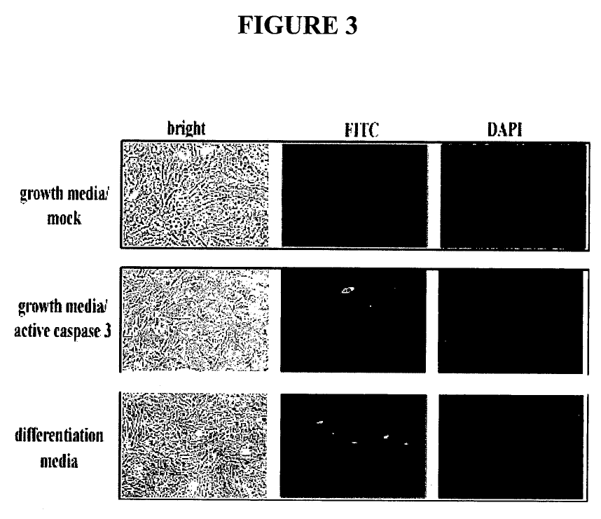

To further test a cell-autonomous role of caspase-3 in inducing myoblast differentiation, active caspase-3 protein was transfected into a subconfluent population of C2C12 myoblasts maintained in growth media. Sub-confluent C2C12 myoblasts were transfected with 20 pg of recombinant active caspase-3 and examined after 12 h for the presence of myosin heavy chain (anti-MF20) detected using a FITC conjugated secondary antibody. A confluent population of cells were incubated in low serum media (differentiation media) to induce myoblast differentiation and examined after 12 h. In all cells examined, nuclei were localised with DAPI staining. The results are shown in FIG. 3, which is representative of 7 trials.

Transfected myoblasts initiated the differentiation program, as measured by immunodetection of myosin heavy chain and morphologic reorganisation (FIG. 3; transfection efficiency ˜10%; transfected cells undergoing differentiation >95%, n=4 independent transfections). Myoblasts expressing myosin heavy chain were also positive for the transfected active caspase-3 protein (not shown) and did not display any morphologic features normally attributed to apoptosis. Moreover, the transfection of active caspase-3 was permissive to inducing myoblast differentiation irrespective of growth stimulating conditions, i.e. high serum conditions (FIG. 3). Taken together, these observations indicate that endogenous caspase-3 activity is required for myoblast differentiation.

1.3 Caspase-3 Activates MST1 Kinase During Differentiation

When processed to an active form, caspase-3 is capable of targeting several protein kinases rendering them active, usually through cleavage and removal of their regulatory C-terminal domain (Utz, P. J., & Anderson, P. (2000) Cell Death Differ. 7, 589-602; Mukasa, T., et al., (1999) Biochem. Biophys. Res. Commun. 260, 139-142). Therefore, caspase-3 activation may be necessary to provoke a kinase(s) that is part of the myogenic Signaling cascade. Indeed, various promyogenic kinases from the MAPK family (Cheng, T. C., at el., (1993) Science 261, 215-218; Cuenda, A., & Cohen, P., (1999) J. Biol. Chem. 274, 4341-4346; Yang, S. H., et al., (1999) Mol. Cell. Biol. 19, 40284038) have been shown to be cleavage-activated by caspase-3 or associated with upstream kinase effectors that are themselves stimulated through a caspase directed cleavage event (Utz, P. J., & Anderson, P. (2000) Cell Death Differ. 7, 589-602; Mukasa, T., et al., (1999) Biochem. Biophys. Res. Commun. 260, 139-142).

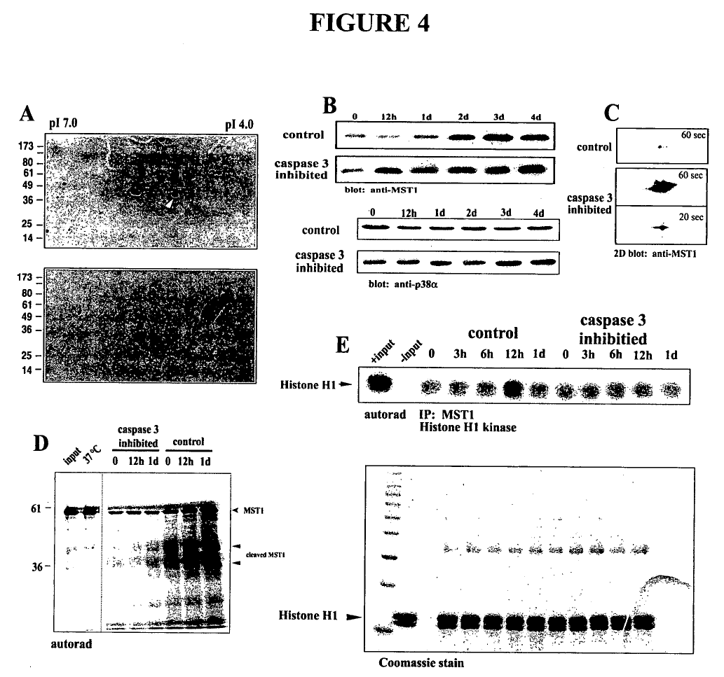

A two-dimensional in-gel kinase assay was employed using myelin basic protein (MBP) as a target substrate to identify protein kinases that may be targeted by caspase-3 activity. C2C12 cells were treated with caspase-3 inhibitor and total protein lysates were isoelectrically focused in the first dimension and then separated in the second dimension using MBP as the crosslinked substrate. Following a 12 h low serum treatment of C2C12 myoblasts with z-DEVD.fmk, a number of endogenous kinases had a diminished level of activity when compared to untreated myoblasts (FIG. 4A). Similar results were seen using histone H1 as the in-gel substrate.

One kinase that was altered in activity co-migrated with a predicted pI (5.2) and molecular weight (36 kDa) consistent with Mammalian Sterile Twenty-like kinase (MST1). MST1 is a known caspase-3 activated kinase homologous to the yeast family of serine/threonine Ste20 kinases (Lechner, C., et al., (1996) Proc. Natl. Acad. Sci. USA 93, 43554359; Megeney, L. A., et al., (1996) Genes Dev 10, 1173-1183). Interestingly, MST1 has been described to act as an upstream kinase effector targeting MKK6 and members of the p38 MAPK family (Lechner, C., et al., (1996) Proc. Natl. Acad. Sci. USA 93, 4355-4359 Sun, S., & Ravid, K. (1999) J. Cell. Biochem. 76, 44-60). Analyses by Western blot revealed a lower level of full-length MST1 during the initial 24 h of low serum treatment compared to z-DEVD.fmk treated cultures (FIG. 4B). In contrast, MST1 protein remained relatively unchanged in z-DEVD.fmk treated cultures. This difference was further exemplified using two-dimensional immunoblot analyses (FIG. 4C). To verify that the difference in MST1 accumulation was due to caspase-3 mediated cleavage, ³⁵S-methionine labelled MST1 was incubated with protein lysates isolated from both control and z-DEVD.fmk treated C2C12 cultures. The full length MST1, migrated to approximately 58 kDa on 10% SDS-PAGE. When incubated with lysates from normally differentiating C2C12 cells, a lower band of approximately 36 kDa appeared, indicative of a cleaved MST1 fragment (FIG. 4D). An additional band of approximately 45 kDa was also present in these samples. Recent reports have identified two sites within the MST1 amino acid sequence that are susceptible to caspase cleavage (Sun, S., & Ravid, K. (1999) J. Cell. Biochem. 76, 44-60). One of these sites between amino acids 323-327, is the conserved caspase-3 cleavage motif DEVD. Cleavage at this site generates a 36-kDa catalytically active fragment (Lechner, C., et al., (1996) Proc. Natl. Acad. Sci. USA 93, 4355-4359). Cleavage at a second site (TMTD) between amino acids 346-349 generates a 41 kDa fragment, which may represent a functional caspase-8 cleavage domain. In C2C12 cells, the lower 36 kDa MST1 fragment was not apparent in samples where caspase-3 activity was inhibited suggesting that MST1 is normally cleaved by caspase-3 during the early phase of myogenesis. Given that the targeted cleavage of MST1 is tightly coupled to its activation, the activity of MST1 during early periods of differentiation was examined. MST1 was immunoprecipitated from control and z-DEVD.fmk treated lysates and incubated with Histone H1 in an in vitro kinase assay. Histone H1 was strongly phosphorylated in control lysates after 12 h in low serum media (>4-fold increase), whereas no phosphorylation above basal levels was detectable from z-DEVD.fmk treated cells (FIG. 4D). Together, these results support a mechanism whereby caspase-3 mediates the activation of MST1.

In more detail, FIG. 4 shows: (A) Two-dimensional in-gel kinase assay. C2C12 cells were treated with caspase-3 inhibitor and total protein lysates were isoelectric focused in the first dimension and then separated in the second dimension using MBP as the cross-linked substrate. Caspase-3 inhibition results in a significant loss of MBP-directed cellular kinase activities. The putative location of active MST1 kinase, based on predicted pI (5.2) and molecular weight (36 kDa), is indicated by the white arrowhead. (B) Western blot analysis of C2C12 cells using an anti-MST1 antibody. Control treated cells show a varied amount of MST1 during 0 and 1 day of differentiation and exemplify a proteolytic-associated event. MST1 does not change upon the addition of low serum media in caspase-3 inhibited myoblasts. Shown below is loading control using p38α. (C) 2D IEF/Western blot of control and caspase-3 treated cells after 12 h in low serum media. Subsequent immunoblot analysis was performed using an anti-MST1 antibody. Shown are 1 min exposures for both control and caspase-3 inhibited samples. A 20 s exposure for the caspase-3 sample is included as a comparison of signal intensity. (D) MST1 is cleaved in differentiating myoblasts. [³⁵S]-methionine labelled MST1 was incubated with lysates from either control or caspase-3 inhibited C2C12 cells under differentiation conditions. The arrows indicate MST1 cleavage products of approximately 36 and 45 kDa that are present in control samples but absent in caspase-3 inhibited samples. (E) MST1 kinase activity increases during myoblast differentiation. γ-³²P-[ATP] labelled Histone H1 following immunoprecipitation of MST1 from C2C12 myoblasts under differentiation conditions. A substantial activation of MST1 in the control sample after 12 h of differentiation coincides with the cleavage of MST1 as shown in (A) and (C). Sample loading was indexed by staining the gel with Coomassie blue prior to drying and autoradiography (bottom panel of D). The arrow indicates the position of histone H1.

Earlier reports have implicated members of the p38 MAPK pathway as substrates for MST1 (Lechner, C., et al., (1996) Proc. Natl. Acad. Sci. USA 93, 4355-4359; Sun, S., & Ravid, K. (1999) J. Cell. Biochem. 76, 44-60). These kinases have been previously demonstrated to promote myogenesis by phosphorylating and increasing the endogenous activity of skeletal muscle transcription factors. The potential of these kinases as downstream substrates in a caspase-3/MST1 signal conduit, therefore, was examined. Of interest, caspase-3 inhibition led to a block in the activation for both MKK6 and p38γ. Taken together, these data indicate that MST1 serves to enhance downstream members of the MAPK cascade which effectively promote skeletal muscle differentiation.

1.4 Active MST1 Rescues Myogenesis in Caspase-3^{-/-} Myoblasts

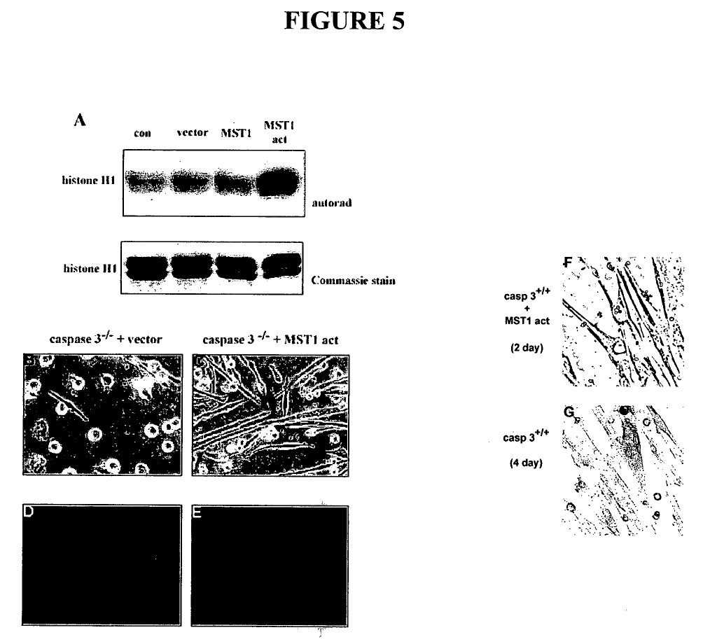

To delineate the effect of MST1 on myogenesis, wild-type MST1, an activated MST1 (aa 1-330, MST1-act) or plasmid alone was transiently transfected into primary myoblasts derived from caspase-3^{-/-} mice. The activity of these constructs was verified by Histone H1 targeted kinase analysis of MST1 from transiently transfected COS1 cells (FIG. 5A). MST1-act transfected cells display a high level of kinase activity. Caspase-3^{-/-} myoblasts transfected with either plasmid alone or wild-type MST1 remained as mono-nucleated myoblasts (FIG. 5B, D). In contrast, caspase-3^{-/-} myoblasts transfected with MST1-act had elongated and fused with neighbouring cells to form multinucleated myotubes (FIG. 5C, E) indicating that expression of activated MST1 in caspase-3^{-/-} myoblasts rescued the aberrant myogenic phenotype. To further demonstrate the pro-myogenic role of activated MST1, MST1-act was transfected into wildtype primary myoblasts. Transfected wildtype cultures left in differentiation media for 2 days had phenotypic characteristics that were comparable to wildtype cultures after 4-5 days of differentiation (FIG. 5F, G). Furthermore, MST1-act transfected cells subject to a protracted differentiation time course (>4 days) were observed to display enhanced apoptosis. These results indicate that the endogenous activation pattern of MST 1 accelerates skeletal myocyte differentiation, although potent apoptotic events can be engaged by this kinase during extended periods of activation.

In more detail, FIG. 5 shows: (A) Kinase activity of MST1 protein constructs. Transfection of full-length MST1 or truncated MST1 (aa 1-330, MST1-act) into COS1 cells (n=4). Following transfection, MST1 was immunoprecipitated and used in a Histone H1 targeted kinase assay. Accuracy of sample loading is indicated by Coomassie staining. (B) Caspase-3 knockout myoblasts transfected with vector alone failed to differentiate. (C) Caspase-3 knockout myoblasts containing MST1-act differentiated to form fused, multinucleated myotubes. The efficiency of differentiation in MST1-act myoblasts was >85%. Total nuclei were visualized using DAPI staining (D and E). Nuclei are more numerous and more closely associated with myoblasts containing MST-act (panel E) than vector alone (panel D). Wildtype myoblasts transfected with MST1-act (F) demonstrated an accelerated myogenesis after 2 days relative to untransfected wildtype myoblasts and were comparable to untransfected wildtype myoblasts left to differentiate for 4 days (G).

The above examples have demonstrated that both biologic and chemical blockade of caspase-3 results in a profound deficiency in myogenesis and also that the Ste20-like kinase MST1 serves as a fundamental conduit in mediating caspase-3 induction of muscle differentiation. Previous reports have suggested that caspase-3 is capable of regulating non-apoptotic functions in certain cell types, i.e. nuclear extrusion in differentiating lens epithelium and keratinocytes (Graves, J. D., et al., (2001) J. Biol. Chem. 276, 14909-14915; Ishizaki, Y, et al., (1998) J. Cell. Biol. 140, 153-158) and T-cell activation (Weil, M., et al., (1999) Curr. Biol. 9, 361-364). Nevertheless, this is the first observation to directly link caspase-3 activity to such a profound alteration in cell morphology without ushering in a death like phenotype. It is important to note that similar signalling components (MST1, MKK6, p38) are also found in numerous differentiated cell types including hepatocytes, thymocytes and neurons (Alam, A., et al., (1999) J Exp Med. 190,1879-1890; Zheng, T. S., et al., (1998) Proc. Natl. Acad. Sci. USA. 95, 13618-13623; Ko, H. W., et al., (2000) J. Neurochem. 74, 2455-2461) and thus caspase-3/MST1 signalling may be an indispensable component for initiating cellular differentiation in general.

### Example 2

### Localization of Caspase-3 and Pro-Caspase-3 in Various Stem Cell Populations

In order to explore the possibility that pro-caspase 3 (i.e. in its uncleaved, inactive form) is behaving as a scaffold protein, pro-caspase 3 and active-caspase 3 localization studies were conducted. The term “scaffold protein” indicates that pro-caspase 3 forms molecular complexes with other signaling proteins that are required for inducing a differentiation effect (or an apoptotic effect) in stem cells. The advantage of behaving as a scaffold protein is that given the correct stimulus, i.e. a stimulus to differentiate rather than induce cell death, the pro-caspase 3 form can be quickly activated and present itself to key signal pathways that promote a pro-differentiation effect. As with most signal transduction cascades, the response of caspase-3 is likely dose- and time-dependent. Thus, by acting as a scaffold protein, caspase-3 has a better chance of producing the correct effects that the cellular environment demands (i.e. proliferation or differentiation).

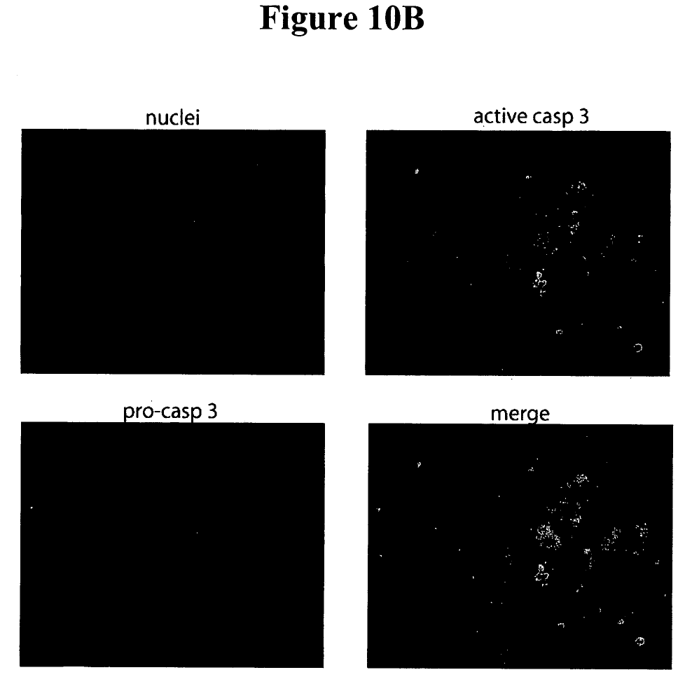

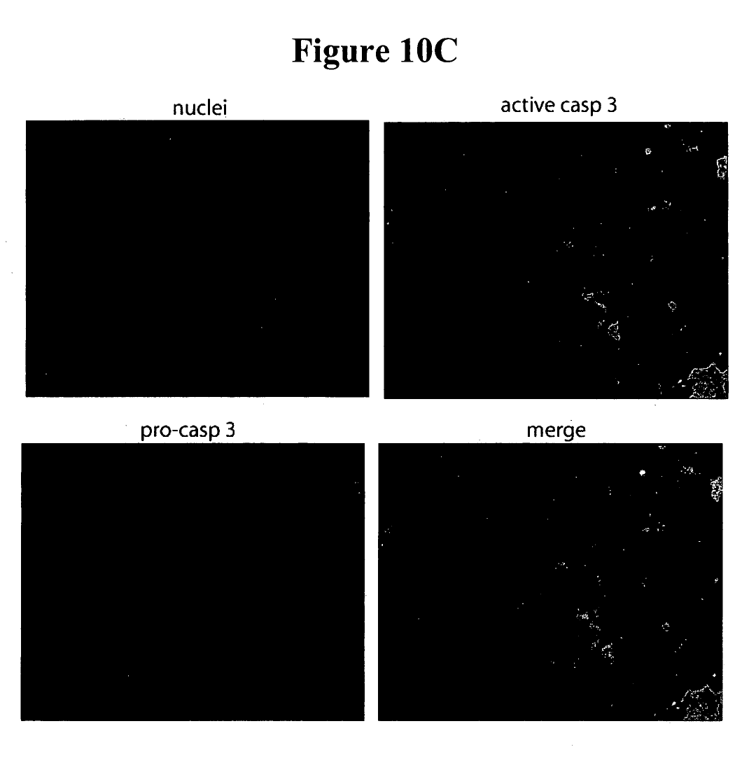

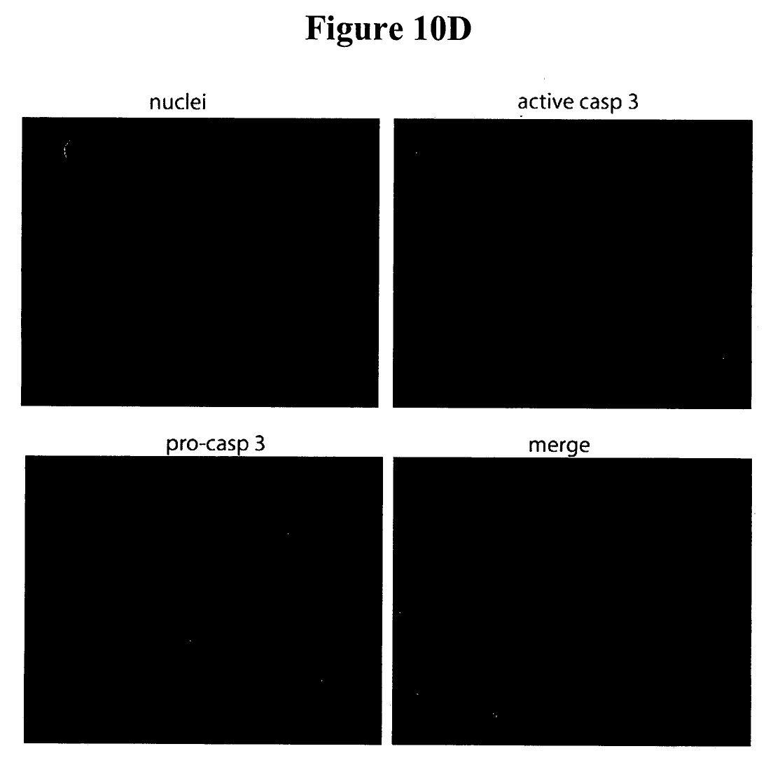

FIG. 10 shows the localization of pro-caspase 3 and active caspase 3 in proliferating and differentiating myoblasts. (A) shows staining for pro-caspase 3 in (Cy3) red and the cleaved form, active caspase 3 in FITC (green) during skeletal myoblast growth (proliferation). Pro-caspase 3 is distributed throughout the cell. Active-caspase 3 is also distributed throughout the cell with punctuate accumulation around the nucleus (shown in blue with DAPI stain). The merge of both pro- and active-caspase 3 shows the co-localization of both proteins. (B) A redistribution of pro- and active-caspase 3 is observed during skeletal myoblast differentiation. Active-caspase 3 shown by FITC (green) remains localized throughout the cell, however, a more punctuate pattern of accumulation is observed around the nucleus (shown in blue with DAPI stain). Pro-caspase 3 has less punctuate accumulation around the nucleus (relative to growth cells) and shows a more dispersed pattern throughout the cell. (C) and (D) Active-caspase 3 disappears during later stages of myoblast differentiation whereas pro-caspase 3 remains distributed throughout the cell.

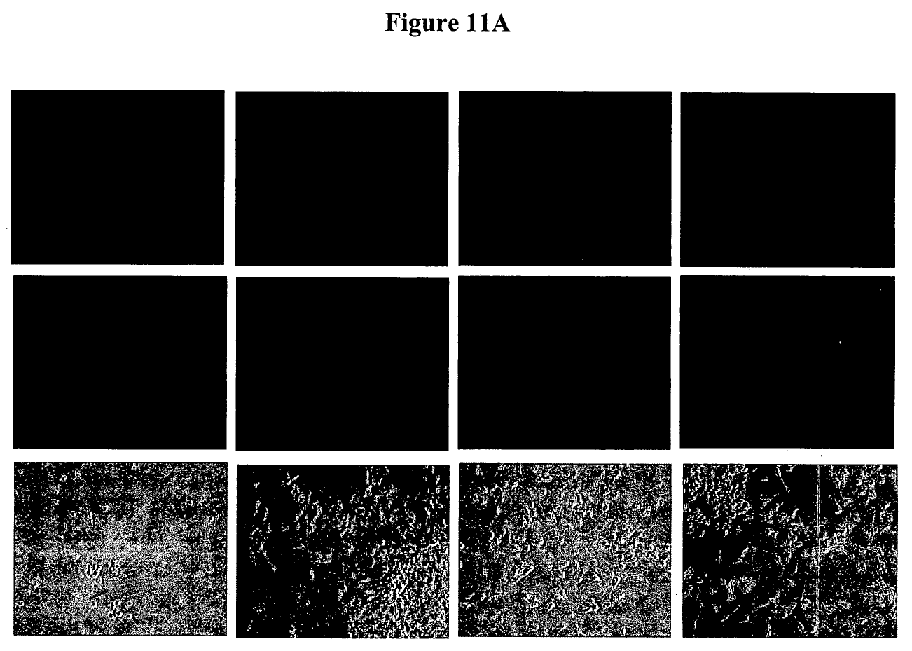

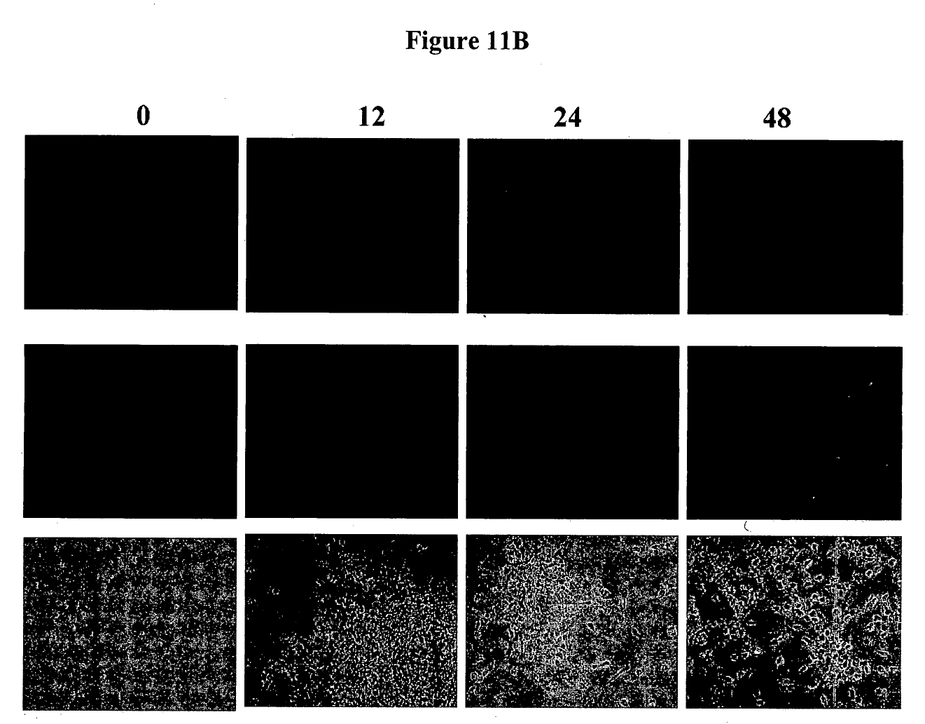

FIG. 11 shows the localization of pro-caspase 3(A) and active caspase 3 (B) in proliferating and differentiating primary striatal stem cells. The figure indicates that cells detaching from the main group of cells (neurosphere) show positive staining for active caspase 3 whereas cells not yet detached (i.e. likely not differentiated) stain positive for pro-caspase 3.

For methodology relating to the localization and visualization of caspase-3 and pro-caspase-3 in myoblasts and striatal stem cells, see Example 3 (below).

### Example 3

### Inhibition of Caspase-3 in Primary Striatal Stem Cells

Materials and Methods:

Striatal Stem Cell Production and Handling: On day E13 of gestation (E0 being the day a plug was observed) the pregnant female mouse was sacrificed in a manner which did not disturb her embryos (i.e. spinal dislocation). After sacrificing, the abdomen was sprayed with 70% ethanol. A 2 cm incision is made vertically up the abdomen. The horn was removed with sterile tweezers, and placed in PBS. The horn was cut open the horn to expose one embryo at a time and each embryo was placed in a separate 35 mm tissue culture dish containing 2 ml PBS. Each embryo was pinned to the dish and the cortex peeled off layer by layer to expose the striatum, which was then removed. The striatal tissue was placed in a 50 ml Falcon Tube containing cold Hank's Balanced Salt Solution (HBSS) on ice and the tissue allowed to settle at the bottom of the tube. The HBSS was replaced with 2 ml of Complete Neurocult Proliferation Media and the tissue triturated gently until only a few fragments of tissue remained. The supernatant was then collected and the fragments of tissue were triturated in 1 ml media. The supernatant was again removed and stored with the first supernatant that was collected.

At this point the number of cells can be counted using an aliquot of Trypan Blue (1/5 dilution). Cells were seeded at a density of 1 million cells per 25 ml of Complete Proliferation Media and grown in Complete Neurocult Proliferation Media for 4 days, at which time they were expanded if the plates appeared too confluent. If expansion was not necessary, the cells were allowed to grow for a few more days prior to harvesting. To expand the primary culture, half the cell suspension was placed in a new dish and 12.5 ml of Complete Proliferation Media was added to each of the initial plate and the expanded plate. After a few days, once most of the spheres had lifted off the dish, the sphere suspension was confluent. At this time, cells were either passaged in growth media, frozen, or plated onto a differentiation plate.

Thawing Striatal Stem Cells: Vials containing frozen cells were warmed in a 37° C. incubator until almost defrosted, then 1 ml of Complete Neurocult Proliferation Media was added drop-wise to each vial. The sphere suspension was transferred to a fresh tube containing 8 ml of Complete Neurocult Proliferation Media and the tube was centrifuged at 500 rpm for 5 min. at 4° C. Half the supernatant was removed by aspiration and the sphere pellet gently resuspended in five times using a 5 ml pipette. The sphere suspension was subsequently plated into 25 ml of Complete Neurocult Proliferation Media in a 10 cm dish and incubated at 37° C. Every day spheres were observed for viability and expansion. The culture was allowed to grow until many of the spheres had detached from the plate (5-10 days).

Plating and Differentiation of Striatal Stem Cells: In order to differentiate neurospheres, they must be plated onto a surface coated with Laminin and Poly-D-Lysine. The sphere suspension was collected from a 10 cm dish, placed in a 50 ml tube and centrifuged at 500 rpm for 5 min at 4° C. The supernatant was aspirated and discarded, then 2 ml of Complete Neurocult Proliferation Media was added and the sphere pellet gently triturated until the spheres were resuspended. The sphere suspension was then diluted in Complete Neurocult Proliferation Media to obtain the desired concentration (one 10 cm dish of neurospheres is appropriate for approximately 4 coated chamber slides). 2 ml aliquots of sphere suspension were plated per chamber and after a few hours at 37° C., the medium was changed to Complete Neurocult Differentiation media.

Paraformaldehyde (PFA) Fixing of Cells: Freshly made 4% PFA was used. Media was aspirated out of wells and each well was washed with 2 ml PBS for 3 min. After aspirating PBS out of the wells, 1.5 ml of 4% PFA was added to each well and incubated at room temperature for 10 min. (C2C12) or 30 min. (neurospheres). PFA was then aspirated out of the wells and each well was washed with 2 ml PBS for 3 min. After aspirating PBS out of the wells, 2 ml of PBS was added to each well and the plate stored at 4° C. until needed for staining.

Immunocytochemistry: Cells fixed with 4% PFA fixing and washed in PBS were incubated at RT in 1 ml 0.3% Triton X-100 (in PBS) for 10 min. and then washed three times with 2 ml PBS for 4 min. at RT. After aspiration of the PBS, the cells were blocked with 5% horse serum (in PBS) for 1 hr. at RT. After aspiration of the blocking solution, the cells were incubated at 4° C. overnight with 1 ml of primary antibody (in PBS+1% BSA for C2C12 cells and in PBS+10% HS for neurospheres) per well. (Primary Antibody dilutions used were: Active Caspase 3 (Rb Biovision) 1:50, Procaspase 3 (Ms Chemicon) 1:500, Mf20 (Ms) 1:50). Each well was then washed three times with 2 ml PBS for 5 min. at RT and the cells incubated at RT with 1 ml of secondary antibody (in PBS+1% BSA for C2C12 cells and in PBS+10% HS for neurospheres) per well for 1 hr. (cells were kept out of light from this point on). (Secondary Antibody dilutions used were: FITC (Rb Chemicon) 1:150, Rhodamine (Ms Chemicon) 1:150). Each well was washed three times with 2 ml PBS for 5 min. at RT and the cells were incubated at RT with 1 ml of DAPI (1:10000 dilution, in PBS) for 10 min. Each well was again washed with 2 ml PBS for 5 min. at RT (add PBS down side of well) and the cells suspended in 2 ml ddH₂O for mounting.

Results

Primary striatal neurospheres from E14 fetal mice can be maintained to proliferate in culture or induced to differentiate into different neural cell lineages. Differentiated neural tissue can form neurons or glia (including oligodendricytes and astrocytes as well as other lineages).

Neurospheres were cultured from E14 mice and incubated in the presence or absence of 1 μM of caspase-3 inhibitor z-DEVD-FMK (+/− caspase inhibitor). After incubation, neurospheres were scored for proliferative capacity and/or differentiation potential. Cells were fixed and immunostained for markers of neural lineages.

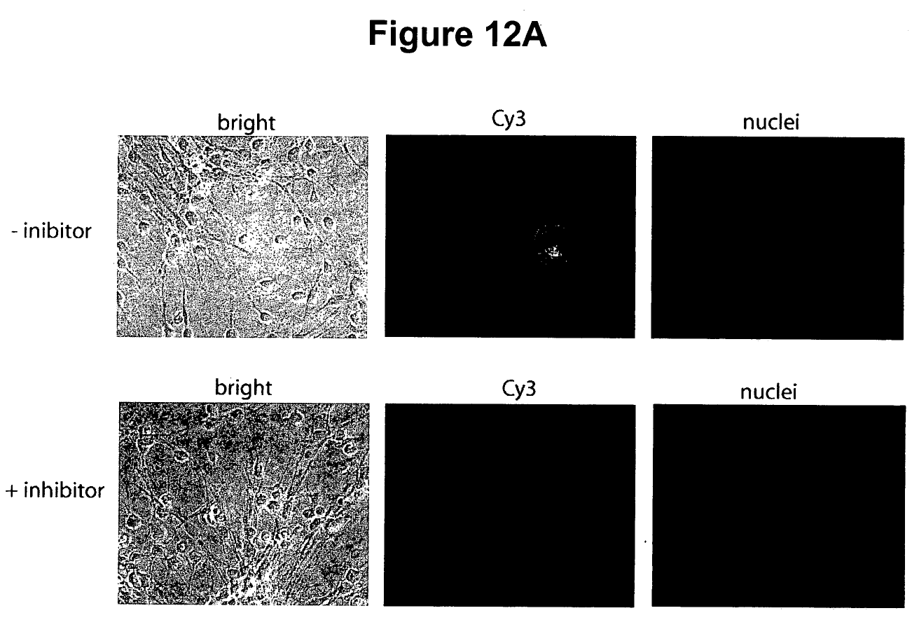

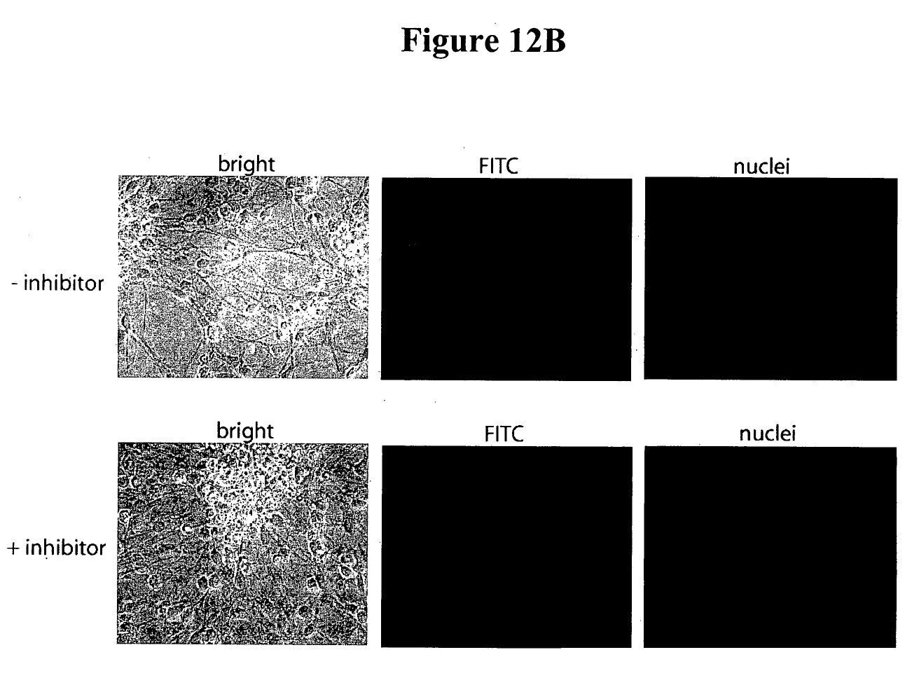

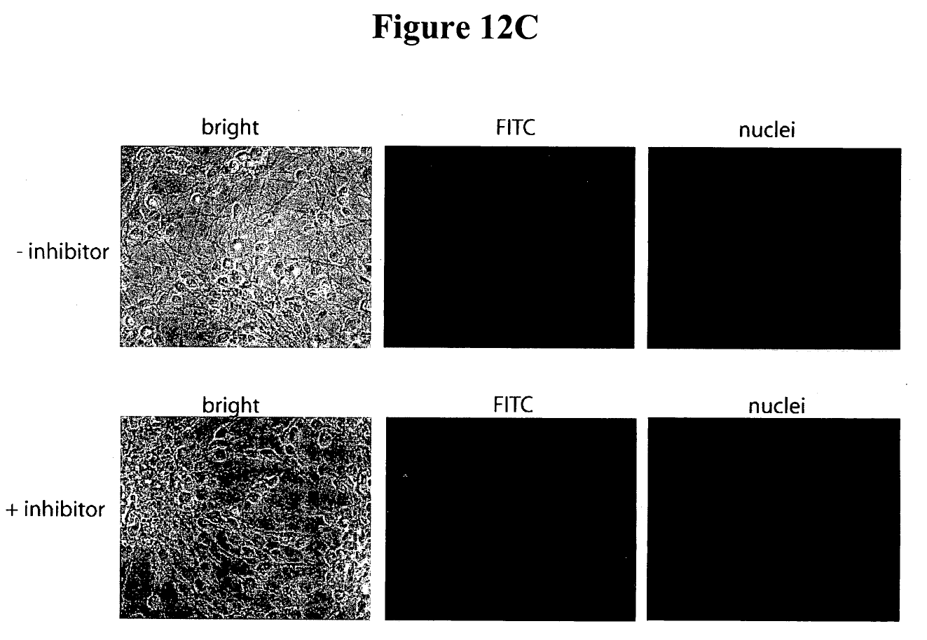

Proliferating striatal stem cells were stained with an anti-nestin antibody (marker for early neural cells) and visualized by counterstaining with a Cy3 fluorochrome. Cells were co-stained stained with DAPI to show nuclei. Typically, early neural progenitors are present during neurosphere growth. The results of this experiment, as shown in FIG. 12(A), indicate that caspase-3 inhibition using this particular inhibitor does not have an effect on cell cycling.

FIG. 12 (B) shows proliferating cells stained with an anti-myelin basic protein antibody (marker for oligodenricyte lineage) and visualized by counterstaining with a FITC fluorochrome. Cells were co-stained stained with DAPI to show nuclei. As expected, differentiated oligodendricytes are absent during neurosphere growth.

FIG. 12 (C) shows proliferating cells stained with an anti-glial fibrillary acid protein antibody (marker for astrocyte lineage) and visualized by counterstaining with a FITC fluorochrome. Cells were co-stained stained with DAPI to show nuclei. As expected, differentiated astrocytes are absent during neurosphere growth.

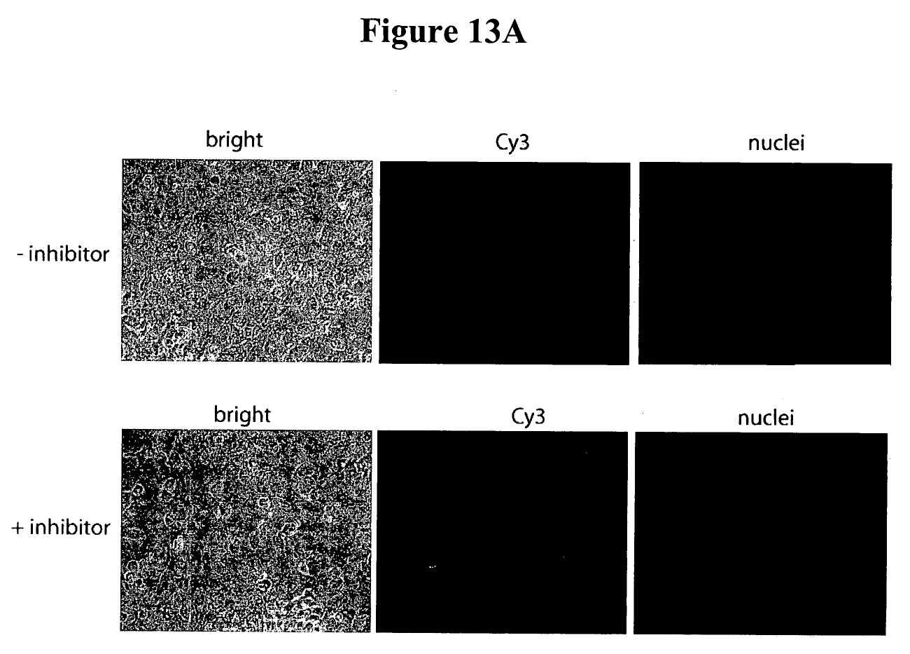

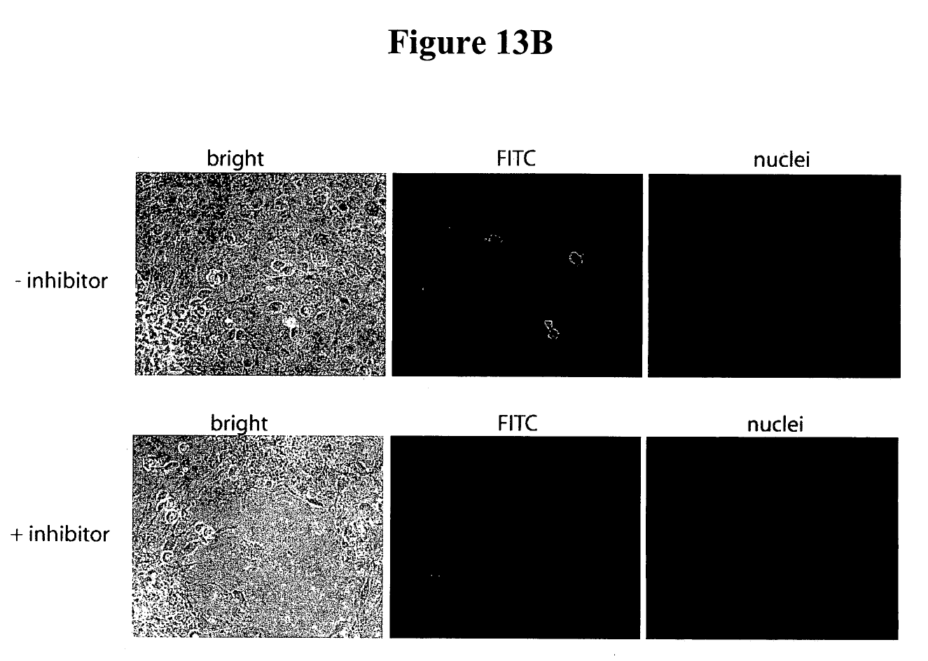

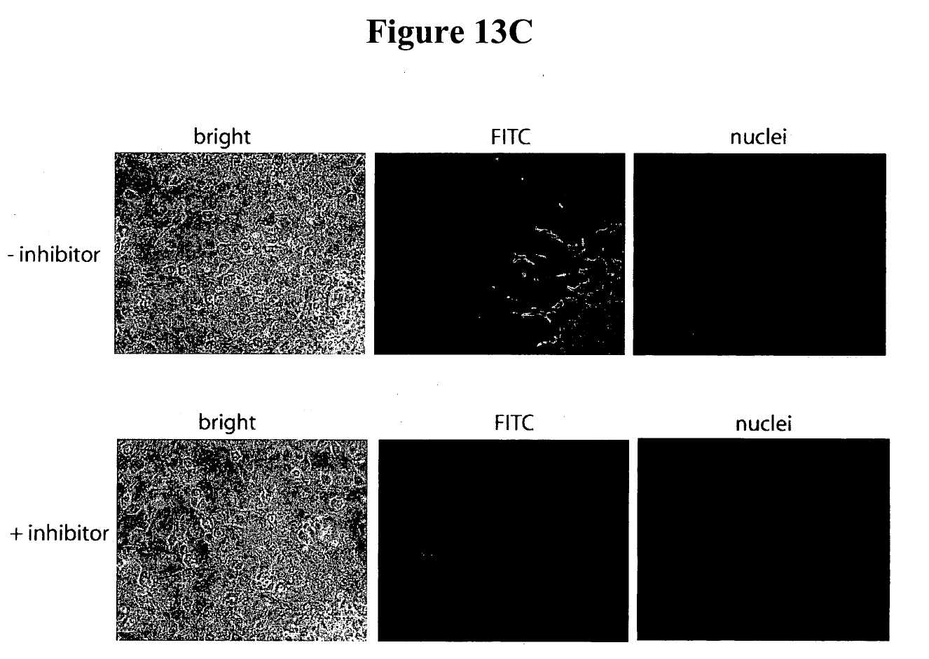

Differentiating striatal stem cells (48 h) were also stained with an anti-nestin antibody and visualized by counterstaining with a Cy3 fluorochrome. Cells were co-stained stained with DAPI to show nuclei. Early neural progenitors should be present during cell growth but not during cellular differentiation. FIG. 13 (A) shows that early neural progenitors are not present in cells that were not treated with caspase-3 inhibitor. However, in the presence of a caspase-3 inhibitor, cells retain their undifferentiated lineage as demonstrated by the presence of nestin. Thus, inhibition of caspase-3 inhibited neural cell differentiation.

FIG. 13 (B) shows differentiating cells that were stained with an anti-myelin basic protein antibody (marker for oligodendricytes) and visualized by counterstaining with a FITC fluorochrome. Cells were co-stained stained with DAPI to show nuclei. Differentiated neurospheres should show the presence of oligodendricytes. Cells incubated in the absence of caspase-3 inhibitor had differentiated properly as shown by the positive staining for oligodendricytes. Cells incubated in the presence of caspase-3 inhibitor did not stain positive for oligodendricytes indicating that differentiation of these cells has been delayed or halted. Thus, inhibition of caspase-3 inhibited oligodendricyte formation and therefore neural cell differentiation.

FIG. 13 (C) shows differentiating cells that were stained with a glial fibrillary acid protein antibody (marker for astrocytes) and visualized by counterstaining with a FITC fluorochrome. Cells were co-stained stained with DAPI to show nuclei. Astrocytes are part of the differentiated neuronal cell lineage. Cells incubated in the absence of caspase-3 inhibitor stained positive for an astrocytic marker, whereas cells differentiated in the absence of caspase-3 inhibitor showed no staining for astrocytes indicating that caspase-3 inhibition has also delayed or halted astrocyte formation. Thus, in the absence of caspase-3 activity, neuronal cell differentiation is inhibited.

### Example 4

### Cloning Human Pro-Caspase 3

In order to facilitate investigations into transient protein-protein interactions between pro-caspase 3 and other proteins in the cell, the pro-caspase 3 gene was PCR amplified to allow for cloning. Specifically, the human pro-caspase 3 gene (U26943) was PCR amplified from a cDNA pool generated by reverse-transcription PCR of HeLa cell RNA using primers that provided for insertion of EcoRI and PacI restriction sites at the 5′ and 3′ ends of pro-caspase 3 respectively: 5′ Primer: 5′-ATGACCATGATTACGAATTCATGGAGACAC-3′ (SEQ ID NO:5) 3′ Primer: 5′-CACTCTAGATTAATTAAAAAAATAGAGTTC-3′ (SEQ ID NO:6)

The PCR protocol was as follows: 95° C. 2 min, {95° C. 1 min, 55° C. 1 min, 72° C. 1 min} repeated for 30 cycles, 72° C. 10 min.

The PCR fragment can be subsequently digested with EcoRI and PacI and ligated into a suitable vector that has corresponding EcoRI and PacI sites. Following transformation of the recombinant vector into E. coli DH5alpha cells and selection of single bacterial colonies containing the recombinant vector, large scale preparation of plasmid DNA can be conducted. The plasmid DNA can be subsequently purified and sequenced for verification of the pro-caspase 3 insert.

### Example 5

### Method to Determine the Role of Caspase-3 in Early Differentiation of Various Stem Cells

Prior studies using a variety of tissue sources have determined that Hoechst dye stained cell suspensions reveal a Hoechst effluxing sub-population of cells (side population or SP cells). These cells possess stem cell-like activity, and are also characterized by sensitivity to the presence of verapamil, an inhibitor of multi-drug resistance-like proteins (Goodell, M. A., et al. (1996) J. Exp. Med. 183, 1797-1806; Gussoni, E., et al. (1999) Nature 401, 390-394; Jackson, K. A., et al. (1999) J. Clin. Invest. 107, 1395-1402.). A resident population of cardiac stem cells in the adult heart which can be manipulated experimentally has recently been reported (Hierlihy et al. (2002) FEBS Lett. 530, 239-243).

The role of caspase-3 in the early differentiation process of cardiac stem cells can be directly assessed using cardiac SP cells. Co-culture experiments can be performed using Z/AP derived cardiac SP cells and non-marked cardiomyocytes in the presence of caspase-3 and caspase-8 inhibitors. Immunohistochemical analyses can be performed to identify co-staining for β-galactosidase activity and for the expression of cardiomyocyte specific markers such as connexin-43, MEF2C and myosin heavy chain (using FITC-conjugated antibodies), following treatment with one or more modulator of caspase-3. The ability of caspase-3 null cardiac SP cells to participate in both in vitro and in vivo cardiomyocyte differentiation can then be assessed. A series of E1-deleted adenoviral vectors containing activated MST-1 (Ad-ActMST-1), a kinase dead MST-1 (Ad-kdMST-1) and wildtype MST-1 (Ad-wtMST-1) have been developed and can be used to infect the cardiac SP cells and test the response to co-culture induced differentiation. At this juncture it is important to note that the caspase-3 null mouse develops a functional myocardium. This apparent lack of phenotype may originate from compensatory activity of caspase-8, which has been shown to be capable of activating MST-1 (see Fernando et al., (2002) Proc. Natl. Acad. Sci. USA 99, 11025-11030), suggesting that either of caspase-3 or -8 may engage the differentiation process in the cardiac lineage. Co-culture experiments using caspase-3 null cardiac SP cells and primary cardiomyocytes, with and without caspase-8 inhibitors can be performed (immunohistochemistry is performed as above) to help identify the role(s) of each protease.

In addition, the ability of wildtype and caspase-3 null derived cardiac SP cells for cardiomyocyte reconstitution in vivo can be evaluated. To conduct this experiment, caspase null mice have been bred into the Z/AP marker strain enabling the location and fate of these cells to be tracked when injected into damaged myocardium.

These analyses may also be conducted using neural derived SP cells and embryonic day 14 derived cortical progenitor cells. The co-culture experiments use the Z/AP marker strain and appropriate neuronal markers.

Similarly, the effect of caspase-3 modulators on in vitro cultured SP cell populations derived from brain and bone marrow can be assessed. Cell counts, growth curves, markers of DNA replication and indications of differentiation, including documentation of differentiation specific gene products by Western blotting and RT-PCR can be conducted. These experiments can be followed by appropriate lineage-specific co-culture experiments to confirm that caspase-3 modulation does not alter the plasticity/viability of the cells.

In vivo experiments to complement those described above can also be performed. Varying concentrations of modulator can be used to treat mice (tail vein injection) followed by FACS analyses of retrieved single cell suspensions. In this manner the SP populations from various tissue sources (skeletal muscle, heart, brain and bone marrow) are measured and compared pre and post-caspase 3 modulation. The SP fractions from these same tissue sources in C57BL/6 wild-type, caspase-3 heterozygous and caspase-3 null mice (Frasch, S. C., et al., (1998) J. Biol. Chem. 273, 8389-8397; Kuida, K., et al., (1996) Nature 384, 368-372) (typically, n=5 for each genotype) as described (Cregan, S. P. et al., (1999) J. Neuro. 19, 7860-7869) can be compared.

In addition in vivo experiments in which an injury is separately induced in each target tissue (for example, coronary ligation to induce myocardial infarction, carotid artery occlusion to induce acute ischemic brain disease and direct skeletal muscle cardiotoxin injection to induce regenerative responses) can be performed. Varying concentrations of caspase-3 modulator(s) can be administered in order to expand and/or differentiate the desired stem cell population in vivo to effect an enhanced repair process.

### Example 6

### Neural Stem Cell Differentiation is Dependent Upon Caspase 3 Activity

Neurosphere culture: clonally-derived neurospheres were generated from the striatum of E14.5 CD1 murine embryos according to methods previously described (Reynolds, B. A., and Weiss, S. (1996) Dev Biol 175, 1-13). The neurospheres were maintained in NeuroCult Proliferation Medium (StemCell Technologies, Canada) supplemented with 20 ng/ml basic fibroblast growth factor (bFGF) as per manufacturer's instructions.

Neurospheres were sub-cultured no more than once per collection. The differentiation of neurospheres was initated by plating spheres on poly-L-ornithine coated plates in NeuroCult Differentiation Medium in the absence of bFGF, as per manufacturer's instructions. Samples were incubated with 2.0 μM of either caspase 3 inhibitor (z-DEVD.fmk) or caspase 8 inhibitor (z-IETD.fmk) (BioVison, USA) in growth medium for 24 h prior to plating. All cultures received fresh media and inhibitor every 24 h for the duration of the experiment. Neurospheres from caspase 3 null embryos (Fernando, P., et al., (2002) Proc Natl Acad Sci USA 99, 11025-11030) were cultured as described above.

Immunocytochemistry: Neurospheres were collected at selected time points, fixed in 4% paraformaldehyde and prepared for immunostaining as previously described (Fernando, P., et al., (2002) Proc Natl Acad Sci USA 99, 11025-11030)). The primary antibody concentrations for anti-nestin, -GFAP, -MBP and -active caspase 3 (BioVision, USA) were titrated on wildtype CD1 neurospheres prior to their experimental use. These antibodies were visualized by counterstaining with either FITC or Cy3 conjugated secondary antibodies (Chemicon USA). Nuclei were localized with 4′,6-Diamidino-2 phenylindole (DAPI) (Sigma, USA). Cells were visualized using either an Axiovert 100 for transmission microscopy or Axioplan 2 for fluorescent microscopy (Zeiss, Germany) at selected magnifications. Captured images were processed using AxioVision Viewer 3.0 software.

Immunoblot Assays: Immunoblot analyses were performed as previously described (Fernando, P., et al., (2002) Proc Natl Acad Sci USA 99, 11025-11030). All samples were processed at 4° C. to maintain protein integrity. To generate neurosphere cell extracts, cultured neurospheres were collected at the indicated time points and solubilized in modified RIPA buffer containing protease and phosphatase inhibitors. Cells were sheared 4-5 times through a 21 gauge needle and mixed by 360° rotation at 4° C. for 30 minutes. Nestin 3 (AbCam, UK) was detected following the separation of total cellular proteins through a 6% acrylamide gel and Western transfer. Antibodies against neuronal class III α-tubulin and GFAP (AbCam, USA), active caspase 3 (BioVision, USA), 2′3′ cyclic nucleotide 3′ phosphohydrolase (CNPase) (Chemicon, USA) and p38α (Santa Cruz, USA) were used to detect proteins following separation by 10% polyacrylamide electrophoresis and Western transfer. In some instances, sample aliquots were sonicated following cell lysis in order to detect total cellular poly-ADP ribose polymerase (PARP) (BioVison, USA). Soluble PARP was assayed from unsonicated samples. Protein bands from Western blot assays were quantitated using Scion Image (Scion Corporation, USA).

Immunoprecipitation/Kinase activity assays: Immunoprecipitation/kinase analyses were performed as previously described (Fernando, P., et al., (2002) Proc Natl Acad Sci USA 99, 11025-11030). Cellular lysates from neurospheres were generated as described above. Antibodies against apoptotic signal-regulating kinase 1 (ASK1) (Upstate, USA), p21 activated kinase 1 (PAK1) (Cell Signaling Technologies, USA), and p38 MAPK (Santa Cruz, USA) were used to immunoprecipitate respective proteins. Samples were mixed with Protein G-sepharose beads for 1 h, washed and then mixed with kinase assay buffer containing myelin basic protein (MBP) and γ-32P[ATP]. Samples were reacted at 30° C. for 30 min and separated using SDS-PAGE. In some instances, samples were transferred to PVDF membrane following electrophoresis. To quantify kinase activities, MBP acrylamide bands were excised and solubilized in scintillation fluid prior to 32P-liquid scintillation analyses. In all cases, an activated recombinant mitogen activated protein kinase activated protein-2 (MAPKAP kinase-2) (Upstate, USA) was used as an internal control for liquid scintillation analyses.

Flow Cytometry: Annexin-V-propidium iodide (PI) analysis of apoptotic and nectrotic cells was performed using an Annexin-V-FLUOS staining kit (Roche, USA). Analyses were performed using a Beckman-Coulter ALTRA flow cytometer. To detect Annexin-V and PI positive cells, flow cytometric channels were gated using cells without stain, stained with PI alone and stained with Annexin-V alone. Cells that had dissociated from their neurosphere bodies were gently collected without excessive disruption of the sphere/cell body. These cells were chosen for flow cytometry analyses. Each sample was gated to control for cellular debris.

Quantitation of Neurite Extensions: Neurites from differentiating neurospheres were estimated from a total of 6-8 neurospheres per condition over three independent experiments. To quantitated neuritis, the average diameter of an individual neurosphere was estimated from the vertical and horizontal distance. The number of extensions leaving the neurosphere body were then counted and an ratio of neurosphere extensions:average neurosphere diameter was calculated for each. All measurements including neurosphere diameter were evaluated using Scion Image.

Results

Endogenous Caspase 3 Activity is Elevated During Neurosphere Differentiation

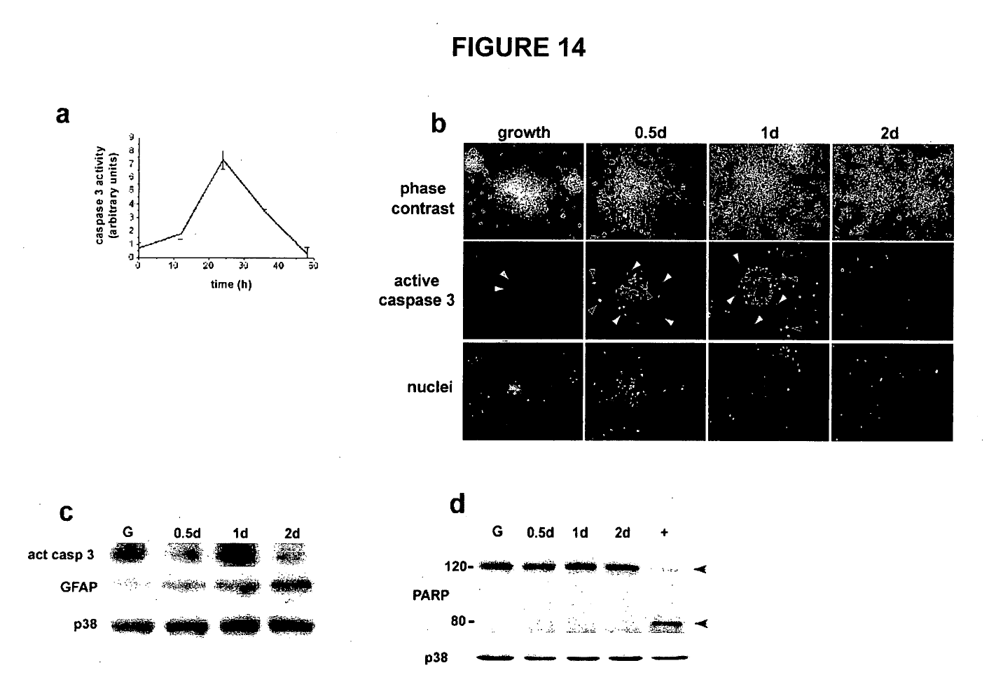

As a step toward understanding the role of caspase 3 during neural differentiation, we utilized primary derived neuronal stem cells as a model system. Primary striatal neurospheres were obtained from CD1 murine embryos at 14.5dpc and maintained and expanded as described above. During neurosphere differentiation, a substantial increase in caspase 3 activity was measured (FIG. 14a). The activity peaked by approximately 24 h and then decreased thereafter. A similar pattern of caspase 3 activity was detected in differentiating myoblasts (Fernando, P., et al., (2002) Proc Natl Acad Sci USA 99, 11025-11030).

Under normal growth conditions, a measurable level of caspase 3 activity was observed in single cells surrounding the neurosphere (FIG. 14b). Immunoblot analyses from cultured neurosphere lysates confirmed these observations (FIG. 14c). After 12 h of differentiation, caspase 3 activity was primarily localized to the peripheral regions of the neurosphere and within the surrounding cell population. As observed with fluorometric assays, caspase 3 activity remained elevated at 24 h and then declined by 48 h. Neurosphere differentiation was confirmed by the increase in the astrocytic marker GFAP, following 48 h of low serum/bFGF withdrawal (differentiation media). In addition, caspase 3 activation did not result in the cleavage of poly(ADP) ribose polymerase (PARP), an end-product of apoptosis (FIG. 14d). Interestingly, the variable pattern of caspase 3 activity was similar to that observed previously in differentiating skeletal myoblasts (Fernando, P., et al., (2002) Proc Natl Acad Sci USA 99, 11025-11030). Moreover, the absence of PARP cleavage products suggested the potential for a non-death role of caspase 3 during neurogenesis.

Caspase 3 Inhibition Does Not Affect Apoptosis in Differentiating Neurosphere Cultures

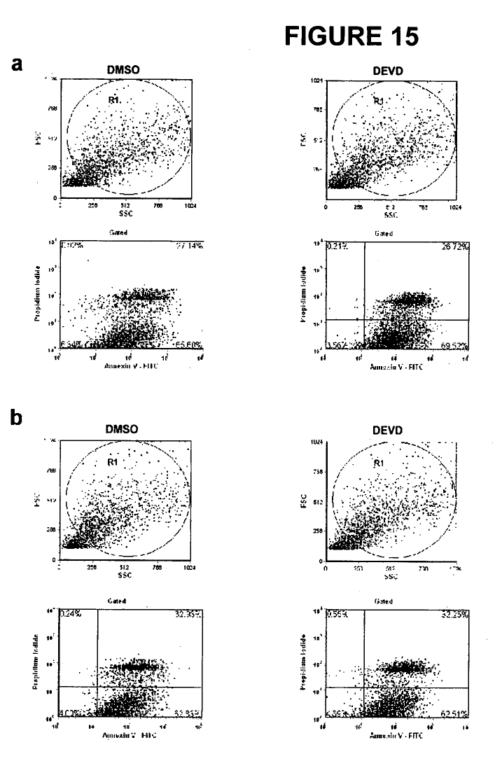

As in most developmental processes, a population of cells is required to undergo apoptosis prior to the differentiation of a separate pool of cells. Clearly, the inhibition of caspase 3 may adversely affect this process. To address this issue, we measured the degree of apoptosis that occurred during differentiation in caspase 3 inhibited and uninhibited cells using the specific caspase 3 peptide inhibitor zDEVD.fmk and Annexin V-propidium iodide (PI) staining coupled to cytometric analyses (FIG. 15). Annexin V detects cells that are either apoptotic or necrotic whereas propidium iodide is able to detect necrotic cells. Together, Annexin-V-PI analyses identify the population of cells that are apoptotic, i.e. Annexin-V positive/PI negative. To control for the heterogeneity of cells within neurospheres as described previously (Bez et al., (2003) Brain Res 993, 18-29), we performed our analyses on cells that had dissociated from the neurosphere. After 12 h of treatment in differentiation media, approximately 27% of the cell population from both control and caspase 3 inhibited cells were Annexin-VI positive/PI positive (FIG. 15a). Interestingly, a small increase was observed after 24 h but the level of apoptosis was similar in both treatments (33%, FIG. 15b). Our results clearly indicate that caspase 3 inhibition did not affect apoptosis in caspase 3 inhibited cells relative to uninhibited cells during neurosphere differentiation.

Inhibition of Caspase 3 Activity Leads to a Reduction in Neurogenic Proteins

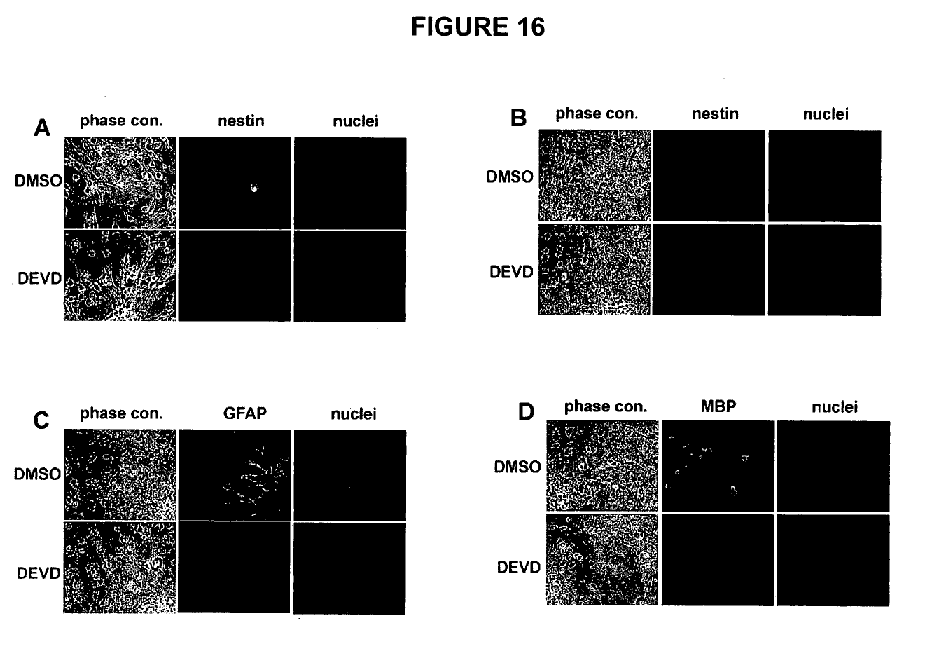

To directly assess the function of caspase 3 that is normally associated with neural growth and differentiation, neurospheres were cultured with the caspase 3 peptide inhibitor z-DEVD.fmk, and measured for their ability to engage the differentiation program. Under growth conditions, neurospheres readily stained for nestin, an intermediate protein highly expressed in early neuronal progenitors (Lendahl et al., Cell 60, 585-595). Peptide inhibition of caspase 3 activity (DEVD panels) did not affect nestin expression (FIG. 16a). Subsequently, we compared neural differentiation without caspase 3 activity. Untreated CD1 neurospheres (DMSO panels) displayed a rapid decline in nestin expression upon differentiation (FIG. 16b) with concurrent increases in glial fibrillary acidic protein (GFAP) (FIG. 16c) and myelin basic protein (MBP) (FIG. 16d). Increased expression of GFAP, MBP are characteristic with the early formation of astrocytes and oligodendrocytes respectively (Gage, F. H. (2000) Science 287, 1433-1438). Interestingly, inhibition of caspase 3 activity resulted in the sustained expression of nestin under differentiation conditions. Furthermore, we observed a significant decrease in GFAP and MBP expression in caspase 3 inhibited neurospheres (FIG. 16b-d, DEVD panels). In other experiments, the caspase 3 inhibitor Ac-DEVD-CHO was used however this inhibitor often resulted in high levels of cytotoxicity.

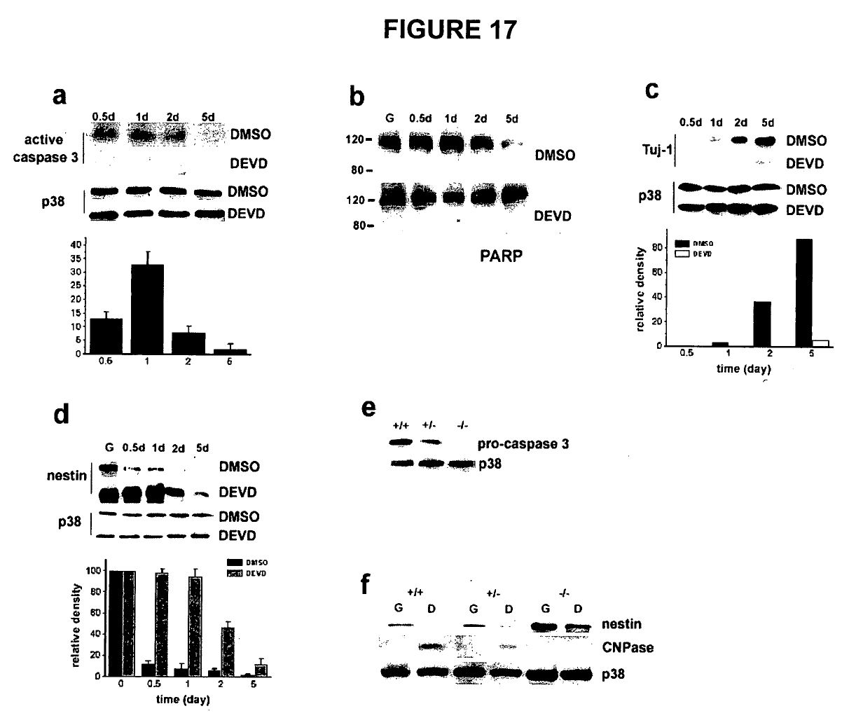

We confirmed our immunocytochemical results with Western blot experiments. As previously stated in differentiating neurospheres, caspase 3 activity peaked by 24 h and decreased thereafter (FIG. 17a). Quantitation of these results confirmed our initial observations shown in FIG. 14. Importantly, caspase 3 activity was effectively inhibited in DEVD treated neurospheres throughout the differentiation time course. PARP cleavage was not apparent in differentiating neurospheres as we did not observe the 85 kDa cleaved PARP fragment (FIG. 17b). Although PARP cleavage is typically associated with apoptosis, the dramatic decrease in soluble PARP protein after 120 h of differentiation may suggest a regulatory role for PARP in differentiating neurospheres. Indeed, PARP was recently described to behave as a histone modifier/DNA transcriptional activator (Kraus et al., (2003) Cell 113, 677-683). Our results indicated that caspase 3 activity is responsible for the alterations in gene expression normally associated with neurosphere differentiation.

The neuronal marker β-III tubulin accumulated within 2 d under normal differentiation conditions and continued to increase following further incubation (FIG. 17c, DMSO panel). In contrast, β-III tubulin did not increase when caspase 3 activity was inhibited (FIG. 17c, DEVD panel) indicating the absence of a neuronal phenotype, although a very low level of β-III tubulin was detectable in these cells after 5 d. The persistence of nestin in z-DEVD treated neurospheres was confirmed by immunoblot analysis (FIG. 17d). As expected, nestin levels remained elevated during the early periods of differentiation. However, a decrease in nestin was observed after an extended period in differentiation media with z-DEVD treatment (4 d DEVD panel). Taken together, these results suggest that neurosphere differentiation is delayed during caspase 3 inhibition.

To validate our observations from the caspase 3 peptide inhibition model, we examined the effect on neurogenesis in a caspase 3 null murine model (FIG. 17e). Caspase 3 heterozygote breeding gives rise to few caspase 3 null embryos which exhibit perinatal lethality (Cregan et al., (1999) J Neurosci 19, 7860-7869; Kuida et al., (1996) Nature 384, 368-372). The neurospheres generated from these mice are not robust and are consequently difficult to maintain ex vivo. Typically, we were unable to culture these cells beyond the first passage since many of the dissociated cells following the subculture routine failed to aggregate and form neurosphere bodies. Neurospheres generated from wildtype (+/+) and caspase 3 heterozygous (+/−) embryos showed drastic reductions in nestin expression following 2 d in differentiation media. In contrast, nestin remained elevated in neurospheres from caspase 3 mutant (−/−) embryos (FIG. 17f). To expand our observations on neurogenic specific proteins, we measured the levels of 2′3′ cyclic nucleotide 3′phosphohydrolase (CNPase), an enzyme found in oligodendrocytes. CNPase was readily detected in caspase 3 wildtype neurospheres. A smaller amount of CNPase was found in heterozygote neurospheres however CNPase was not detected in the caspase 3 mutant cultures (FIG. 17f).

Reduced Neurite Extensions in Caspase 3 Inhibited Neurospheres

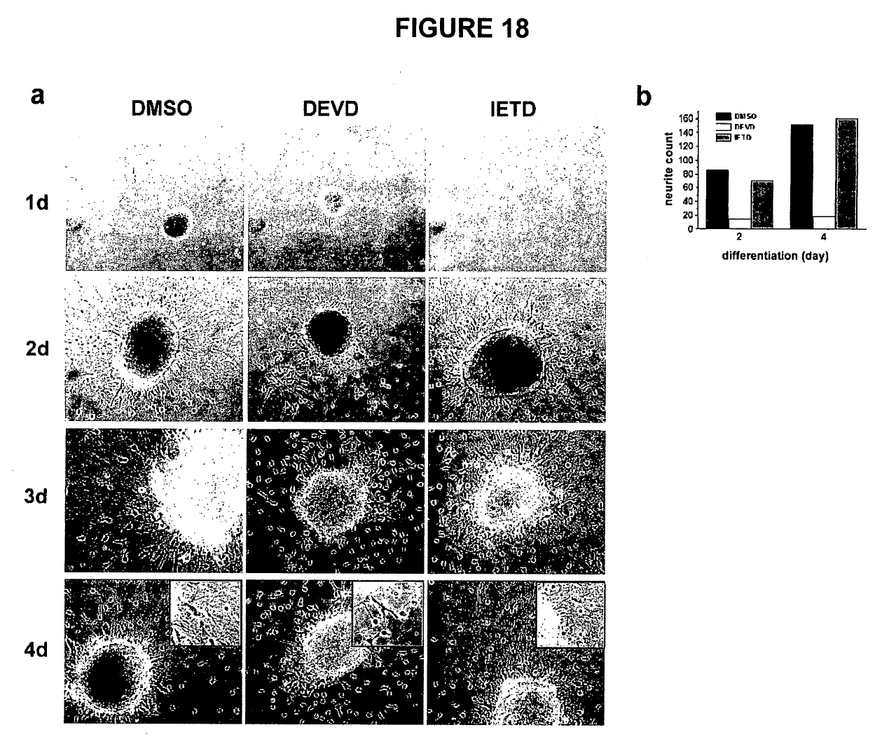

We next examined whether caspase 3 activity influenced the morphologic features that typically segregate with the differentiation of neurosphere cultures. The induction of differentiation resulted in an increasing level of neurite extensions with extensive networks extending from the neurosphere bodies (FIG. 18, DMSO panels and DMSO 4 d inset). These observations parallel the dendritic and axonal branching that occurs in vivo. However, peptide inhibition of caspase 3 activity led to a dramatic reduction in the formation of these projections (FIG. 18, DEVD panels and DEVD 5 d inset). Interestingly, peptide inhibition of caspase 8 (z-IETD.fmk) did not affect morphological changes in neurosphere differentiation (compare FIG. 18, 5 d panel inset), although pan-inhibition of caspase activity (z-VAD.fmk) gave results similar to caspase 3 inhibition (not shown). Therefore, our results suggest that morphologic adaptations that accompany neurogenesis are dependent on a caspase 3 specific activity.

Protein Kinase Activity is Reduced in Caspase 3 Inhibited Neurospheres.

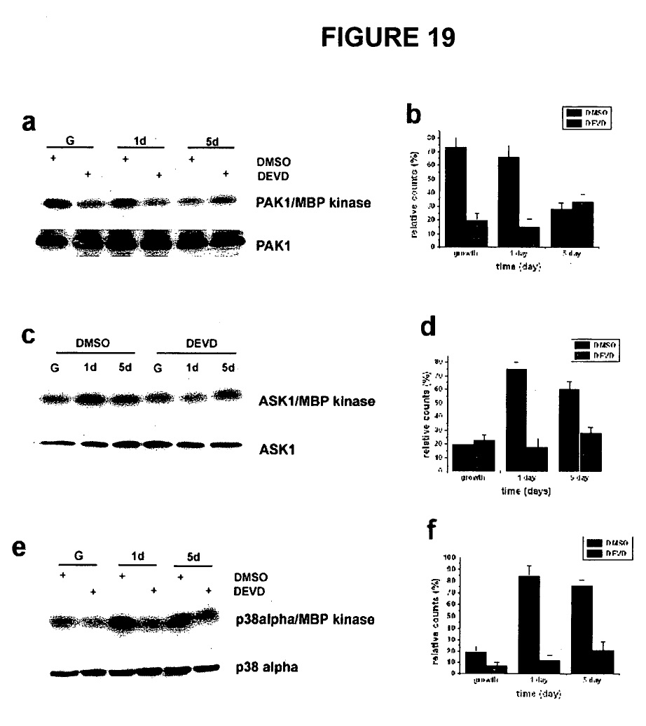

Caspase 3, when processed to its active form, is capable of targeting and activating many protein kinases that mediate cellular differentiation. The p21 activated kinase gamma (PAK2) is known to be activated in response to multiple stimuli leading to the induction of apoptosis (Bokoch, G. M. (1998) Cell Death Differ 5, 637-645). Under our experimental conditions, we did not observe differences in PAK2 activity between caspase 3 inhibited and control treated cultures during neurosphere differentiation (data not shown). Interestingly, PAK1 activity was reduced in DEVD treated neurospheres (FIG. 19a,b), although PAK1 has not been demonstrated as a caspase 3 substrate. In addition to PAK1, ASK1 kinase activity was also noted to decrease in DEVD treated cells.

ASK1 activity was reduced when cells were treated with differentiation media containing z-DEVD (DEVD condition) compared to differentiation media alone (DMSO condition) (FIG. 19c,d). Both PAK1 and ASK1 have been linked to the downstream activity of p38 MAPK. We observed very low levels of p38α kinase activity in neurospheres incubated with differentiation media under caspase 3 inhibition. (FIG. 19e,f). These results suggest that protein kinase activity normally associated with neurogenesis is attenuated when caspase 3 activity is inhibited.

Activation of caspase proteases are accepted as the penultimate step in a hierarchical death pathway. Despite this widely held belief, our data indicates that caspase 3 activity is also required for a vital non-death function in the neuronal lineage. This study, together with previous observations from our laboratory suggests that activation of caspase 3 represents a common point of origin for induction of cell differentiation.

Clearly, increased caspase 3 activity leads to the activation of cellular remodeling kinases such as PAK1 and ASK1. In turn, these kinases also activate downstream protein kinases such as p38 and are known to target the nucleus and subsequently phosphorylate differentiation specific transcription factors. Inhibition of caspase 3 activity in our study resulted in attenuated levels of p38α, a kinase that phosphorylates the myocyte enhancer factor 2 (MEF2) family. MEF2 is highly expressed in neural cell populations and its activity is required for neurogenesis (Okamoto et al., (2000) Proc Natl Acad Sci USA 97, 7561-7566; Lyons et al., (1995) J Neurosci 15, 5727-5738; Skerjanc, 1. S., and Wilton, S. (2000) FEBS Lett 472, 53-56).

Given the observations reported here, the targeted inhibition of caspase 3 activity may be problematic as a therapeutic intervention for treating neural related illnesses. For example, animal studies using ischemic brain injury models have demonstrated that neural precursor cells/stem cells migrate to the areas of focal damage and assist in tissue remodeling and neuronal repopulation (Li et al., (2000) Stroke 31, 176-182). Clearly, for neural stem cells to augment the repair process, the differentiation program must proceed unhindered. Our data suggests that inhibition of caspase 3 activity may limit such a repair process by disrupting the normal pattern of neurogenesis. Further our data suggests that activation of caspase 3 is required for stem cell differentiation.

While the foregoing invention has been described in some detail for purposes of clarity and understanding, it will be appreciated by one skilled in the art from a reading of this disclosure that various changes in form and detail can be made without departing form the true scope of the invention and appended claims.

All citations are herein incorporated by reference.

The embodiments of the invention in which an exclusive property or privilege is claimed are defined as follows:

1. A method of modulating stem cell differentiation comprising contacting a stem cell, or a population of stem cells, with one or more modulators of caspase-3 activity.

2. The method according to claim 1 wherein said one or more modulators are selected from the group of pro-caspase 3, active-caspase 3, Mammalian Sterile Twenty-like kinase 1 (MST1), MEKK1, ASK1, SLK, MKK6, MKK3, p38α, p38γ, XIAP, c-IAP2, c-IAP1, survivin, caspase-1, caspase-8, caspase-9, caspase-10, granzyme B, I-FLICE and CrmA, or a combination thereof.

3. The method according to claim 1, wherein said one or more modulators increase the activity of caspase-3 and induce stem cell differentiation.

4. The method according to claim 1, wherein said one or more modulators attenuate the activity of caspase-3 and inhibit stem cell differentiation.

5. The method according to claim 1, wherein said stem cell, or population of stem cells comprise muscle stem cells, cardiac stem cells, neural stem cells, cortical stem cells, bone marrow stem cells or a combination thereof.

6. The method according to claim 1, wherein said stem cell, or population of stem cells, are in vivo.

7. The method according to claim 1, wherein said stem cell, or population of stem cells, are ex vivo.

8. The method according to claim 1, wherein said stem cell, or population of stem cells, are in vitro.

9. The method according to claim 1, wherein said stem cell, or population of stem cells, are contacted sequentially with a modulator that attenuates the activity of caspase-3 and inhibits stem cell differentiation and a modulator that increases the activity of caspase-3 and induces stem cell differentiation.

10. The method of claim 5, wherein said stem cells comprise neuronal stem cells.

11. The method of claim 10, wherein said neuronal stem cells are neuroblasts.
